(19) United States
(12) Patent Application Publication
(10) Pub. No.: US 2019/0330336 A1
(43) Pub. Date: Oct. 31, 2019

(54) ANTI-LAG3 ANTIBODIES, COMPOSITIONS COMPRISING ANTI-LAG3 ANTIBODIES AND METHODS OF MAKING AND USING ANTI-LAG3 ANTIBODIES

(71) Applicant: SUTRO BIOPHARMA, INC., SOuth San Francisco, CA (US)

(72) Inventors: Ryan STAFFORD, Emeryville, CA (US); Alice YAM, Tiburon, CA (US); John LEE, San Francisco, CA (US); Aaron SATO, Burlingame, CA (US)

(21) Appl. No.: 15/777,048

(22) PCT Filed: Nov. 18, 2016
(86) PCT No.: PCT/US2016/062943

§ 371 Date: May 17, 2018

Related U.S. Application Data
(60) Provisional application No. 62/257,653, filed on Nov. 19, 2015.

(51) Int. Cl. C07K 16/28

(52) U.S. Cl. CPC C07K 16/2803; C07K 2317/565; C07K 2317/76; C07K 2317/52; C07K 2317/55; C07K 2317/24; C07K 2317/40; C07K 2317/622; C07K 2317/92; C07K 2317/21

(57) ABSTRACT

Provided herein are antibodies that selectively bind to LAG3 and its isoforms and homologs, and compositions comprising the antibodies. Also provided are methods of using the antibodies, such as therapeutic and diagnostic methods.

### FIELD

Provided herein are antibodies with binding specificity for lymphocyte-activation gene 3 (LAG3) and compositions comprising the antibodies, including pharmaceutical compositions, diagnostic compositions, and kits. Also provided are methods of making anti-LAG3 antibodies, and methods of using anti-LAG3 antibodies, for example, for therapeutic, diagnostic purposes, and research purposes.

### BACKGROUND

The lymphocyte activation gene 3 (LAG3) was discovered in 1990. Triebel et al., 1990, J. Exp. Med. 171:1393-4053. It was identified as selectively transcribed in activated natural killer (NK) cells and T lymphocytes. See id. The LAG3 protein was originally described as a type I membrane protein of 498 amino acids including a signal peptide, an extracellular region, a transmembrane region, and a cytoplasmic region. See id. The extracellular region has four Ig domains, and the whole protein has sequence similarity to CD4. See id.

LAG3 is selectively expressed in regulatory T cells, and its natural ligand is MHC class II. Huang et al., 2004, Immunity 21:503-513. Regulatory T cells are important for maintaining immune tolerance to limit autoimmunity and in regulating lymphocyte expansion. See id. They also suppress natural immune responses to parasites and viruses, and they have suppressed antitumor immunity induced by therapeutic vaccines. See id. Antibodies to LAG3 were shown to inhibit suppression by induced regulatory T cells. See id. Antibody targeting of LAG3 has been shown to enhance antitumor immunity in animal models of cancer. Pardoll, 2012, Nature Rev. Cancer 12:252-264; Jing et al., 2015, 1 Immunother. Cancer 3:2-29. LAG3 is an immune checkpoint protein target for active drug development, and clinical trials have been proposed for antibodies to LAG3 for the treatment of solid tumors.

In view of the role of LAG3 in multiple disease processes, there is a need for improved methods of modulating the immune regulation of LAG3 and the downstream signaling processes activated by LAG3. Moreover, given the role of LAG3 in several diseases, there is also a need for therapeutics that specifically target cells and tissues that express LAG3.

### SUMMARY

Provided herein are antibodies that specifically bind to LAG3. In some embodiments, the antibodies bind human LAG3. In some embodiments, the antibodies also bind homologs of human LAG3. In some aspects, the homolog is a cynomolgus monkey homolog.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, or a variant thereof. In some aspects, the variant is a variant with one or more conservative amino acid substitutions.

Also provided are compositions comprising the antibodies. In some embodiments, the composition is a pharmaceutical composition. In some embodiments, the pharmaceutical composition is for the treatment or diagnosis of a disease or condition, as described further elsewhere in this disclosure. In some embodiments, the pharmaceutical composition is a composition for parenteral administration.

This disclosure also provides methods of making the anti-LAG3 antibodies provided herein. The antibodies can be made, for example, in any suitable cell or organism. The antibodies can also be made in a cell-free reaction mixture.

Also provided are methods of using the anti-LAG3 antibodies provided herein. In some embodiments, the method of use is a method of treatment. In some embodiments, the method of use is a diagnostic method. In some embodiments, the method of use is an analytical method. In some embodiments, the method of use is a method of purifying and/or quantifying LAG3.

In some embodiments, the antibodies are used to treat a disease or condition. In some aspects, the disease or condition is a cancer.

### BRIEF DESCRIPTION OF THE DRAWINGS

FIG. 1 provides an alignment of the V_{H} sequences provided herein. CDRs according to Chothia are outlined, and CDRs according to Kabat are underlined.

FIG. 2 provides an alignment of the V_{L} sequences provided herein. CDRs according to Chothia are outlined, and CDRs according to Kabat are underlined.

### DETAILED DESCRIPTION

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “combinations thereof” includes every possible combination of elements to which the term refers to. For example, a sentence stating that “if α₂ is A, then α₃ is not D; α₅ is not S; or α₆ is not S; or combinations thereof” includes the following combinations when α₂ is A: (1) α₃ is not D; (2) α₅ is not S; (3) α₆ is not S; (4) α₃ is not D; α₅ is not S; and α₆ is not S; (5) α₃ is not D and α₅ is not S; (6) α₃ is not D and α₆ is not S; and (7) α₅ is not S and α₆ is not S.

The terms “LAG3” and “LAG3 antigen” are used interchangeably herein. LAG3 is also known by a variety of synonyms, including lymphocyte-activation gene 3, CD223, cluster of differenetiation 223, and FDC, among others. Unless specified otherwise, the terms include any variants, isoforms and species homologs of human LAG3 that are naturally expressed by cells, or that are expressed by cells transfected with an LAG3 gene. LAG3 proteins include, for example, human LAG3 (GI: 15928632; SEQ ID NO:1). In some embodiments, LAG3 proteins include cynomolgus monkey LAG3 (GI: 544483249; SEQ ID NO:2). In some embodiments, LAG3 proteins include murine LAG3 (GI: 112293275; SEQ ID NO:3). However, as discussed in detail elsewhere in this disclosure, in some embodiments the antibodies provided herein do not bind murine LAG3 proteins. The antibodies provided herein bind to an extracellular domain of LAG3.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. An “LAG3 antibody,” “anti-LAG3 antibody,” “LAG3 Ab,” “LAG3-specific antibody” or “anti-LAG3 Ab” is an antibody, as described herein, which binds specifically to the antigen LAG3. In some embodiments, the antibody binds the extracellular domain of LAG3.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at http://www.bioinf org.uk/abs/abnum/, and described in Abhinandan and Martin, Immunology, 2008, 45:3832-3839, incorporated by reference in its entirety.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

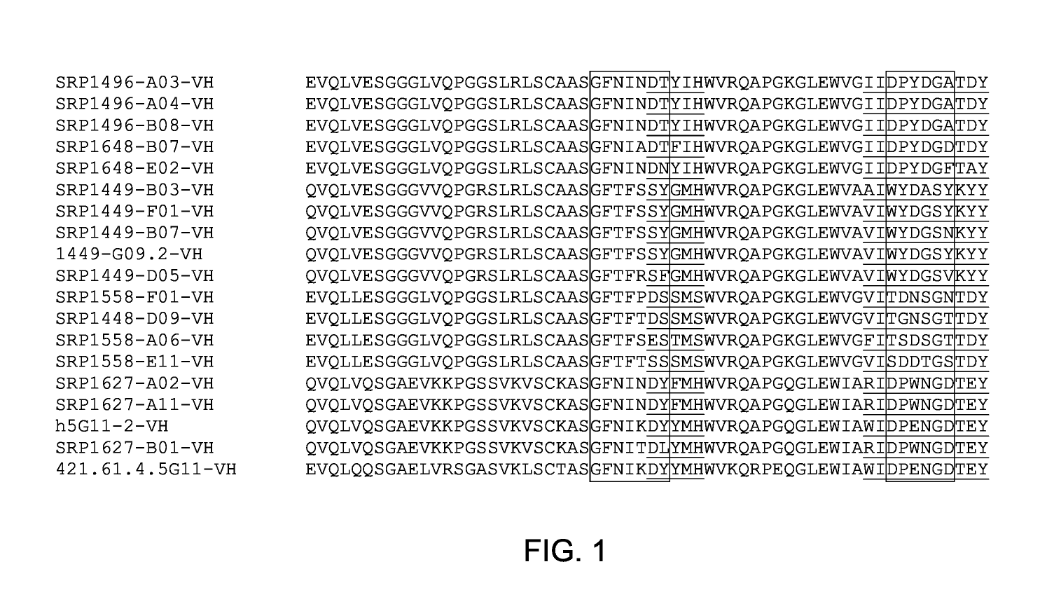

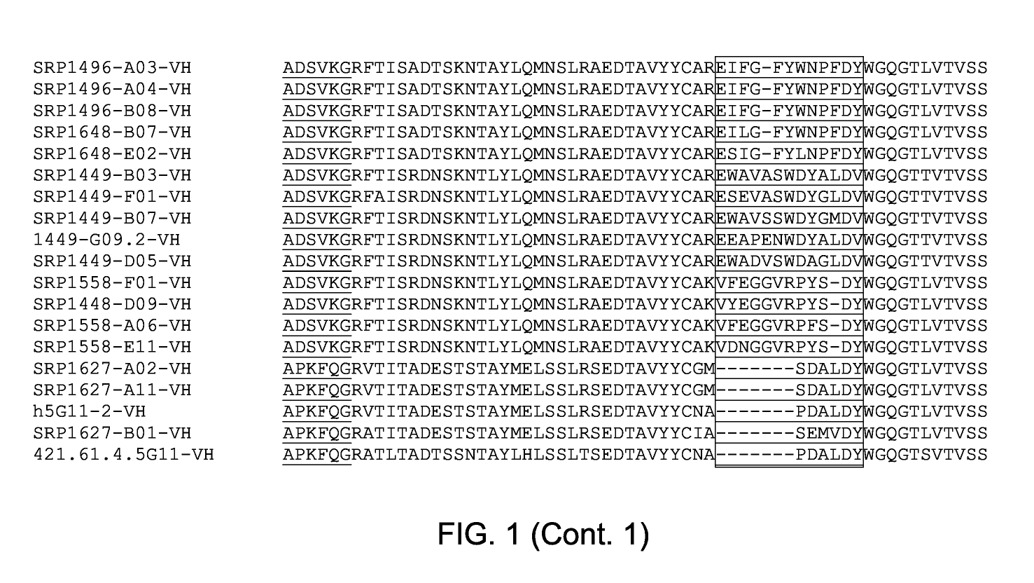

*The C-terminus of CDR-H1, when numbered using the Kabat numbering convention, varies between H32 and H34, depending on the length of the CDR, as illustrated in FIG. 1.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H}O of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with B-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO:188 or 189. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO:180, or a portion thereof, or SEQ ID NO:185. SEQ ID NO:180 provides the sequence of C_{H1}, C_{H2}, and C_{H3} of the human IgG1 constant region. SEQ ID NO:185 provides the sequence of the constant region used in the illustrative scFv-Fc antibodies provided herein.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least about 80%, 85%, 90%, 95%, or 99% by weight. In some embodiments, an isolated antibody is purified to at least about 80%, 85%, 90%, 95%, or 99% by volume. In some embodiments, an isolated antibody is provided as a solution comprising at least about 85%, 90%, 95%, 98%, 99% to 100% by weight. In some embodiments, an isolated antibody is provided as a solution comprising at least about 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at about 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., LAG3). In one exemplary assay, LAG3 is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least about 50%, at least about 60%, at least about 70%, at least about 80%, or at least about 90%.

The term “epitope” means a portion of an antigen capable of specific binding to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to LAG3 variants with different point-mutations, or to chimeric LAG3 variants as described further in the Examples provided herein.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions for
one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a cancer that can be treated or diagnosed with an antibody provided herein. In some embodiments, the cancer is a cancer of epithelial origin.

### 2. Antibodies

Provided herein are antibodies that selectively bind human LAG3. In some aspects, the antibody selectively binds to the extracellular domain of human LAG3.

In some embodiments, the antibody binds to a homolog of human LAG3. In some aspects, the antibody binds to a homolog of human LAG3 from a species selected from monkeys, mice, dogs, cats, rats, cows, horses, goats and sheep. In some aspects, the homolog is a cynomolgus monkey homolog.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 6, 7, 8, 9, 10, 11, 12, or 13 residues in length.

In some aspects, the Kabat/Chothia CDR-L1 of the antibody is 11, 12, 13, 14, 15, 16, 17, or 18 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the scFv-Fc fragment comprises a constant region wherein the constant region comprises SEQ ID NO:185. The constant region in SEQ ID NO:185 differs from the human IgG1 constant region of SEQ ID NO:180 in several respects. First, the sequence in SEQ ID NO:185 comprises the linker AAGSDQ (SEQ ID NO:99). SEQ ID NO:185 also does not comprise the CH1 domain of the IgG1 constant region. SEQ ID NO:185 further comprises a C220S (EU numbering system) mutation, which removes an unpaired cysteine reside that is not needed when the light chain constant region is not present (e.g., in an scFv-Fc format). SEQ ID NO:185 further comprises two, optional, P to S mutations (P230S and P238S by the EU numbering system). Either or both of these serine residues can be reverted to the naturally occurring proline residues. Finally, SEQ ID NO:185 comprises an aspartic acid (D) residue at EU position 356 and a leucine (L) residue at EU position 358. In contrast, SEQ ID NO:180 comprises glutamic acid (E) in EU position 356 and methionine (M) in EU position 358. In some embodiments, the antibodies provided herein comprise constant regions comprising D356/L358, E356/M358, D356/M358, or E356/L358 (EU numbering). However, a skilled person will recognize that the antibodies provide herein may comprise any suitable constant region and that the constant region sequences provided herein are for illustrative purposes.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

In some embodiments, the antibody inhibits the binding of LAG3 to one or more of its ligands. In some aspects, the antibody inhibits the binding of LAG3 to a ligand such as MHC class II.

The antibodies provided herein may be useful for the treatment of a variety of diseases and conditions including cancers. In particular, the antibodies provided herein may be useful for the treatment of cancers of epithelial origin.

2.1. CDR-H3 Sequences

In some embodiments, the antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a scFv-Fc sequence provided in SEQ ID No:145. In some aspects, the CDR-H3 sequence is a CDR-H3 sequence of a V_{H} sequence provided in SEQ ID NOs.:146-164.

In some embodiments, the antibody comprises a CDR-H3 sequence comprising, consisting of, or consisting essentially of a sequence selected from SEQ ID NOs:80-98. In some aspects, the antibody comprises a CDR-H3 sequence comprising, consisting of, or consisting essentially of SEQ ID NO:80. In some aspects, the antibody comprises a CDR-H3 sequence comprising, consisting of, or consisting essentially of SEQ ID NO:81. In some aspects, the antibody comprises a CDR-H3 sequence comprising, consisting of, or consisting essentially of SEQ ID NO:82. In some aspects, the antibody comprises a CDR-H3 sequence comprising, consisting of, or consisting essentially of SEQ ID NO:83. In some aspects, the antibody comprises a CDR-H3 sequence comprising, consisting of, or consisting essentially of SEQ ID NO:84. In some aspects, the antibody comprises a CDR-H3 sequence comprising, consisting of, or consisting essentially of SEQ ID NO:85. In some aspects, the antibody comprises a CDR-H3 sequence comprising, consisting of, or consisting essentially of SEQ ID NO:86. In some aspects, the antibody comprises a CDR-H3 sequence comprising, consisting of, or consisting essentially of SEQ ID NO:87. In some aspects, the antibody comprises a CDR-H3 sequence comprising, consisting of, or consisting essentially of SEQ ID NO:88. In some aspects, the antibody comprises a CDR-H3 sequence comprising, consisting of, or consisting essentially of SEQ ID NO:89. In some aspects, the antibody comprises a CDR-H3 sequence comprising, consisting of, or consisting essentially of SEQ ID NO:90. In some aspects, the antibody comprises a CDR-H3 sequence comprising, consisting of, or consisting essentially of SEQ ID NO:91. In some aspects, the antibody comprises a CDR-H3 sequence comprising, consisting of, or consisting essentially of SEQ ID NO:92. In some aspects, the antibody comprises a CDR-H3 sequence comprising, consisting of, or consisting essentially of SEQ ID NO:93. In some aspects, the antibody comprises a CDR-H3 sequence comprising, consisting of, or consisting essentially of SEQ ID NO:94. In some aspects, the antibody comprises a CDR-H3 sequence comprising, consisting of, or consisting essentially of SEQ ID NO:95. In some aspects, the antibody comprises a CDR-H3 sequence comprising, consisting of, or consisting essentially of SEQ ID NO:96. In some aspects, the antibody comprises a CDR-H3 sequence comprising, consisting of, or consisting essentially of SEQ ID NO:97. In some aspects, the antibody comprises a CDR-H3 sequence comprising, consisting of, or consisting essentially of SEQ ID NO:98.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least about 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-H3 sequence does not comprise, consist of, or consist essentially of SEQ ID NO:195.

2.2. V_{H} Sequences Comprising Illustrative CDRs

In some embodiments, the antibody comprises a V_{H} sequence comprising one or more CDR-H sequences comprising, consisting of, or consisting essentially of one or more illustrative CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 146-164.

2.2.1. V_{H} Sequences Comprising Illustrative Kabat CDRs

In some embodiments, the antibody comprises a V_{H} sequence comprising one or more Kabat CDR-H sequences comprising, consisting of, or consisting essentially of one or more illustrative Kabat CDR-H sequences provided in this disclosure, and variants thereof.

2.2.1.1. Kabat CDR-H3

In some embodiments, the antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a scFv-Fc sequence provided in SEQ ID NO.:145. In some aspects, the Kabat CDR-H3 sequence is a Kabat CDR-H3 sequence of a V_{H} sequence provided in SEQ ID NOs.:146-164.

In some embodiments, the antibody comprises a V_{H} sequence comprising a Kabat CDR-H3 sequence comprising, consisting of, or consisting essentially of a sequence selected from SEQ ID Nos:80-98. In some aspects, the antibody comprises a V_{H} sequence comprising a Kabat CDR-H3 sequence comprising, consisting of, or consisting essentially of SEQ ID NO:80. In some aspects, the antibody comprises a V_{H} sequence comprising a Kabat CDR-H3 sequence comprising, consisting of, or consisting essentially of SEQ ID NO:81. In some aspects, the antibody comprises a V_{H} sequence comprising a Kabat CDR-H3 sequence comprising, consisting of, or consisting essentially of SEQ ID NO:82. In some aspects, the antibody comprises a V_{H} sequence comprising a Kabat CDR-H3 sequence comprising, consisting of, or consisting essentially of SEQ ID NO:83. In some aspects, the antibody comprises a V_{H} sequence comprising a Kabat CDR-H3 sequence comprising, consisting of, or consisting essentially of SEQ ID NO:84. In some aspects, the antibody comprises a V_{H} sequence comprising a Kabat CDR-H3 sequence comprising, consisting of, or consisting essentially of SEQ ID NO:85. In some aspects, the antibody comprises a V_{H} sequence comprising a Kabat CDR-H3 sequence comprising, consisting of, or consisting essentially of SEQ ID NO:86. In some aspects, the antibody comprises a V_{H} sequence comprising a Kabat CDR-H3 sequence comprising, consisting of, or consisting essentially of SEQ ID NO:87. In some aspects, the antibody comprises a V_{H} sequence comprising a Kabat CDR-H3 sequence comprising, consisting of, or consisting essentially of SEQ ID NO:88. In some aspects, the antibody comprises a V_{H} sequence comprising a Kabat CDR-H3 sequence comprising, consisting of, or consisting essentially of SEQ ID NO:89. In some aspects, the antibody comprises a V_{H} sequence comprising a Kabat CDR-H3 sequence comprising, consisting of, or consisting essentially of SEQ ID NO:90. In some aspects, the antibody comprises a V_{H} sequence comprising a Kabat CDR-H3 sequence comprising, consisting of, or consisting essentially of SEQ ID NO:91. In some aspects, the antibody comprises a V_{H} sequence comprising a Kabat CDR-H3 sequence comprising, consisting of, or consisting essentially of SEQ ID NO:92. In some aspects, the antibody comprises a V_{H} sequence comprising a Kabat CDR-H3 sequence comprising, consisting of, or consisting essentially of SEQ ID NO:93. In some aspects, the antibody comprises a V_{H} sequence comprising a Kabat CDR-H3 sequence comprising, consisting of, or consisting essentially of SEQ ID NO:94. In some aspects, the antibody comprises a V_{H} sequence comprising a Kabat CDR-H3 sequence comprising, consisting of, or consisting essentially of SEQ ID NO:95. In some aspects, the antibody comprises a V_{H} sequence comprising a Kabat CDR-H3 sequence comprising, consisting of, or consisting essentially of SEQ ID NO:96. In some aspects, the antibody comprises a V_{H} sequence comprising a Kabat CDR-H3 sequence comprising, consisting of, or consisting essentially of SEQ ID NO:97. In some aspects, the antibody comprises a V_{H} sequence comprising a Kabat CDR-H3 sequence comprising, consisting of, or consisting essentially of SEQ ID NO:98.

2.2.1.2. Kabat CDR-H2

In some embodiments, the antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of an scFv-Fc sequence provided in SEQ ID NO.:145. In some aspects, the Kabat CDR-H2 sequence is a Kabat CDR-H2 sequence of a V_{H} sequence provided in SEQ ID NOs.:146-164.

In some embodiments, the antibody comprises a V_{H} sequence comprising a Kabat CDR-H2 sequence comprising, consisting of, or consisting essentially of a sequence selected from SEQ ID NOs:61-79. In some aspects, the antibody comprises a V_{H} sequence comprising a Kabat CDR-H2 sequence comprising, consisting of, or consisting essentially of SEQ ID NO:61. In some aspects, the antibody comprises a V_{H} sequence comprising a Kabat CDR-H2 sequence comprising, consisting of, or consisting essentially of SEQ ID NO:62. In some aspects, the antibody comprises a V_{H} sequence comprising a Kabat CDR-H2 sequence comprising, consisting of, or consisting essentially of SEQ ID NO:63. In some aspects, the antibody comprises a V_{H} sequence comprising a Kabat CDR-H2 sequence comprising, consisting of, or consisting essentially of SEQ ID NO:64. In some aspects, the antibody comprises a V_{H} sequence comprising a Kabat CDR-H2 sequence comprising, consisting of, or consisting essentially of SEQ ID NO:65. In some aspects, the antibody comprises a V_{H} sequence comprising a Kabat CDR-H2 sequence comprising, consisting of, or consisting essentially of SEQ ID NO:66. In some aspects, the antibody comprises a V_{H} sequence comprising a Kabat CDR-H2 sequence comprising, consisting of, or consisting essentially of SEQ ID NO:67. In some aspects, the antibody comprises a V_{H} sequence comprising a Kabat CDR-H2 sequence comprising, consisting of, or consisting essentially of SEQ ID NO:68. In some aspects, the antibody comprises a V_{H} sequence comprising a Kabat CDR-H2 sequence comprising, consisting of, or consisting essentially of SEQ ID NO:69. In some aspects, the antibody comprises a V_{H} sequence comprising a Kabat CDR-H2 sequence comprising, consisting of, or consisting essentially of SEQ ID NO:70. In some aspects, the antibody comprises a V_{H} sequence comprising a Kabat CDR-H2 sequence comprising, consisting of, or consisting essentially of SEQ ID NO:71. In some aspects, the antibody comprises a V_{H} sequence comprising a Kabat CDR-H2 sequence comprising, consisting of, or consisting essentially of SEQ ID NO:72. In some aspects, the antibody comprises a V_{H} sequence comprising a Kabat CDR-H2 sequence comprising, consisting of, or consisting essentially of SEQ ID NO:73. In some aspects, the antibody comprises a V_{H} sequence comprising a Kabat CDR-H2 sequence comprising, consisting of, or consisting essentially of SEQ ID NO:74. In some aspects, the antibody comprises a V_{H} sequence comprising a Kabat CDR-H2 sequence comprising, consisting of, or consisting essentially of SEQ ID NO:75. In some aspects, the antibody comprises a V_{H} sequence comprising a Kabat CDR-H2 sequence comprising, consisting of, or consisting essentially of SEQ ID NO:76. In some aspects, the antibody comprises a V_{H} sequence comprising a Kabat CDR-H2 sequence comprising, consisting of, or consisting essentially of SEQ ID NO:77. In some aspects, the antibody comprises a V_{H} sequence comprising a Kabat CDR-H2 sequence comprising, consisting of, or consisting essentially of SEQ ID NO:78. In some aspects, the antibody comprises a V_{H} sequence comprising a Kabat CDR-H2 sequence comprising, consisting of, or consisting essentially of SEQ ID NO:79.

2.2.1.3. Kabat CDR-H1

In some embodiments, the antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of an scFv-Fc sequence provided in SEQ ID NO.:145. In some aspects, the Kabat CDR-H1 sequence is a Kabat CDR-H1 sequence of a V_{H} sequence provided in SEQ ID NOs.:146-164.

In some embodiments, the antibody comprises a V_{H} sequence comprising a Kabat CDR-H1 sequence comprising, consisting of, or consisting essentially of a sequence selected from SEQ ID NOs:23-41. In some aspects, the antibody comprises a V_{H} sequence comprising a Kabat CDR-H1 sequence comprising, consisting of, or consisting essentially of SEQ ID NO:23. In some aspects, the antibody comprises a V_{H} sequence comprising a Kabat CDR-H1 sequence comprising, consisting of, or consisting essentially of SEQ ID NO:24. In some aspects, the antibody comprises a V_{H} sequence comprising a Kabat CDR-H1 sequence comprising, consisting of, or consisting essentially of SEQ ID NO:25. In some aspects, the antibody comprises a V_{H} sequence comprising a Kabat CDR-H1 sequence comprising, consisting of, or consisting essentially of SEQ ID NO:26. In some aspects, the antibody comprises a V_{H} sequence comprising a Kabat CDR-H1 sequence comprising, consisting of, or consisting essentially of SEQ ID NO:27. In some aspects, the antibody comprises a V_{H} sequence comprising a Kabat CDR-H1 sequence comprising, consisting of, or consisting essentially of SEQ ID NO:28. In some aspects, the antibody comprises a V_{H} sequence comprising a Kabat CDR-H1 sequence comprising, consisting of, or consisting essentially of SEQ ID NO:29. In some aspects, the antibody comprises a V_{H} sequence comprising a Kabat CDR-H1 sequence comprising, consisting of, or consisting essentially of SEQ ID NO:30. In some aspects, the antibody comprises a V_{H} sequence comprising a Kabat CDR-H1 sequence comprising, consisting of, or consisting essentially of SEQ ID NO:31. In some aspects, the antibody comprises a V_{H} sequence comprising a Kabat CDR-H1 sequence comprising, consisting of, or consisting essentially of SEQ ID NO:32. In some aspects, the antibody comprises a V_{H} sequence comprising a Kabat CDR-H1 sequence comprising, consisting of, or consisting essentially of SEQ ID NO:33. In some aspects, the antibody comprises a V_{H} sequence comprising a Kabat CDR-H1 sequence comprising, consisting of, or consisting essentially of SEQ ID NO:34. In some aspects, the antibody comprises a V_{H} sequence comprising a Kabat CDR-H1 sequence comprising, consisting of, or consisting essentially of SEQ ID NO:35. In some aspects, the antibody comprises a V_{H} sequence comprising a Kabat CDR-H1 sequence comprising, consisting of, or consisting essentially of SEQ ID NO:36. In some aspects, the antibody comprises a V_{H} sequence comprising a Kabat CDR-H1 sequence comprising, consisting of, or consisting essentially of SEQ ID NO:37. In some aspects, the antibody comprises a V_{H} sequence comprising a Kabat CDR-H1 sequence comprising, consisting of, or consisting essentially of SEQ ID NO:38. In some aspects, the antibody comprises a V_{H} sequence comprising a Kabat CDR-H1 sequence comprising, consisting of, or consisting essentially of SEQ ID NO:39. In some aspects, the antibody comprises a V_{H} sequence comprising a Kabat CDR-H1 sequence comprising, consisting of, or consisting essentially of SEQ ID NO:40. In some aspects, the antibody comprises a V_{H} sequence comprising a Kabat CDR-H1 sequence comprising, consisting of, or consisting essentially of SEQ ID NO:41.

2.2.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID Nos:80-98, and a Kabat CDR-H2 sequence comprising, consisting of, or consisting essentially of a sequence selected from SEQ ID NOs:61-79. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs:146-164.

2.2.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs:80-98, and a Kabat CDR-H1 sequence comprising, consisting of, or consisting essentially of a sequence selected from SEQ ID NOs:23-41. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs:146-164.

2.2.1.6. Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs:23-41 and a Kabat CDR-H2 sequence comprising, consisting of, or consisting essentially of a sequence selected from SEQ ID NOs:61-79. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs:146-164.

2.2.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID Nos:23-41, a Kabat CDR-H2 sequence comprising, consisting of, or consisting essentially of a sequence selected from SEQ ID NOs:61-79, and a Kabat CDR-H3 sequence comprising, consisting of, or consisting essentially of a sequence selected from SEQ ID NOs:80-98. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs:146-164.

2.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least about 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least about 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least about 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

2.2.1.9. Excluded V_{H} Sequences Comprising Kabat CDRs

In some embodiments, the V_{H} sequences provided herein do not comprise certain Kabat CDR-H3, CDR-H2, and/or CDR-H1 sequences. In some aspects, the Kabat CDR-H3 sequence does not comprise, consist of, or consist essentially of a sequence selected from SEQ ID NO:195. In some aspects, the Kabat CDR-H2 sequence does not comprise, consist of, or consist essentially of a sequence selected from SEQ ID NO:194. In some aspects, the Kabat CDR-H1 sequence does not comprise, consist of, or consist essentially of a sequence selected from SEQ ID NO:192.

2.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

2.2.2.1. Chothia CDR-H3

In some embodiments, the antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of an scFv-Fc sequence provided in SEQ ID NO:145. In some aspects, the Chothia CDR-H3 sequence is a Chothia CDR-H3 sequence of a V_{H} sequence provided in SEQ ID NOs.:146-164.

In some embodiments, the antibody comprises a V_{H} sequence comprising a Chothia CDR-H3 sequence comprising, consisting of, or consisting essentially of a sequence selected from SEQ ID Nos:80-98. In some aspects, the antibody comprises a V_{H} sequence comprising a Chothia CDR-H3 sequence comprising, consisting of, or consisting essentially of SEQ ID NO:80. In some aspects, the antibody comprises a V_{H} sequence comprising a Chothia CDR-H3 sequence comprising, consisting of, or consisting essentially of SEQ ID NO:81. In some aspects, the antibody comprises a V_{H} sequence comprising a Chothia CDR-H3 sequence comprising, consisting of, or consisting essentially of SEQ ID NO:82. In some aspects, the antibody comprises a V_{H} sequence comprising a Chothia CDR-H3 sequence comprising, consisting of, or consisting essentially of SEQ ID NO:83. In some aspects, the antibody comprises a V_{H} sequence comprising a Chothia CDR-H3 sequence comprising, consisting of, or consisting essentially of SEQ ID NO:84. In some aspects, the antibody comprises a V_{H} sequence comprising a Chothia CDR-H3 sequence comprising, consisting of, or consisting essentially of SEQ ID NO:85. In some aspects, the antibody comprises a V_{H} sequence comprising a Chothia CDR-H3 sequence comprising, consisting of, or consisting essentially of SEQ ID NO:86. In some aspects, the antibody comprises a V_{H} sequence comprising a Chothia CDR-H3 sequence comprising, consisting of, or consisting essentially of SEQ ID NO:87. In some aspects, the antibody comprises a V_{H} sequence comprising a Chothia CDR-H3 sequence comprising, consisting of, or consisting essentially of SEQ ID NO:88. In some aspects, the antibody comprises a V_{H} sequence comprising a Chothia CDR-H3 sequence comprising, consisting of, or consisting essentially of SEQ ID NO:89. In some aspects, the antibody comprises a V_{H} sequence comprising a Chothia CDR-H3 sequence comprising, consisting of, or consisting essentially of SEQ ID NO:90. In some aspects, the antibody comprises a V_{H} sequence comprising a Chothia CDR-H3 sequence comprising, consisting of, or consisting essentially of SEQ ID NO:91. In some aspects, the antibody comprises a V_{H} sequence comprising a Chothia CDR-H3 sequence comprising, consisting of, or consisting essentially of SEQ ID NO:92. In some aspects, the antibody comprises a V_{H} sequence comprising a Chothia CDR-H3 sequence comprising, consisting of, or consisting essentially of SEQ ID NO:93. In some aspects, the antibody comprises a V_{H} sequence comprising a Chothia CDR-H3 sequence comprising, consisting of, or consisting essentially of SEQ ID NO:94. In some aspects, the antibody comprises a V_{H} sequence comprising a Chothia CDR-H3 sequence comprising, consisting of, or consisting essentially of SEQ ID NO:95. In some aspects, the antibody comprises a V_{H} sequence comprising a Chothia CDR-H3 sequence comprising, consisting of, or consisting essentially of SEQ ID NO:96. In some aspects, the antibody comprises a V_{H} sequence comprising a Chothia CDR-H3 sequence comprising, consisting of, or consisting essentially of SEQ ID NO:97. In some aspects, the antibody comprises a V_{H} sequence comprising a Chothia CDR-H3 sequence comprising, consisting of, or consisting essentially of SEQ ID NO:98.

2.2.2.2. Chothia CDR-H2

In some embodiments, the antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of an scFv-Fc sequence provided in SEQ ID NO:145. In some aspects, the Chothia CDR-H2 sequence is a Chothia CDR-H2 sequence of a V_{H} sequence provided in SEQ ID NOs.:146-164.

In some embodiments, the antibody comprises a V_{H} sequence comprising a Chothia CDR-H2 sequence comprising, consisting of, or consisting essentially of a sequence selected from SEQ ID Nos:42-60. In some aspects, the antibody comprises a V_{H} sequence comprising a Chothia CDR-H2 sequence comprising, consisting of, or consisting essentially of SEQ ID NO:42. In some aspects, the antibody comprises a V_{H} sequence comprising a Chothia CDR-H2 sequence comprising, consisting of, or consisting essentially of SEQ ID NO:43. In some aspects, the antibody comprises a V_{H} sequence comprising a Chothia CDR-H2 sequence comprising, consisting of, or consisting essentially of SEQ ID NO:44. In some aspects, the antibody comprises a V_{H} sequence comprising a Chothia CDR-H2 sequence comprising, consisting of, or consisting essentially of SEQ ID NO:45. In some aspects, the antibody comprises a V_{H} sequence comprising a Chothia CDR-H2 sequence comprising, consisting of, or consisting essentially of SEQ ID NO:46. In some aspects, the antibody comprises a V_{H} sequence comprising a Chothia CDR-H2 sequence comprising, consisting of, or consisting essentially of SEQ ID NO:47. In some aspects, the antibody comprises a V_{H} sequence comprising a Chothia CDR-H2 sequence comprising, consisting of, or consisting essentially of SEQ ID NO:48. In some aspects, the antibody comprises a V_{H} sequence comprising a Chothia CDR-H2 sequence comprising, consisting of, or consisting essentially of SEQ ID NO:49. In some aspects, the antibody comprises a V_{H} sequence comprising a Chothia CDR-H2 sequence comprising, consisting of, or consisting essentially of SEQ ID NO:50. In some aspects, the antibody comprises a V_{H} sequence comprising a Chothia CDR-H2 sequence comprising, consisting of, or consisting essentially of SEQ ID NO:51. In some aspects, the antibody comprises a V_{H} sequence comprising a Chothia CDR-H2 sequence comprising, consisting of, or consisting essentially of SEQ ID NO:52. In some aspects, the antibody comprises a V_{H} sequence comprising a Chothia CDR-H2 sequence comprising, consisting of, or consisting essentially of SEQ ID NO:53. In some aspects, the antibody comprises a V_{H} sequence comprising a Chothia CDR-H2 sequence comprising, consisting of, or consisting essentially of SEQ ID NO:54. In some aspects, the antibody comprises a V_{H} sequence comprising a Chothia CDR-H2 sequence comprising, consisting of, or consisting essentially of SEQ ID NO:55. In some aspects, the antibody comprises a V_{H} sequence comprising a Chothia CDR-H2 sequence comprising, consisting of, or consisting essentially of SEQ ID NO:56. In some aspects, the antibody comprises a V_{H} sequence comprising a Chothia CDR-H2 sequence comprising, consisting of, or consisting essentially of SEQ ID NO:57. In some aspects, the antibody comprises a V_{H} sequence comprising a Chothia CDR-H2 sequence comprising, consisting of, or consisting essentially of SEQ ID NO:58. In some aspects, the antibody comprises a V_{H} sequence comprising a Chothia CDR-H2 sequence comprising, consisting of, or consisting essentially of SEQ ID NO:59. In some aspects, the antibody comprises a V_{H} sequence comprising a Chothia CDR-H2 sequence comprising, consisting of, or consisting essentially of SEQ ID NO:60.

2.2.2.3. Chothia CDR-H1

In some embodiments, the antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of an scFv-Fc sequence provided in SEQ ID NO:145. In some aspects, the Chothia CDR-H1 sequence is a Chothia CDR-H1 sequence of a V_{H} sequence provided in SEQ ID NOs.:146-164.

In some embodiments, the antibody comprises a V_{H} sequence comprising a Chothia CDR-H1 sequence comprising, consisting of, or consisting essentially of a sequence selected from SEQ ID NOs:4-22. In some aspects, the antibody comprises a V_{H} sequence comprising a Chothia CDR-H1 sequence comprising, consisting of, or consisting essentially of SEQ ID NO:4. In some aspects, the antibody comprises a V_{H} sequence comprising a Chothia CDR-H1 sequence comprising, consisting of, or consisting essentially of SEQ ID NO:5. In some aspects, the antibody comprises a V_{H} sequence comprising a Chothia CDR-H1 sequence comprising, consisting of, or consisting essentially of SEQ ID NO:6. In some aspects, the antibody comprises a V_{H} sequence comprising a Chothia CDR-H1 sequence comprising, consisting of, or consisting essentially of SEQ ID NO:7. In some aspects, the antibody comprises a V_{H} sequence comprising a Chothia CDR-H1 sequence comprising, consisting of, or consisting essentially of SEQ ID NO:8. In some aspects, the antibody comprises a V_{H} sequence comprising a Chothia CDR-H1 sequence comprising, consisting of, or consisting essentially of SEQ ID NO:9. In some aspects, the antibody comprises a V_{H} sequence comprising a Chothia CDR-H1 sequence comprising, consisting of, or consisting essentially of SEQ ID NO:10. In some aspects, the antibody comprises a V_{H} sequence comprising a Chothia CDR-H1 sequence comprising, consisting of, or consisting essentially of SEQ ID NO:11. In some aspects, the antibody comprises a V_{H} sequence comprising a Chothia CDR-H1 sequence comprising, consisting of, or consisting essentially of SEQ ID NO:12. In some aspects, the antibody comprises a V_{H} sequence comprising a Chothia CDR-H1 sequence comprising, consisting of, or consisting essentially of SEQ ID NO:13. In some aspects, the antibody comprises a V_{H} sequence comprising a Chothia CDR-H1 sequence comprising, consisting of, or consisting essentially of SEQ ID NO:14. In some aspects, the antibody comprises a V_{H} sequence comprising a Chothia CDR-H1 sequence comprising, consisting of, or consisting essentially of SEQ ID NO:15. In some aspects, the antibody comprises a V_{H} sequence comprising a Chothia CDR-H1 sequence comprising, consisting of, or consisting essentially of SEQ ID NO:16. In some aspects, the antibody comprises a V_{H} sequence comprising a Chothia CDR-H1 sequence comprising, consisting of, or consisting essentially of SEQ ID NO:17. In some aspects, the antibody comprises a V_{H} sequence comprising a Chothia CDR-H1 sequence comprising, consisting of, or consisting essentially of SEQ ID NO:18. In some aspects, the antibody comprises a V_{H} sequence comprising a Chothia CDR-H1 sequence comprising, consisting of, or consisting essentially of SEQ ID NO:19. In some aspects, the antibody comprises a V_{H} sequence comprising a Chothia CDR-H1 sequence comprising, consisting of, or consisting essentially of SEQ ID NO:20. In some aspects, the antibody comprises a V_{H} sequence comprising a Chothia CDR-H1 sequence comprising, consisting of, or consisting essentially of SEQ ID NO:21. In some aspects, the antibody comprises a V_{H} sequence comprising a Chothia CDR-H1 sequence comprising, consisting of, or consisting essentially of SEQ ID NO:22.

2.2.2.4. Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOs:80-98, and a Chothia CDR-H2 sequence comprising, consisting of, or consisting essentially of a sequence selected from SEQ ID NOs:42-60. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs:146-164.

2.2.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs:80-98, and a Chothia CDR-H1 sequence comprising, consisting of, or consisting essentially of a sequence selected from SEQ ID NOs: 4-22. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs:146-164.

2.2.2.6. Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs:4-22 and a Chothia CDR-H2 sequence comprising, consisting of, or consisting essentially of a sequence selected from SEQ ID NOs:42-60. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs:146-164.

2.2.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs:4-22, a Chothia CDR-H2 sequence comprising, consisting of, or consisting essentially of a sequence selected from SEQ ID NOs:42-60, and a Chothia CDR-H3 sequence comprising, consisting of, or consisting essentially of a sequence selected from SEQ ID NOs.:80-98. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs:146-164.

2.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least about 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least about 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least about 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

2.2.2.9. Excluded V_{H} Sequences Comprising Chothia CDRs

In some embodiments, the V_{H} sequences provided herein do not comprise certain Chothia CDR-H3, CDR-H2, and/or CDR-H1 sequences. In some aspects, the Chothia CDR-H3 sequence does not comprise, consist of, or consist essentially of a sequence selected from SEQ ID NO:195. In some aspects, the Chothia CDR-H2 sequence does not comprise, consist of, or consist essentially of a sequence selected from SEQ ID NO:193. In some aspects, the Chothia CDR-H1 sequence does not comprise, consist of, or consist essentially of a sequence selected from SEQ ID NO:191.

2.3. V_{H} Sequences

In some embodiments, the antibody comprises, consists of, or consists essentially of a V_{H} sequence of an scFv-Fc sequence provided in SEQ ID NOs.:145. In some embodiments, the antibody comprises, consists of, or consists essentially of a V_{H} sequence provided in SEQ ID NOs.: 146-164.

In some embodiments, the antibody comprises a V_{H} sequence comprising, consisting of, or consisting essentially of a sequence selected from SEQ ID NOs:146-164. In some aspects, the antibody comprises a V_{H} sequence comprising, consisting of, or consisting essentially of SEQ ID NO:146. In some aspects, the antibody comprises a V_{H} sequence comprising, consisting of, or consisting essentially of SEQ ID NO:147. In some aspects, the antibody comprises a V_{H} sequence comprising, consisting of, or consisting essentially of SEQ ID NO:148. In some aspects, the antibody comprises a V_{H} sequence comprising, consisting of, or consisting essentially of SEQ ID NO:149. In some aspects, the antibody comprises a V_{H} sequence comprising, consisting of, or consisting essentially of SEQ ID NO:150. In some aspects, the antibody comprises a V_{H} sequence comprising, consisting of, or consisting essentially of SEQ ID NO:151. In some aspects, the antibody comprises a V_{H} sequence comprising, consisting of, or consisting essentially of SEQ ID NO:152. In some aspects, the antibody comprises a V_{H} sequence comprising, consisting of, or consisting essentially of SEQ ID NO:153. In some aspects, the antibody comprises a V_{H} sequence comprising, consisting of, or consisting essentially of SEQ ID NO:154. In some aspects, the antibody comprises a V_{H} sequence comprising, consisting of, or consisting essentially of SEQ ID NO:155. In some aspects, the antibody comprises a V_{H} sequence comprising, consisting of, or consisting essentially of SEQ ID NO:156. In some aspects, the antibody comprises a V_{H} sequence comprising, consisting of, or consisting essentially of SEQ ID NO:157. In some aspects, the antibody comprises a V_{H} sequence comprising, consisting of, or consisting essentially of SEQ ID NO:158. In some aspects, the antibody comprises a V_{H} sequence comprising, consisting of, or consisting essentially of SEQ ID NO:159. In some aspects, the antibody comprises a V_{H} sequence comprising, consisting of, or consisting essentially of SEQ ID NO:160. In some aspects, the antibody comprises a V_{H} sequence comprising, consisting of, or consisting essentially of SEQ ID NO:161. In some aspects, the antibody comprises a V_{H} sequence comprising, consisting of, or consisting essentially of SEQ ID NO:162. In some aspects, the antibody comprises a V_{H} sequence comprising, consisting of, or consisting essentially of SEQ ID NO:163. In some aspects, the antibody comprises a V_{H} sequence comprising, consisting of, or consisting essentially of SEQ ID NO:164.

2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least about 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.3.2. Excluded V_{H} Sequences

In some embodiments, the V_{H} sequences provided herein do not comprise certain V_{H} sequences. In some aspects, the V_{H} sequence does not comprise, consist of, or consist essentially of a sequence selected from SEQ ID NO:199.

2.4. CDR-L3 Sequences

In some embodiments, the antibody comprises a CDR-L3 sequence comprising, consisting of, or consisting essentially of a CDR-L3 sequence of an illustrative antibody or V_{L} sequence provided herein. In some aspects, the CDR-L3 sequence is a CDR-L3 sequence of an scFv-Fc sequence provided in SEQ ID NO:145. In some aspects, the CDR-L3 sequence is a CDR-L3 sequence of a V_{L} sequence provided in SEQ ID NOs.:165-179.

In some embodiments, the antibody comprises a CDR-L3 sequence comprising, consisting of, or consisting essentially of a sequence selected from SEQ ID NOs:130-144. In some aspects, the antibody comprises a CDR-L3 sequence comprising, consisting of, or consisting essentially of SEQ ID NO:130. In some aspects, the antibody comprises a CDR-L3 sequence comprising, consisting of, or consisting essentially of SEQ ID NO:131. In some aspects, the antibody comprises a CDR-L3 sequence comprising, consisting of, or consisting essentially of SEQ ID NO:132. In some aspects, the antibody comprises a CDR-L3 sequence comprising, consisting of, or consisting essentially of SEQ ID NO:133. In some aspects, the antibody comprises a CDR-L3 sequence comprising, consisting of, or consisting essentially of SEQ ID NO:134. In some aspects, the antibody comprises a CDR-L3 sequence comprising, consisting of, or consisting essentially of SEQ ID NO:135. In some aspects, the antibody comprises a CDR-L3 sequence comprising, consisting of, or consisting essentially of SEQ ID NO:136. In some aspects, the antibody comprises a CDR-L3 sequence comprising, consisting of, or consisting essentially of SEQ ID NO:137. In some aspects, the antibody comprises a CDR-L3 sequence comprising, consisting of, or consisting essentially of SEQ ID NO:138. In some aspects, the antibody comprises a CDR-L3 sequence comprising, consisting of, or consisting essentially of SEQ ID NO:139. In some aspects, the antibody comprises a CDR-L3 sequence comprising, consisting of, or consisting essentially of SEQ ID NO:140. In some aspects, the antibody comprises a CDR-L3 sequence comprising, consisting of, or consisting essentially of SEQ ID NO:141. In some aspects, the antibody comprises a CDR-L3 sequence comprising, consisting of, or consisting essentially of SEQ ID NO:142. In some aspects, the antibody comprises a CDR-L3 sequence comprising, consisting of, or consisting essentially of SEQ ID NO:143. In some aspects, the antibody comprises a CDR-L3 sequence comprising, consisting of, or consisting essentially of SEQ ID NO:144.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least about 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L3 sequence does not comprise, consist of, or consist essentially of a sequence selected from SEQ ID NO:198.

2.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

2.5.1. CDR-L3

In some embodiments, the antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of an scFv-Fc sequence provided in SEQ ID NO:145. In some aspects, the CDR-L3 sequence is a CDR-L3 sequence of a V_{L} sequence provided in SEQ ID NOs.: 165-179.

In some embodiments, the antibody comprises a V_{L} sequence comprising a CDR-L3 sequence comprising, consisting of, or consisting essentially of a sequence selected from SEQ ID NOs:130-144. In some aspects, the antibody comprises a V_{L} sequence comprising a CDR-L3 sequence comprising, consisting of, or consisting essentially of SEQ ID NO:130. In some aspects, the antibody comprises a V_{L} sequence comprising a CDR-L3 sequence comprising, consisting of, or consisting essentially of SEQ ID NO:131. In some aspects, the antibody comprises a V_{L} sequence comprising a CDR-L3 sequence comprising, consisting of, or consisting essentially of SEQ ID NO:132. In some aspects, the antibody comprises a V_{L} sequence comprising a CDR-L3 sequence comprising, consisting of, or consisting essentially of SEQ ID NO:133. In some aspects, the antibody comprises a V_{L} sequence comprising a CDR-L3 sequence comprising, consisting of, or consisting essentially of SEQ ID NO:134. In some aspects, the antibody comprises a V_{L} sequence comprising a CDR-L3 sequence comprising, consisting of, or consisting essentially of SEQ ID NO:135. In some aspects, the antibody comprises a V_{L} sequence comprising a CDR-L3 sequence comprising, consisting of, or consisting essentially of SEQ ID NO:136. In some aspects, the antibody comprises a V_{L} sequence comprising a CDR-L3 sequence comprising, consisting of, or consisting essentially of SEQ ID NO:137. In some aspects, the antibody comprises a V_{L} sequence comprising a CDR-L3 sequence comprising, consisting of, or consisting essentially of SEQ ID NO:138. In some aspects, the antibody comprises a V_{L} sequence comprising a CDR-L3 sequence comprising, consisting of, or consisting essentially of SEQ ID NO:139. In some aspects, the antibody comprises a V_{L} sequence comprising a CDR-L3 sequence comprising, consisting of, or consisting essentially of SEQ ID NO:140. In some aspects, the antibody comprises a V_{L} sequence comprising a CDR-L3 sequence comprising, consisting of, or consisting essentially of SEQ ID NO:141. In some aspects, the antibody comprises a V_{L} sequence comprising a CDR-L3 sequence comprising, consisting of, or consisting essentially of SEQ ID NO:142. In some aspects, the antibody comprises a V_{L} sequence comprising a CDR-L3 sequence comprising, consisting of, or consisting essentially of SEQ ID NO:143. In some aspects, the antibody comprises a V_{L} sequence comprising a CDR-L3 sequence comprising, consisting of, or consisting essentially of SEQ ID NO:144.

2.5.2. CDR-L2

In some embodiments, the antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of an scFv-Fc sequence provided in SEQ ID NO:145. In some aspects, the CDR-L2 sequence is a CDR-L2 sequence of a V_{L} sequence provided in SEQ ID NOs.:165-179.

In some embodiments, the antibody comprises a V_{L} sequence comprising a CDR-L2 sequence comprising, consisting of, or consisting essentially of a sequence selected from SEQ ID NOs:115-129. In some aspects, the antibody comprises a V_{L} sequence comprising a CDR-L2 sequence comprising, consisting of, or consisting essentially of SEQ ID NO:115. In some aspects, the antibody comprises a V_{L} sequence comprising a CDR-L2 sequence comprising, consisting of, or consisting essentially of SEQ ID NO:116. In some aspects, the antibody comprises a V_{L} sequence comprising a CDR-L2 sequence comprising, consisting of, or consisting essentially of SEQ ID NO:117. In some aspects, the antibody comprises a V_{L} sequence comprising a CDR-L2 sequence comprising, consisting of, or consisting essentially of SEQ ID NO:118. In some aspects, the antibody comprises a V_{L} sequence comprising a CDR-L2 sequence comprising, consisting of, or consisting essentially of SEQ ID NO:119. In some aspects, the antibody comprises a V_{L} sequence comprising a CDR-L2 sequence comprising, consisting of, or consisting essentially of SEQ ID NO:120. In some aspects, the antibody comprises a V_{L} sequence comprising a CDR-L2 sequence comprising, consisting of, or consisting essentially of SEQ ID NO:121. In some aspects, the antibody comprises a V_{L} sequence comprising a CDR-L2 sequence comprising, consisting of, or consisting essentially of SEQ ID NO:122. In some aspects, the antibody comprises a V_{L} sequence comprising a CDR-L2 sequence comprising, consisting of, or consisting essentially of SEQ ID NO:123. In some aspects, the antibody comprises a V_{L} sequence comprising a CDR-L2 sequence comprising, consisting of, or consisting essentially of SEQ ID NO:124. In some aspects, the antibody comprises a V_{L} sequence comprising a CDR-L2 sequence comprising, consisting of, or consisting essentially of SEQ ID NO:125. In some aspects, the antibody comprises a V_{L} sequence comprising a CDR-L2 sequence comprising, consisting of, or consisting essentially of SEQ ID NO:126. In some aspects, the antibody comprises a V_{L} sequence comprising a CDR-L2 sequence comprising, consisting of, or consisting essentially of SEQ ID NO:127. In some aspects, the antibody comprises a V_{L} sequence comprising a CDR-L2 sequence comprising, consisting of, or consisting essentially of SEQ ID NO:128. In some aspects, the antibody comprises a V_{L} sequence comprising a CDR-L2 sequence comprising, consisting of, or consisting essentially of SEQ ID NO:129.

2.5.3. CDR-L1

In some embodiments, the antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of an scFv-Fc sequence provided in SEQ ID NOs.:145. In some aspects, the CDR-L1 sequence is a CDR-L1 sequence of a V_{L} sequence provided in SEQ ID NOs.: 165-179.

In some embodiments, the antibody comprises a V_{L} sequence comprising a CDR-L1 sequence comprising, consisting of, or consisting essentially of a sequence selected from SEQ ID NOs:100-114. In some aspects, the antibody comprises a V_{L} sequence comprising a CDR-L1 sequence comprising, consisting of, or consisting essentially of SEQ ID NO:100. In some aspects, the antibody comprises a V_{L} sequence comprising a CDR-L1 sequence comprising, consisting of, or consisting essentially of SEQ ID NO:101. In some aspects, the antibody comprises a V_{L} sequence comprising a CDR-L1 sequence comprising, consisting of, or consisting essentially of SEQ ID NO:102. In some aspects, the antibody comprises a V_{L} sequence comprising a CDR-L1 sequence comprising, consisting of, or consisting essentially of SEQ ID NO:103. In some aspects, the antibody comprises a V_{L} sequence comprising a CDR-L1 sequence comprising, consisting of, or consisting essentially of SEQ ID NO:104. In some aspects, the antibody comprises a V_{L} sequence comprising a CDR-L1 sequence comprising, consisting of, or consisting essentially of SEQ ID NO:105. In some aspects, the antibody comprises a V_{L} sequence comprising a CDR-L1 sequence comprising, consisting of, or consisting essentially of SEQ ID NO:106. In some aspects, the antibody comprises a V_{L} sequence comprising a CDR-L1 sequence comprising, consisting of, or consisting essentially of SEQ ID NO:107. In some aspects, the antibody comprises a V_{L} sequence comprising a CDR-L1 sequence comprising, consisting of, or consisting essentially of SEQ ID NO:108. In some aspects, the antibody comprises a V_{L} sequence comprising a CDR-L1 sequence comprising, consisting of, or consisting essentially of SEQ ID NO:109. In some aspects, the antibody comprises a V_{L} sequence comprising a CDR-L1 sequence comprising, consisting of, or consisting essentially of SEQ ID NO:110. In some aspects, the antibody comprises a V_{L} sequence comprising a CDR-L1 sequence comprising, consisting of, or consisting essentially of SEQ ID NO:111. In some aspects, the antibody comprises a V_{L} sequence comprising a CDR-L1 sequence comprising, consisting of, or consisting essentially of SEQ ID NO:112. In some aspects, the antibody comprises a V_{L} sequence comprising a CDR-L1 sequence comprising, consisting of, or consisting essentially of SEQ ID NO:113. In some aspects, the antibody comprises a V_{L} sequence comprising a CDR-L1 sequence comprising, consisting of, or consisting essentially of SEQ ID NO:114.

2.5.4. CDR-L3+CDR-L2

In some embodiments, the antibody comprises a V_{L} sequence comprising a CDR-L3 sequence comprising, consisting of, or consisting essentially of a sequence selected from SEQ ID NOs:130-144 and a CDR-L2 sequence comprising, consisting of, or consisting essentially of a sequence selected from SEQ ID NOs:115-129. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 165-179.

2.5.5. CDR-L3+CDR-L1

In some embodiments, the antibody comprises a V_{L} sequence comprising a CDR-L3 sequence comprising, consisting of, or consisting essentially of a sequence selected from SEQ ID NOs:130-144 and a CDR-L1 sequence comprising, consisting of, or consisting essentially of a sequence selected from SEQ ID NOs:100-114. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 165-179.

2.5.6. CDR-L1+CDR-L2

In some embodiments, the antibody comprises a V_{L} sequence comprising a CDR-L1 sequence comprising, consisting of, or consisting essentially of a sequence selected from SEQ ID NOs:100-114 and a CDR-L2 sequence comprising, consisting of, or consisting essentially of a sequence selected from SEQ ID NOs:115-129. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 165-179.

2.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs:100-114, a CDR-L2 sequence comprising, consisting of, or consisting essentially of a sequence selected from SEQ ID NOs:115-129, and a CDR-L3 sequence comprising, consisting of, or consisting essentially of a sequence selected from SEQ ID NOs:130-144. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs:165-179.

2.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least about 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least about 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least about 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

2.5.9. Excluded V_{L} Sequences Comprising CDR-Ls

In some embodiments, the V_{L} sequences provided herein do not comprise certain CDR-L3, CDR-L2, and/or CDR-L1 sequences. In some aspects, the CDR-L3 sequence does not comprise, consist of, or consist essentially of a sequence selected from SEQ ID NO:198. In some aspects, the CDR-L2 sequence does not comprise, consist of, or consist essentially of a sequence selected from SEQ ID NO:197. In some aspects, the CDR-L1 sequence does not comprise, consist of, or consist essentially of a sequence selected from SEQ ID NO:196.

2.6. V_{L} Sequences

In some embodiments, the antibody comprises, consists of, or consists essentially of a V_{L} sequence of an scFv-Fc sequence provided in SEQ ID NOs.:145. In some embodiments, the antibody comprises, consists of, or consists essentially of a V_{L} sequence provided in SEQ ID NOs.:165-179.

In some embodiments, the antibody comprises a V_{L} sequence comprising, consisting of, or consisting essentially of a sequence selected from SEQ ID NOs:165-179. In some aspects, the antibody comprises a V_{L} sequence comprising, consisting of, or consisting essentially of SEQ ID NO:165. In some aspects, the antibody comprises a V_{L} sequence comprising, consisting of, or consisting essentially of SEQ ID NO:166. In some aspects, the antibody comprises a V_{L} sequence comprising, consisting of, or consisting essentially of SEQ ID NO:167. In some aspects, the antibody comprises a V_{L} sequence comprising, consisting of, or consisting essentially of SEQ ID NO:168. In some aspects, the antibody comprises a V_{L} sequence comprising, consisting of, or consisting essentially of SEQ ID NO:169. In some aspects, the antibody comprises a V_{L} sequence comprising, consisting of, or consisting essentially of SEQ ID NO:170. In some aspects, the antibody comprises a V_{L} sequence comprising, consisting of, or consisting essentially of SEQ ID NO:171. In some aspects, the antibody comprises a V_{L} sequence comprising, consisting of, or consisting essentially of SEQ ID NO:172. In some aspects, the antibody comprises a V_{L} sequence comprising, consisting of, or consisting essentially of SEQ ID NO:173. In some aspects, the antibody comprises a V_{L} sequence comprising, consisting of, or consisting essentially of SEQ ID NO:174. In some aspects, the antibody comprises a V_{L} sequence comprising, consisting of, or consisting essentially of SEQ ID NO:175. In some aspects, the antibody comprises a V_{L} sequence comprising, consisting of, or consisting essentially of SEQ ID NO:176. In some aspects, the antibody comprises a V_{L} sequence comprising, consisting of, or consisting essentially of SEQ ID NO:177. In some aspects, the antibody comprises a V_{L} sequence comprising, consisting of, or consisting essentially of SEQ ID NO:178. In some aspects, the antibody comprises a V_{L} sequence comprising, consisting of, or consisting essentially of SEQ ID NO:179.

2.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least about 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.6.2. Excluded V_{L} Sequences

In some embodiments, the V_{L} sequences provided herein do not comprise certain V_{L} sequences. In some aspects, the V_{L} sequence does not comprise, consist of, or consist essentially of a sequence selected from SEQ ID NO:200.

2.7. Pairs

2.7.1. CDR-H3-CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of SEQ ID NOs:80-98, and the CDR-L3 sequence is a CDR-L3 sequence comprising, consisting of, or consisting essentially of SEQ ID NOs: 130-144.

In some aspects, the CDR-H3-CDR-L3 pairs are selected from SEQ ID NO:80 and SEQ ID NO:130; SEQ ID NO:80 and SEQ ID NO:131; SEQ ID NO:80 and SEQ ID NO:132; SEQ ID NO:80 and SEQ ID NO:133; SEQ ID NO:80 and SEQ ID NO:134; SEQ ID NO:80 and SEQ ID NO:135; SEQ ID NO:80 and SEQ ID NO:136; SEQ ID NO:80 and SEQ ID NO:137; SEQ ID NO:80 and SEQ ID NO:138; SEQ ID NO:80 and SEQ ID NO:139; SEQ ID NO:80 and SEQ ID NO:140; SEQ ID NO:80 and SEQ ID NO:141; SEQ ID NO:80 and SEQ ID NO:142; SEQ ID NO:80 and SEQ ID NO:143; and SEQ ID NO:80 and SEQ ID NO:144.

In some aspects, the CDR-H3-CDR-L3 pairs are selected from SEQ ID NO:81 and SEQ ID NO:130; SEQ ID NO:81 and SEQ ID NO:131; SEQ ID NO:81 and SEQ ID NO:132; SEQ ID NO:81 and SEQ ID NO:133; SEQ ID NO:81 and SEQ ID NO:134; SEQ ID NO:81 and SEQ ID NO:135; SEQ ID NO:81 and SEQ ID NO:136; SEQ ID NO:81 and SEQ ID NO:137; SEQ ID NO:81 and SEQ ID NO:138; SEQ ID NO:81 and SEQ ID NO:139; SEQ ID NO:81 and SEQ ID NO:140; SEQ ID NO:81 and SEQ ID NO:141; SEQ ID NO:81 and SEQ ID NO:142; SEQ ID NO:81 and SEQ ID NO:143; and SEQ ID NO:81 and SEQ ID NO:144.

In some aspects, the CDR-H3-CDR-L3 pairs are selected from SEQ ID NO:82 and SEQ ID NO:130; SEQ ID NO:82 and SEQ ID NO:131; SEQ ID NO:82 and SEQ ID NO:132; SEQ ID NO:82 and SEQ ID NO:133; SEQ ID NO:82 and SEQ ID NO:134; SEQ ID NO:82 and SEQ ID NO:135; SEQ ID NO:82 and SEQ ID NO:136; SEQ ID NO:82 and SEQ ID NO:137; SEQ ID NO:82 and SEQ ID NO:138; SEQ ID NO:82 and SEQ ID NO:139; SEQ ID NO:82 and SEQ ID NO:140; SEQ ID NO:82 and SEQ ID NO:141; SEQ ID NO:82 and SEQ ID NO:142; SEQ ID NO:82 and SEQ ID NO:143; and SEQ ID NO:82 and SEQ ID NO:144.

In some aspects, the CDR-H3-CDR-L3 pairs are selected from SEQ ID NO:83 and SEQ ID NO:130; SEQ ID NO:83 and SEQ ID NO:131; SEQ ID NO:83 and SEQ ID NO:132; SEQ ID NO:83 and SEQ ID NO:133; SEQ ID NO:83 and SEQ ID NO:134; SEQ ID NO:83 and SEQ ID NO:135; SEQ ID NO:83 and SEQ ID NO:136; SEQ ID NO:83 and SEQ ID NO:137; SEQ ID NO:83 and SEQ ID NO:138; SEQ ID NO:83 and SEQ ID NO:139; SEQ ID NO:83 and SEQ ID NO:140; SEQ ID NO:83 and SEQ ID NO:141; SEQ ID NO:83 and SEQ ID NO:142; SEQ ID NO:83 and SEQ ID NO:143; and SEQ ID NO:83 and SEQ ID NO:144.

In some aspects, the CDR-H3-CDR-L3 pairs are selected from SEQ ID NO:84 and SEQ ID NO:130; SEQ ID NO:84 and SEQ ID NO:131; SEQ ID NO:84 and SEQ ID NO:132; SEQ ID NO:84 and SEQ ID NO:133; SEQ ID NO:84 and SEQ ID NO:134; SEQ ID NO:84 and SEQ ID NO:135; SEQ ID NO:84 and SEQ ID NO:136; SEQ ID NO:84 and SEQ ID NO:137; SEQ ID NO:84 and SEQ ID NO:138; SEQ ID NO:84 and SEQ ID NO:139; SEQ ID NO:84 and SEQ ID NO:140; SEQ ID NO:84 and SEQ ID NO:141; SEQ ID NO:84 and SEQ ID NO:142; SEQ ID NO:84 and SEQ ID NO:143; and SEQ ID NO:84 and SEQ ID NO:144.

In some aspects, the CDR-H3-CDR-L3 pairs are selected from SEQ ID NO:85 and SEQ ID NO:130; SEQ ID NO:85 and SEQ ID NO:131; SEQ ID NO:85 and SEQ ID NO:132; SEQ ID NO:85 and SEQ ID NO:133; SEQ ID NO:85 and SEQ ID NO:134; SEQ ID NO:85 and SEQ ID NO:135; SEQ ID NO:85 and SEQ ID NO:136; SEQ ID NO:85 and SEQ ID NO:137; SEQ ID NO:85 and SEQ ID NO:138; SEQ ID NO:85 and SEQ ID NO:139; SEQ ID NO:85 and SEQ ID NO:140; SEQ ID NO:85 and SEQ ID NO:141; SEQ ID NO:85 and SEQ ID NO:142; SEQ ID NO:85 and SEQ ID NO:143; and SEQ ID NO:85 and SEQ ID NO:144.

In some aspects, the CDR-H3-CDR-L3 pairs are selected from SEQ ID NO:86 and SEQ ID NO:130; SEQ ID NO:86 and SEQ ID NO:131; SEQ ID NO:86 and SEQ ID NO:132; SEQ ID NO:86 and SEQ ID NO:133; SEQ ID NO:86 and SEQ ID NO:134; SEQ ID NO:86 and SEQ ID NO:135; SEQ ID NO:86 and SEQ ID NO:136; SEQ ID NO:86 and SEQ ID NO:137; SEQ ID NO:86 and SEQ ID NO:138; SEQ ID NO:86 and SEQ ID NO:139; SEQ ID NO:86 and SEQ ID NO:140; SEQ ID NO:86 and SEQ ID NO:141; SEQ ID NO:86 and SEQ ID NO:142; SEQ ID NO:86 and SEQ ID NO:143; and SEQ ID NO:86 and SEQ ID NO:144.

In some aspects, the CDR-H3-CDR-L3 pairs are selected from SEQ ID NO:87 and SEQ ID NO:130; SEQ ID NO:87 and SEQ ID NO:131; SEQ ID NO:87 and SEQ ID NO:132; SEQ ID NO:87 and SEQ ID NO:133; SEQ ID NO:87 and SEQ ID NO:134; SEQ ID NO:87 and SEQ ID NO:135; SEQ ID NO:87 and SEQ ID NO:136; SEQ ID NO:87 and SEQ ID NO:137; SEQ ID NO:87 and SEQ ID NO:138; SEQ ID NO:87 and SEQ ID NO:139; SEQ ID NO:87 and SEQ ID NO:140; SEQ ID NO:87 and SEQ ID NO:141; SEQ ID NO:87 and SEQ ID NO:142; SEQ ID NO:87 and SEQ ID NO:143; and SEQ ID NO:87 and SEQ ID NO:144.

In some aspects, the CDR-H3-CDR-L3 pairs are selected from SEQ ID NO:88 and SEQ ID NO:130; SEQ ID NO:88 and SEQ ID NO:131; SEQ ID NO:88 and SEQ ID NO:132; SEQ ID NO:88 and SEQ ID NO:133; SEQ ID NO:88 and SEQ ID NO:134; SEQ ID NO:88 and SEQ ID NO:135; SEQ ID NO:88 and SEQ ID NO:136; SEQ ID NO:88 and SEQ ID NO:137; SEQ ID NO:88 and SEQ ID NO:138; SEQ ID NO:88 and SEQ ID NO:139; SEQ ID NO:88 and SEQ ID NO:140; SEQ ID NO:88 and SEQ ID NO:141; SEQ ID NO:88 and SEQ ID NO:142; SEQ ID NO:88 and SEQ ID NO:143; and SEQ ID NO:88 and SEQ ID NO:144.

In some aspects, the CDR-H3-CDR-L3 pairs are selected from SEQ ID NO:89 and SEQ ID NO:130; SEQ ID NO:89 and SEQ ID NO:131; SEQ ID NO:89 and SEQ ID NO:132; SEQ ID NO:89 and SEQ ID NO:133; SEQ ID NO:89 and SEQ ID NO:134; SEQ ID NO:89 and SEQ ID NO:135; SEQ ID NO:89 and SEQ ID NO:136; SEQ ID NO:89 and SEQ ID NO:137; SEQ ID NO:89 and SEQ ID NO:138; SEQ ID NO:89 and SEQ ID NO:139; SEQ ID NO:89 and SEQ ID NO:140; SEQ ID NO:89 and SEQ ID NO:141; SEQ ID NO:89 and SEQ ID NO:142; SEQ ID NO:89 and SEQ ID NO:143; and SEQ ID NO:89 and SEQ ID NO:144.

In some aspects, the CDR-H3-CDR-L3 pairs are selected from SEQ ID NO:90 and SEQ ID NO:130; SEQ ID NO:90 and SEQ ID NO:131; SEQ ID NO:90 and SEQ ID NO:132; SEQ ID NO:90 and SEQ ID NO:133; SEQ ID NO:90 and SEQ ID NO:134; SEQ ID NO:90 and SEQ ID NO:135; SEQ ID NO:90 and SEQ ID NO:136; SEQ ID NO:90 and SEQ ID NO:137; SEQ ID NO:90 and SEQ ID NO:138; SEQ ID NO:90 and SEQ ID NO:139; SEQ ID NO:90 and SEQ ID NO:140; SEQ ID NO:90 and SEQ ID NO:141; SEQ ID NO:90 and SEQ ID NO:142; SEQ ID NO:90 and SEQ ID NO:143; and SEQ ID NO:90 and SEQ ID NO:144.

In some aspects, the CDR-H3-CDR-L3 pairs are selected from SEQ ID NO:91 and SEQ ID NO:130; SEQ ID NO:91 and SEQ ID NO:131; SEQ ID NO:91 and SEQ ID NO:132; SEQ ID NO:91 and SEQ ID NO:133; SEQ ID NO:91 and SEQ ID NO:134; SEQ ID NO:91 and SEQ ID NO:135; SEQ ID NO:91 and SEQ ID NO:136; SEQ ID NO:91 and SEQ ID NO:137; SEQ ID NO:91 and SEQ ID NO:138; SEQ ID NO:91 and SEQ ID NO:139; SEQ ID NO:91 and SEQ ID NO:140; SEQ ID NO:91 and SEQ ID NO:141; SEQ ID NO:91 and SEQ ID NO:142; SEQ ID NO:91 and SEQ ID NO:143; and SEQ ID NO:91 and SEQ ID NO:144.

In some aspects, the CDR-H3-CDR-L3 pairs are selected from SEQ ID NO:92 and SEQ ID NO:130; SEQ ID NO:92 and SEQ ID NO:131; SEQ ID NO:92 and SEQ ID NO:132; SEQ ID NO:92 and SEQ ID NO:133; SEQ ID NO:92 and SEQ ID NO:134; SEQ ID NO:92 and SEQ ID NO:135; SEQ ID NO:92 and SEQ ID NO:136; SEQ ID NO:92 and SEQ ID NO:137; SEQ ID NO:92 and SEQ ID NO:138; SEQ ID NO:92 and SEQ ID NO:139; SEQ ID NO:92 and SEQ ID NO:140; SEQ ID NO:92 and SEQ ID NO:141; SEQ ID NO:92 and SEQ ID NO:142; SEQ ID NO:92 and SEQ ID NO:143; and SEQ ID NO:92 and SEQ ID NO:144.

In some aspects, the CDR-H3-CDR-L3 pairs are selected from SEQ ID NO:93 and SEQ ID NO:130; SEQ ID NO:93 and SEQ ID NO:131; SEQ ID NO:93 and SEQ ID NO:132; SEQ ID NO:93 and SEQ ID NO:133; SEQ ID NO:93 and SEQ ID NO:134; SEQ ID NO:93 and SEQ ID NO:135; SEQ ID NO:93 and SEQ ID NO:136; SEQ ID NO:93 and SEQ ID NO:137; SEQ ID NO:93 and SEQ ID NO:138; SEQ ID NO:93 and SEQ ID NO:139; SEQ ID NO:93 and SEQ ID NO:140; SEQ ID NO:93 and SEQ ID NO:141; SEQ ID NO:93 and SEQ ID NO:142; SEQ ID NO:93 and SEQ ID NO:143; and SEQ ID NO:93 and SEQ ID NO:144.

In some aspects, the CDR-H3-CDR-L3 pairs are selected from SEQ ID NO:94 and SEQ ID NO:130; SEQ ID NO:94 and SEQ ID NO:131; SEQ ID NO:94 and SEQ ID NO:132; SEQ ID NO:94 and SEQ ID NO:133; SEQ ID NO:94 and SEQ ID NO:134; SEQ ID NO:94 and SEQ ID NO:135; SEQ ID NO:94 and SEQ ID NO:136; SEQ ID NO:94 and SEQ ID NO:137; SEQ ID NO:94 and SEQ ID NO:138; SEQ ID NO:94 and SEQ ID NO:139; SEQ ID NO:94 and SEQ ID NO:140; SEQ ID NO:94 and SEQ ID NO:141; SEQ ID NO:94 and SEQ ID NO:142; SEQ ID NO:94 and SEQ ID NO:143; and SEQ ID NO:94 and SEQ ID NO:144.

In some aspects, the CDR-H3-CDR-L3 pairs are selected from SEQ ID NO:95 and SEQ ID NO:130; SEQ ID NO:95 and SEQ ID NO:131; SEQ ID NO:95 and SEQ ID NO:132; SEQ ID NO:95 and SEQ ID NO:133; SEQ ID NO:95 and SEQ ID NO:134; SEQ ID NO:95 and SEQ ID NO:135; SEQ ID NO:95 and SEQ ID NO:136; SEQ ID NO:95 and SEQ ID NO:137; SEQ ID NO:95 and SEQ ID NO:138; SEQ ID NO:95 and SEQ ID NO:139; SEQ ID NO:95 and SEQ ID NO:140; SEQ ID NO:95 and SEQ ID NO:141; SEQ ID NO:95 and SEQ ID NO:142; SEQ ID NO:95 and SEQ ID NO:143; and SEQ ID NO:95 and SEQ ID NO:144.

In some aspects, the CDR-H3-CDR-L3 pairs are selected from SEQ ID NO:96 and SEQ ID NO:130; SEQ ID NO:96 and SEQ ID NO:131; SEQ ID NO:96 and SEQ ID NO:132; SEQ ID NO:96 and SEQ ID NO:133; SEQ ID NO:96 and SEQ ID NO:134; SEQ ID NO:96 and SEQ ID NO:135; SEQ ID NO:96 and SEQ ID NO:136; SEQ ID NO:96 and SEQ ID NO:137; SEQ ID NO:96 and SEQ ID NO:138; SEQ ID NO:96 and SEQ ID NO:139; SEQ ID NO:96 and SEQ ID NO:140; SEQ ID NO:96 and SEQ ID NO:141; SEQ ID NO:96 and SEQ ID NO:142; SEQ ID NO:96 and SEQ ID NO:143; and SEQ ID NO:96 and SEQ ID NO:144.

In some aspects, the CDR-H3-CDR-L3 pairs are selected from SEQ ID NO:97 and SEQ ID NO:130; SEQ ID NO:97 and SEQ ID NO:131; SEQ ID NO:97 and SEQ ID NO:132; SEQ ID NO:97 and SEQ ID NO:133; SEQ ID NO:97 and SEQ ID NO:134; SEQ ID NO:97 and SEQ ID NO:135; SEQ ID NO:97 and SEQ ID NO:136; SEQ ID NO:97 and SEQ ID NO:137; SEQ ID NO:97 and SEQ ID NO:138; SEQ ID NO:97 and SEQ ID NO:139; SEQ ID NO:97 and SEQ ID NO:140; SEQ ID NO:97 and SEQ ID NO:141; SEQ ID NO:97 and SEQ ID NO:142; SEQ ID NO:97 and SEQ ID NO:143; and SEQ ID NO:97 and SEQ ID NO:144.

In some aspects, the CDR-H3-CDR-L3 pairs are selected from SEQ ID NO:98 and SEQ ID NO:130; SEQ ID NO:98 and SEQ ID NO:131; SEQ ID NO:98 and SEQ ID NO:132; SEQ ID NO:98 and SEQ ID NO:133; SEQ ID NO:98 and SEQ ID NO:134; SEQ ID NO:98 and SEQ ID NO:135; SEQ ID NO:98 and SEQ ID NO:136; SEQ ID NO:98 and SEQ ID NO:137; SEQ ID NO:98 and SEQ ID NO:138; SEQ ID NO:98 and SEQ ID NO:139; SEQ ID NO:98 and SEQ ID NO:140; SEQ ID NO:98 and SEQ ID NO:141; SEQ ID NO:98 and SEQ ID NO:142; SEQ ID NO:98 and SEQ ID NO:143; and SEQ ID NO:98 and SEQ ID NO:144.

2.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least about 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least about 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.7.1.2. Excluded CDR-H3-CDR-L3 Pairs

In some embodiments, the CDR-H3-CDR-L3 pairs provided herein do not comprise certain CDR-H3-CDR-L3 pairs. In some aspects, the CDR-H3 sequence is not selected from SEQ ID NO:195, and the CDR-L3 sequence is not selected from SEQ ID NO:198. In some aspects, the CDR-H3 sequence is not selected from SEQ ID NO:195, the CDR-L3 sequence is not selected from SEQ ID NO:198, and the CDR-H2 sequence is not selected from SEQ ID NO:193 or 194 (Chothia or Kabat).

2.7.1.3. Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least about 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least about 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

2.7.2. CDR-H2-CDR-L2 Pairs

In some embodiments, the antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of SEQ ID NOs:42-60, and the CDR-L2 sequence is a CDR-L2 sequence comprising, consisting of, or consisting essentially of SEQ ID NOs: 115-129.

In some aspects, the CDR-H1 sequence is a Kabat CDR-H2 sequence comprising, consisting of, or consisting essentially of SEQ ID NOs:61-79, and the CDR-L2 sequence is a CDR-L2 sequence comprising, consisting of, or consisting essentially of SEQ ID NOs: 115-129.

2.7.2.1. Variants of CDR-H2-CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least about 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least about 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

2.7.2.2. Excluded CDR-H2-CDR-L2 Pairs

In some embodiments, the CDR-H2-CDR-L2 pairs provided herein do not comprise certain CDR-H2-CDR-L2 pairs.

In some aspects, the Chothia CDR-H2 sequence is not selected from SEQ ID NO:193, and the CDR-L2 sequence is not selected from SEQ ID NO:197. In some aspects, the Kabat CDR-H2 sequence is not selected from SEQ ID NO:194, and the CDR-L2 sequence is not selected from SEQ ID NO:197.

2.7.3. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of SEQ ID NOs:145-164, and the V_{L} sequence is a V_{L} sequence comprising, consisting of, or consisting essentially of SEQ ID NOs: 165-179.

In some aspects, the V_{H}-V_{L} pairs are selected from SEQ ID NO:145 and SEQ ID NO:165; SEQ ID NO:145 and SEQ ID NO:166; SEQ ID NO:145 and SEQ ID NO:167; SEQ ID NO:145 and SEQ ID NO:168; SEQ ID NO:145 and SEQ ID NO:169; SEQ ID NO:145 and SEQ ID NO:170; SEQ ID NO:145 and SEQ ID NO:171; SEQ ID NO:145 and SEQ ID NO:172; SEQ ID NO:145 and SEQ ID NO:173; SEQ ID NO:145 and SEQ ID NO:174; SEQ ID NO:145 and SEQ ID NO:175; SEQ ID NO:145 and SEQ ID NO:176; SEQ ID NO:145 and SEQ ID NO:177; SEQ ID NO:145 and SEQ ID NO:178, and SEQ ID NO:145 and SEQ ID NO:179.

In some aspects, the V_{H}-V_{L} pairs are selected from SEQ ID NO:146 and SEQ ID NO:165; SEQ ID NO:146 and SEQ ID NO:166; SEQ ID NO:146 and SEQ ID NO:167; SEQ ID NO:146 and SEQ ID NO:168; SEQ ID NO:146 and SEQ ID NO:169; SEQ ID NO:146 and SEQ ID NO:170; SEQ ID NO:146 and SEQ ID NO:171; SEQ ID NO:146 and SEQ ID NO:172; SEQ ID NO:146 and SEQ ID NO:173; SEQ ID NO:146 and SEQ ID NO:174; SEQ ID NO:146 and SEQ ID NO:175; SEQ ID NO:146 and SEQ ID NO:176; SEQ ID NO:146 and SEQ ID NO:177; SEQ ID NO:146 and SEQ ID NO:178, and SEQ ID NO:146 and SEQ ID NO:179.

In some aspects, the V_{H}-V_{L} pairs are selected from SEQ ID NO:147 and SEQ ID NO:165; SEQ ID NO:147 and SEQ ID NO:166; SEQ ID NO:147 and SEQ ID NO:167; SEQ ID NO:147 and SEQ ID NO:168; SEQ ID NO:147 and SEQ ID NO:169; SEQ ID NO:147 and SEQ ID NO:170; SEQ ID NO:147 and SEQ ID NO:171; SEQ ID NO:147 and SEQ ID NO:172; SEQ ID NO:147 and SEQ ID NO:173; SEQ ID NO:147 and SEQ ID NO:174; SEQ ID NO:147 and SEQ ID NO:175; SEQ ID NO:147 and SEQ ID NO:176; SEQ ID NO:147 and SEQ ID NO:177; SEQ ID NO:147 and SEQ ID NO:178, and SEQ ID NO:147 and SEQ ID NO:179.

In some aspects, the V_{H}-V_{L} pairs are selected from SEQ ID NO:148 and SEQ ID NO:165; SEQ ID NO:148 and SEQ ID NO:166; SEQ ID NO:148 and SEQ ID NO:167; SEQ ID NO:148 and SEQ ID NO:168; SEQ ID NO:148 and SEQ ID NO:169; SEQ ID NO:148 and SEQ ID NO:170; SEQ ID NO:148 and SEQ ID NO:171; SEQ ID NO:148 and SEQ ID NO:172; SEQ ID NO:148 and SEQ ID NO:173; SEQ ID NO:148 and SEQ ID NO:174; SEQ ID NO:148 and SEQ ID NO:175; SEQ ID NO:148 and SEQ ID NO:176; SEQ ID NO:148 and SEQ ID NO:177; SEQ ID NO:148 and SEQ ID NO:178, and SEQ ID NO:148 and SEQ ID NO:179.

In some aspects, the V_{H}-V_{L} pairs are selected from SEQ ID NO:149 and SEQ ID NO:165; SEQ ID NO:149 and SEQ ID NO:166; SEQ ID NO:149 and SEQ ID NO:167; SEQ ID NO:149 and SEQ ID NO:168; SEQ ID NO:149 and SEQ ID NO:169; SEQ ID NO:149 and SEQ ID NO:170; SEQ ID NO:149 and SEQ ID NO:171; SEQ ID NO:149 and SEQ ID NO:172; SEQ ID NO:149 and SEQ ID NO:173; SEQ ID NO:149 and SEQ ID NO:174; SEQ ID NO:149 and SEQ ID NO:175; SEQ ID NO:149 and SEQ ID NO:176; SEQ ID NO:149 and SEQ ID NO:177; SEQ ID NO:149 and SEQ ID NO:178, and SEQ ID NO:149 and SEQ ID NO:179.

In some aspects, the V_{H}-V_{L} pairs are selected from SEQ ID NO:150 and SEQ ID NO:165; SEQ ID NO:150 and SEQ ID NO:166; SEQ ID NO:150 and SEQ ID NO:167; SEQ ID NO:150 and SEQ ID NO:168; SEQ ID NO:150 and SEQ ID NO:169; SEQ ID NO:150 and SEQ ID NO:170; SEQ ID NO:150 and SEQ ID NO:171; SEQ ID NO:150 and SEQ ID NO:172; SEQ ID NO:150 and SEQ ID NO:173; SEQ ID NO:150 and SEQ ID NO:174; SEQ ID NO:150 and SEQ ID NO:175; SEQ ID NO:150 and SEQ ID NO:176; SEQ ID NO:150 and SEQ ID NO:177; SEQ ID NO:150 and SEQ ID NO:178, and SEQ ID NO:150 and SEQ ID NO:179.

In some aspects, the V_{H}-V_{L} pairs are selected from SEQ ID NO:151 and SEQ ID NO:165; SEQ ID NO:151 and SEQ ID NO:166; SEQ ID NO:151 and SEQ ID NO:167; SEQ ID NO:151 and SEQ ID NO:168; SEQ ID NO:151 and SEQ ID NO:169; SEQ ID NO:151 and SEQ ID NO:170; SEQ ID NO:151 and SEQ ID NO:171; SEQ ID NO:151 and SEQ ID NO:172; SEQ ID NO:151 and SEQ ID NO:173; SEQ ID NO:151 and SEQ ID NO:174; SEQ ID NO:151 and SEQ ID NO:175; SEQ ID NO:151 and SEQ ID NO:176; SEQ ID NO:151 and SEQ ID NO:177; SEQ ID NO:151 and SEQ ID NO:178, and SEQ ID NO:151 and SEQ ID NO:179.

In some aspects, the V_{H}-V_{L} pairs are selected from SEQ ID NO:152 and SEQ ID NO:165; SEQ ID NO:152 and SEQ ID NO:166; SEQ ID NO:152 and SEQ ID NO:167; SEQ ID NO:152 and SEQ ID NO:168; SEQ ID NO:152 and SEQ ID NO:169; SEQ ID NO:152 and SEQ ID NO:170; SEQ ID NO:152 and SEQ ID NO:171; SEQ ID NO:152 and SEQ ID NO:172; SEQ ID NO:152 and SEQ ID NO:173; SEQ ID NO:152 and SEQ ID NO:174; SEQ ID NO:152 and SEQ ID NO:175; SEQ ID NO:152 and SEQ ID NO:176; SEQ ID NO:152 and SEQ ID NO:177; SEQ ID NO:152 and SEQ ID NO:178, and SEQ ID NO:152 and SEQ ID NO:179.

In some aspects, the V_{H}-V_{L} pairs are selected from SEQ ID NO:153 and SEQ ID NO:165; SEQ ID NO:153 and SEQ ID NO:166; SEQ ID NO:153 and SEQ ID NO:167; SEQ ID NO:153 and SEQ ID NO:168; SEQ ID NO:153 and SEQ ID NO:169; SEQ ID NO:153 and SEQ ID NO:170; SEQ ID NO:153 and SEQ ID NO:171; SEQ ID NO:153 and SEQ ID NO:172; SEQ ID NO:153 and SEQ ID NO:173; SEQ ID NO:153 and SEQ ID NO:174; SEQ ID NO:153 and SEQ ID NO:175; SEQ ID NO:153 and SEQ ID NO:176; SEQ ID NO:153 and SEQ ID NO:177; SEQ ID NO:153 and SEQ ID NO:178, and SEQ ID NO:153 and SEQ ID NO:179.

In some aspects, the V_{H}-V_{L} pairs are selected from SEQ ID NO:154 and SEQ ID NO:165; SEQ ID NO:154 and SEQ ID NO:166; SEQ ID NO:154 and SEQ ID NO:167; SEQ ID NO:154 and SEQ ID NO:168; SEQ ID NO:154 and SEQ ID NO:169; SEQ ID NO:154 and SEQ ID NO:170; SEQ ID NO:154 and SEQ ID NO:171; SEQ ID NO:154 and SEQ ID NO:172; SEQ ID NO:154 and SEQ ID NO:173; SEQ ID NO:154 and SEQ ID NO:174; SEQ ID NO:154 and SEQ ID NO:175; SEQ ID NO:154 and SEQ ID NO:176; SEQ ID NO:154 and SEQ ID NO:177; SEQ ID NO:154 and SEQ ID NO:178, and SEQ ID NO:154 and SEQ ID NO:179.

In some aspects, the V_{H}-V_{L} pairs are selected from SEQ ID NO:155 and SEQ ID NO:165; SEQ ID NO:155 and SEQ ID NO:166; SEQ ID NO:155 and SEQ ID NO:167; SEQ ID NO:155 and SEQ ID NO:168; SEQ ID NO:155 and SEQ ID NO:169; SEQ ID NO:155 and SEQ ID NO:170; SEQ ID NO:155 and SEQ ID NO:171; SEQ ID NO:155 and SEQ ID NO:172; SEQ ID NO:155 and SEQ ID NO:173; SEQ ID NO:155 and SEQ ID NO:174; SEQ ID NO:155 and SEQ ID NO:175; SEQ ID NO:155 and SEQ ID NO:176; SEQ ID NO:155 and SEQ ID NO:177; SEQ ID NO:155 and SEQ ID NO:178, and SEQ ID NO:155 and SEQ ID NO:179.

In some aspects, the V_{H}-V_{L} pairs are selected from SEQ ID NO:156 and SEQ ID NO:165; SEQ ID NO:156 and SEQ ID NO:166; SEQ ID NO:156 and SEQ ID NO:167; SEQ ID NO:156 and SEQ ID NO:168; SEQ ID NO:156 and SEQ ID NO:169; SEQ ID NO:156 and SEQ ID NO:170; SEQ ID NO:156 and SEQ ID NO:171; SEQ ID NO:156 and SEQ ID NO:172; SEQ ID NO:156 and SEQ ID NO:173; SEQ ID NO:156 and SEQ ID NO:174; SEQ ID NO:156 and SEQ ID NO:175; SEQ ID NO:156 and SEQ ID NO:176; SEQ ID NO:156 and SEQ ID NO:177; SEQ ID NO:156 and SEQ ID NO:178, and SEQ ID NO:156 and SEQ ID NO:179.

In some aspects, the V_{H}-V_{L} pairs are selected from SEQ ID NO:157 and SEQ ID NO:165; SEQ ID NO:157 and SEQ ID NO:166; SEQ ID NO:157 and SEQ ID NO:167; SEQ ID NO:157 and SEQ ID NO:168; SEQ ID NO:157 and SEQ ID NO:169; SEQ ID NO:157 and SEQ ID NO:170; SEQ ID NO:157 and SEQ ID NO:171; SEQ ID NO:157 and SEQ ID NO:172; SEQ ID NO:157 and SEQ ID NO:173; SEQ ID NO:157 and SEQ ID NO:174; SEQ ID NO:157 and SEQ ID NO:175; SEQ ID NO:157 and SEQ ID NO:176; SEQ ID NO:157 and SEQ ID NO:177; SEQ ID NO:157 and SEQ ID NO:178, and SEQ ID NO:157 and SEQ ID NO:179.

In some aspects, the V_{H}-V_{L} pairs are selected from SEQ ID NO:158 and SEQ ID NO:165; SEQ ID NO:158 and SEQ ID NO:166; SEQ ID NO:158 and SEQ ID NO:167; SEQ ID NO:158 and SEQ ID NO:168; SEQ ID NO:158 and SEQ ID NO:169; SEQ ID NO:158 and SEQ ID NO:170; SEQ ID NO:158 and SEQ ID NO:171; SEQ ID NO:158 and SEQ ID NO:172; SEQ ID NO:158 and SEQ ID NO:173; SEQ ID NO:158 and SEQ ID NO:174; SEQ ID NO:158 and SEQ ID NO:175; SEQ ID NO:158 and SEQ ID NO:176; SEQ ID NO:158 and SEQ ID NO:177; SEQ ID NO:158 and SEQ ID NO:178, and SEQ ID NO:158 and SEQ ID NO:179.

In some aspects, the V_{H}-V_{L} pairs are selected from SEQ ID NO:159 and SEQ ID NO:165; SEQ ID NO:159 and SEQ ID NO:166; SEQ ID NO:159 and SEQ ID NO:167; SEQ ID NO:159 and SEQ ID NO:168; SEQ ID NO:159 and SEQ ID NO:169; SEQ ID NO:159 and SEQ ID NO:170; SEQ ID NO:159 and SEQ ID NO:171; SEQ ID NO:159 and SEQ ID NO:172; SEQ ID NO:159 and SEQ ID NO:173; SEQ ID NO:159 and SEQ ID NO:174; SEQ ID NO:159 and SEQ ID NO:175; SEQ ID NO:159 and SEQ ID NO:176; SEQ ID NO:159 and SEQ ID NO:177; SEQ ID NO:159 and SEQ ID NO:178, and SEQ ID NO:159 and SEQ ID NO:179.

In some aspects, the V_{H}-V_{L} pairs are selected from SEQ ID NO:160 and SEQ ID NO:165; SEQ ID NO:160 and SEQ ID NO:166; SEQ ID NO:160 and SEQ ID NO:167; SEQ ID NO:160 and SEQ ID NO:168; SEQ ID NO:160 and SEQ ID NO:169; SEQ ID NO:160 and SEQ ID NO:170; SEQ ID NO:160 and SEQ ID NO:171; SEQ ID NO:160 and SEQ ID NO:172; SEQ ID NO:160 and SEQ ID NO:173; SEQ ID NO:160 and SEQ ID NO:174; SEQ ID NO:160 and SEQ ID NO:175; SEQ ID NO:160 and SEQ ID NO:176; SEQ ID NO:160 and SEQ ID NO:177; SEQ ID NO:160 and SEQ ID NO:178, and SEQ ID NO:160 and SEQ ID NO:179.

In some aspects, the V_{H}-V_{L} pairs are selected from SEQ ID NO:161 and SEQ ID NO:165; SEQ ID NO:161 and SEQ ID NO:166; SEQ ID NO:161 and SEQ ID NO:167; SEQ ID NO:161 and SEQ ID NO:168; SEQ ID NO:161 and SEQ ID NO:169; SEQ ID NO:161 and SEQ ID NO:170; SEQ ID NO:161 and SEQ ID NO:171; SEQ ID NO:161 and SEQ ID NO:172; SEQ ID NO:161 and SEQ ID NO:173; SEQ ID NO:161 and SEQ ID NO:174; SEQ ID NO:161 and SEQ ID NO:175; SEQ ID NO:161 and SEQ ID NO:176; SEQ ID NO:161 and SEQ ID NO:177; SEQ ID NO:161 and SEQ ID NO:178, and SEQ ID NO:161 and SEQ ID NO:179.

In some aspects, the V_{H}-V_{L} pairs are selected from SEQ ID NO:162 and SEQ ID NO:165; SEQ ID NO:162 and SEQ ID NO:166; SEQ ID NO:162 and SEQ ID NO:167; SEQ ID NO:162 and SEQ ID NO:168; SEQ ID NO:162 and SEQ ID NO:169; SEQ ID NO:162 and SEQ ID NO:170; SEQ ID NO:162 and SEQ ID NO:171; SEQ ID NO:162 and SEQ ID NO:172; SEQ ID NO:162 and SEQ ID NO:173; SEQ ID NO:162 and SEQ ID NO:174; SEQ ID NO:162 and SEQ ID NO:175; SEQ ID NO:162 and SEQ ID NO:176; SEQ ID NO:162 and SEQ ID NO:177; SEQ ID NO:162 and SEQ ID NO:178, and SEQ ID NO:162 and SEQ ID NO:179.

In some aspects, the V_{H}-V_{L} pairs are selected from SEQ ID NO:163 and SEQ ID NO:165; SEQ ID NO:163 and SEQ ID NO:166; SEQ ID NO:163 and SEQ ID NO:167; SEQ ID NO:163 and SEQ ID NO:168; SEQ ID NO:163 and SEQ ID NO:169; SEQ ID NO:163 and SEQ ID NO:170; SEQ ID NO:163 and SEQ ID NO:171; SEQ ID NO:163 and SEQ ID NO:172; SEQ ID NO:163 and SEQ ID NO:173; SEQ ID NO:163 and SEQ ID NO:174; SEQ ID NO:163 and SEQ ID NO:175; SEQ ID NO:163 and SEQ ID NO:176; SEQ ID NO:163 and SEQ ID NO:177; SEQ ID NO:163 and SEQ ID NO:178, and SEQ ID NO:163 and SEQ ID NO:179.

In some aspects, the V_{H}-V_{L} pairs are selected from SEQ ID NO:164 and SEQ ID NO:165; SEQ ID NO:164 and SEQ ID NO:166; SEQ ID NO:164 and SEQ ID NO:167; SEQ ID NO:164 and SEQ ID NO:168; SEQ ID NO:164 and SEQ ID NO:169; SEQ ID NO:164 and SEQ ID NO:170; SEQ ID NO:164 and SEQ ID NO:171; SEQ ID NO:164 and SEQ ID NO:172; SEQ ID NO:164 and SEQ ID NO:173; SEQ ID NO:164 and SEQ ID NO:174; SEQ ID NO:164 and SEQ ID NO:175; SEQ ID NO:164 and SEQ ID NO:176; SEQ ID NO:164 and SEQ ID NO:177; SEQ ID NO:164 and SEQ ID NO:178, and SEQ ID NO:164 and SEQ ID NO:179.

2.7.3.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least about 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least about 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.7.3.2. Excluded V_{H}-V_{L} Pairs

In some embodiments, the V_{H}-V_{L} pairs provided herein do not comprise certain V_{H}-V_{L} pairs. In some aspects, the V_{H} sequence is not selected from SEQ ID NO:199, and the V_{L} sequence is not selected from SEQ ID NO:200.

2.8. Antibodies Comprising All Six CDRs

In some embodiments, the antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of any of SEQ ID NOs: 4-22; the CDR-H2 sequence is a Chothia CDR-H2 sequence comprising, consisting of, or consisting essentially of any of SEQ ID NOs:42-60; the CDR-H3 sequence is a CDR-H3 sequence comprising, consisting of, or consisting essentially of any of SEQ ID NOs:80-98; the CDR-L1 sequence is a CDR-L1 sequence comprising, consisting of, or consisting essentially of any of SEQ ID NOs:100-114; the CDR-L2 sequence is a CDR-L2 sequence comprising, consisting of, or consisting essentially of any of SEQ ID NOs: 115-129; and the CDR-L3 sequence is a CDR-L3 sequence comprising, consisting of, or consisting essentially of any of SEQ ID NOs:130-144.

In some aspects, the CDR-H1 sequence is a Kabat CDR-H1 sequence comprising, consisting of, or consisting essentially of any of SEQ ID NOs:23-41; the CDR-H2 sequence is a Kabat CDR-H2 sequence comprising, consisting of, or consisting essentially of any of SEQ ID NOs: 61-79; the CDR-H3 sequence is a CDR-H3 sequence comprising, consisting of, or consisting essentially of any of SEQ ID NOs:80-98; the CDR-L1 sequence is a CDR-L1 sequence comprising, consisting of, or consisting essentially of any of SEQ ID NOs:100-114; the CDR-L2 sequence is a CDR-L2 sequence comprising, consisting of, or consisting essentially of any of SEQ ID NOs:115-129; and the CDR-L3 sequence is a CDR-L3 sequence comprising, consisting of, or consisting essentially of any of SEQ ID NOs:130-144.

2.8.1. Variants of Antibodies Comprising all Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least about 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least about 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least about 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least about 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least about 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least about 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.8.2. Excluded Six CDR Combinations

In some embodiments, the CDR-H1, CDR-H2, CDR-H3, CDR-L1, CDR-L2, and CDR-L3 provided herein do not comprise certain CDR-H1, CDR-H2, CDR-H3, CDR-L1, CDR-L2, and/or CDR-L3s sequences.

In some aspects, the Chothia CDR-H1 sequence is not selected from SEQ ID NO:191; the Kabat CDR-H1 sequence is not selected from SEQ ID NO:192; the Chothia CDR-H2 sequence is not selected from SEQ ID NO:193; the Kabat CDR-H2 sequence is not selected from SEQ ID NO:194; the CDR-H3 sequence is not selected from SEQ ID NO:195; the CDR-L1 sequence is not selected from SEQ ID NO:196; the CDR-L2 sequence is not selected from SEQ ID NO:197; and/or the CDR-L3 sequence is not selected from SEQ ID NO:198.

2.9. Consensus Sequences

In some embodiments, provided herein are anti-LAG3 antibodies comprising one or more sequences defined by consensus sequences. Each consensus sequence is based, at least in part, on one or more alignments of two or more useful anti-LAG3 CDR sequences provided in this disclosure. Based on such alignments, a person of skill in the art would recognize that different amino acid residues may useful in certain positions of the CDRs. Accordingly, each consensus sequence encompasses two or more useful anti-LAG3 CDR sequences.

In some embodiments, the antibodies comprise one to six of the consensus CDR sequences provided herein. In some embodiments, the antibodies comprise two to six of the consensus CDR sequences provided herein. In some embodiments, the antibodies comprise three to six of the consensus CDR sequences provided herein. In some embodiments, the antibodies comprise four to six of the consensus CDR sequences provided herein. In some embodiments, the antibodies comprise five to six of the consensus CDR sequences provided herein. In some embodiments, the antibodies comprise six of the consensus CDR sequences provided herein. In some embodiments, the antibodies comprise a V_{L} comprising the CDR-L consensus sequence(s). In some embodiments, the antibodies comprise a V_{H} comprising the CDR-H consensus sequence(s). In some embodiments, the antibodies comprise a V_{H} comprising the CDR-H consensus sequence(s) and a V_{L} comprising the CDR-L consensus sequence(s).

2.9.1. CDR-H3 Consensus Sequences

In some embodiments, the antibody comprises a CDR-H3 sequence defined by the consensus sequence α₁-α₂-α₃-α₄-α₅-α₆-α₇-α₈-α₉-α₁₀-α₁₁-D-α₁₃, where α₁ is absent, E, or V; α₂ is absent I, S, W, E, Y, D, or F; α₃ is absent, F, L, I, E A, A, or N; α₄ is absent, G, V, P, or D; α₅ is absent, A, S, E, V, or G; α₆ is F, S, N, or V; α₇ is absent Y, W, or R; α₈ is W, L, D, P, or S; α₉ is N, Y, A, D, or F; α₁₀ is P, A, G, S, or M; α₁₁ is absent, F, L, M, or V; and α₁₃ is Y or V. In certain embodiments, each of α₁, α₂, α₃, α₄, α₅, α₆, and α₇ is absent. In certain embodiments, none of α₁, α₂, α₃, α₄, α₅, α₆, and α₇ is absent. In certain embodiments, only α₅ of α₁, α₂, α₃, α₄, α₅, α₆, and α₇ is absent. In certain embodiments, only α₅ is absent. In certain embodiments, only α₁₁ is absent. In certain embodiments, when α₂ is W, α₄ is V, α₅ is A, α₆ is S, and α₁₀ is G, then α₁₁ is F, L, or V. In certain embodiments, α₂ is E or D, α₄ is P, α₅ is E, α₆ is N, α₁₀ is A or G, and α₁₁ is F, L, or V. In certain embodiments, the antibody comprises a CDR-H3 sequence defined by the consensus sequence E-α₂-α₃-α₄-α₅-α₆-W-D-α₉-α₁₀-α₁₁-D-V where α₂ is S, W, or E; α₃ is A or E; α₄ is V, P, or D; α₅ is A, S, E, or V; α₆ is S or N; α₉ is Y or A; α₁₀ is A or G; and α₁₁ is L or M wherein when α₂ is W, α₄ is V, α₅ is A, α₆ is S, and α₁₀ is G, then α₁₁ is L.

In some embodiments, the antibody comprises a CDR-H3 sequence defined by the consensus sequence V-ß₂-ß₃-G-G-V-R-P-ß₉-S-ß₁₁-D-Y, where ß₂ is F, Y, or D; ß₃ is E or N; ß₉ is Y or F; and ß₁₁ is absent.

2.9.2. Chothia CDR-H1 Consensus Sequences

In some embodiments, the antibody comprises a Chothia CDR-H1 sequence defined by the consensus sequence G-F-γ₃-γ₄-γ₅-γ₆-γ₇, where γ₃ is N or T; γ₄ is I or F; γ₅ is K, N, A, S, R, P, or T; γ₆ is D, S, or E; and γ₇ is T, N, Y, F, S, or L.

In some embodiments, the antibody comprises a Chothia CDR-H1 sequence defined by the consensus sequence G-F-T-F-δ₅-δ₆-δ₇, where δ₅ is 5, R, P, T, or N; δ₆ is S, D, or E; and δ₇ is F, S, or Y.

2.9.3. Chothia CDR-H2 Consensus Sequences

In some embodiments, the antibody comprises a Chothia CDR-H2 sequence defined by the consensus sequence ε₁-ε₂-ε₃-ε₄-ε₅-ε₆, where ε₁ is D, W, or T; ε₂ is P, Y, D, G, or S; ε₃ is Y, D, N, W, or, E; ε₄ is D, A, G, S, T, or N; ε₅ is G or S; and ε₆ is A, D, F, Y, V, N, T, or S. In certain embodiments, ε₁ is W; ε₂ is Y; ε₃ is D; ε₄ is A or G; ε₅ is S; and ε₆ is Y, N, or V. In certain embodiments, ε₁ is T or S; ε₂ is D or S; ε₃ is N or D; ε₄ is S or T; ε₅ is G; and 86 is N, T, or S.

2.9.4. Kabat CDR-H1 Consensus Sequences

In some embodiments, the antibody comprises a Kabat CDR-H1 sequence defined by the consensus sequence ζ₁-ζ₂-ζ₃-ζ₄-ζ₅, where ζ₁ is D, S, or E; ζ₂ is T, N, Y, F, S, or L; ζ₃ is Y, F, G, S, or T; ζ₄ is I or M; and ζ₅ is H or S.

In some embodiments, the antibody comprises a Kabat CDR-H1 sequence defined by the consensus sequence S-η₂-G-M-H, where η₂ is Y or F. In some embodiments, the antibody comprises a Kabat CDR-H1 sequence defined by the consensus sequence η₁-S-η₃-M-H, where η₁ is D, E, or S; and η₃ is S or T.

2.9.5. Kabat CDR-H2 Consensus Sequences

In some embodiments, the antibody comprises a Kabat CDR-H2 sequence defined by the consensus sequence θ₁-θ₂-θ₃-θ₄-θ₅-θ₆-θ₇-θ₈-θ₉-θ₁₀-Y-A-θ₁₃-θ₁₄-θ₁₅-θ₁₆-G, where θ₁ is I, A, V, R, or W; θ₃ is D, W, T, or S; θ₄ is P, Y, D, G, or S; θ₅ is Y, D, N, W, or E; θ₆ is D, A, G, S, T, or N; θ₇ is G or S; θ₈ is A, D, F, Y, N, V, T, or S; θ₉ is T or K; θ₁₀ is D, A, Y or E; θ₁₃ is D, or P; θ₁₄ is S or K; θ₁₅ is V or F; and θ₁₆ is K or Q. In some embodiments, the antibody comprises a Kabat CDR-H2 sequence defined by the consensus sequence θ₁-I-θ₃-Y-D-G-S-θ₈-K-Y-Y-A-D-S-V-K-G, where θ₁ is V or A; θ₃ is W or T; and θ₈ is Y, N, or V. In some embodiments, the antibody comprises a Kabat CDR-H2 sequence defined by the consensus sequence θ₁-I-θ₃-θ₄-θ₅-θ₆-G-θ₈-T-D-Y-A-D-S-V-K-G, where θ₁ is F or V; θ₃ is T or S; θ₄ is S, D, or G; θ₅ is D or N; θ₆ is S or T; and θ₈ is T, S, or N.

2.9.6. CDR-L3 Consensus Sequences

In some embodiments, the antibody comprises a CDR-L3 sequence defined by the consensus sequence Q-Q-ι₃-ι₄-ι₅-ι₆-P-ι₈-ι₉, where ι₃ is Y or D; ι₄ is G, D, S, M, or T; ι₅ is R, S, A, or L; ι₆ is S, T, A, or G; ι₈ is F, L or P; and ι₉ is S, T, or K. In certain embodiments, when ι₅ is S, then ι₅ is S.

In some embodiments, the antibody comprises a CDR-L3 sequence defined by the consensus sequence ι₁-ι₂-ι₃-ι₄-ι₅-ι₆-P-Q-T where ι₁ is S or W; ι₂ is H, T, or Q; ι₃ is G or Y; ι₄ is N, I, or S; and ι₅ is V or F.

2.9.7. CDR-L2 Consensus Sequences

In some embodiments, the antibody comprises a CDR-L2 sequence selected from the group consisting of GASSRAT (SEQ ID NO:115) and LVSKLDS (SEQ ID NO:125).

2.9.8. CDR-L1 Consensus Sequences

In some embodiments, the antibody comprises a CDR-L1 sequence defined by the consensus sequence R-A-S-Q-μ₅-μ₆-μ₇-μ₈-S-V-S-S-μ₁₃-μ₁₄-μ₁₅-A, where μ₅ is absent; μ₁₄ is absent; μ₇ is absent; μ₈ is absent; μ₁₃ is S, N, or G; μ₁₄ is Y, P or N; and μ₁₅ is L or P. In some embodiments, the antibody comprises a CDR-L1 sequence defined by the consensus sequence KSSQSLLDSDGKTYLN (SEQ ID NO:110).

### 3. Germline

In some embodiments, the antibody that specifically binds LAG3 is an antibody comprising a variable region that is encoded by a particular germline gene, or a variant thereof. The illustrative antibodies provided herein comprise variable regions that are encoded by the heavy chain variable region germline genes VH3-23 and VH5-51, or variants thereof and the light chain variable region germline genes Vκ3-20 and Vκ4-1, or variants thereof.

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H} 3 or V_{H} 5 families, or a variant thereof. In some embodiments, a CDR-L sequence provided herein may be combined with a variable region encoded by a variable region germline gene selected from the Vκ₃ or Vκ4 families, or a variant thereof.

### 4. Affinity

In some embodiments, the affinity of the antibody for LAG3 as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸M and 10⁻¹¹M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻¹⁰ M and 10⁻¹¹M.

In some embodiments, the affinity of the antibody for human LAG3, as determined by surface plasmon resonance at 25° C., and as indicated by K_{D}, is between about 1.3×10⁻⁸ M and about 1.93×10⁻¹⁰ M. In some embodiments, the affinity of the antibody for human LAG3 is about 8.63×10⁻⁷ M, about 4.33×10⁻⁸ M, about 3.90×10⁻⁸ M, about 3.10×10⁻⁸M, about 2.40×10⁻⁸ M, about 2.13×10⁻⁸ M, about 1.89×10⁻⁸ M, about 1.52×10⁻⁸ M, about 1.47×10⁻⁸M, about 1.35×10⁻⁸ M, about 1.30×10⁻⁸ M, about 1.03×10⁻⁸ M, about 3.10×10⁻⁹M, about 2.46×10⁻⁹M, about 2.27×10⁻⁹M, about 1.36×10⁻⁹M, about 6.76×10⁻¹° M, about 6.40×10⁻¹° M, or about 4.12×10⁻¹¹ M.

In some embodiments, the affinity of the antibody for human LAG3 expressed on the surface of a cell, as indicated by K_{D}, is between about 78.0 and about 0.19 nM. In some embodiments, the affinity of the antibody for human LAG3 expressed on the surface of a cell is about 78.0 nM, about 40.6 nM, about 39.4 nM, about 35.0 nM, about 3.37 nM, about 1.92 nM, about 1.54 nM, about 1.06 nM, about 0.97 nM, about 0.74 nM, about 0.50 nM, about 0.40 nM, about 0.32 nM, about 0.30 nM, and about 0.19 nM. In some embodiments, the cell is a CHO cell. In some embodiments, the cell is a 293T cell.

In some embodiments, the affinity of the antibody for cynomolgus LAG3, as determined by surface plasmon resonance at 25° C., and as indicated by K_{D}, is between about 4.5×10⁻⁹ M and about 0.3×10⁻⁹ M. In some embodiments, the affinity of the antibody for cynomolgus LAG3 is about 4.5×10⁻⁹ M, about 1.6×10⁻⁹ M, about 1.0×10⁻⁹ M, about 0.7×10⁻⁹M, or about 0.3×10⁻⁹ M.

In some embodiments, the antibody is characterized by a ratio of affinity for human LAG3 to affinity for cynomolgus LAG3, each as determined by surface plasmon resonance at 25° C., and as indicated by K_{D}. In some embodiments, the ratio is from about 0.25 to about 4.5. In some embodiments, the ratio is about 0.25, about 0.5, about 0.7, about 1.0, or about 4.5.

In some embodiments, the affinity of the antibody for cynomolgus LAG3 expressed on the surface of a cell, as indicated by K_{D}, is between about 4.5 and about 0.3 nM. In some embodiments, the affinity of the antibody for cynomolgus LAG3 expressed on the surface of a cell is about 4.5 nM, about 1.6 nM, about 1.0 nM, about 0.7 nM, or about 0.3 nM. In some embodiments, the cell is a CHO cell.

In some embodiments the antibody has a kₐ of at least about 10⁴ M⁻¹×sec⁻¹. In some embodiments the antibody has a kₐ of at least about 10⁵ M⁻¹×sec⁻¹. In some embodiments the antibody has a kₐ of at least about 10⁶ M⁻¹×sec⁻¹. In some embodiments the antibody has a kₐ of at least about 10⁷ M⁻¹×sec⁻¹. In some embodiments the antibody has a kₐ of between about 10⁴ M⁻¹×sec⁻¹ and about 10⁸ M⁻¹×sec⁻¹. In some embodiments the antibody has a kₐ of between about 10⁵ M⁻¹×sec⁻¹ and about 10⁸ M⁻¹×sec⁻¹.

In some embodiments the antibody has a kₐ when associating with human LAG3, as determined by surface plasmon resonance at 25° C., of between about 5.02×10⁴ M⁻¹×sec⁻¹ and about 5.31×10⁷ M⁻¹×sec⁻¹. In some embodiments the antibody has a kₐ when associating with human LAG3 of about 2.67×10³ M⁻¹×sec⁻¹, about 5.02×10⁴ M⁻¹×sec⁻¹, about 1.61×10⁵ M⁻¹×sec⁻¹, about 2.61×10⁵ M⁻¹×sec⁻¹, about 3.12×10⁵ M⁻¹×sec⁻¹, about 4.35×10⁵ M⁻¹×sec⁻¹, about 4.60×10⁵ M⁻¹×sec⁻¹, about 4.72×10⁵ M⁻¹×sec⁻¹, about 5.60×10⁵ M⁻¹×sec⁻¹, about 7.90×10⁵ M⁻¹×sec⁻¹, about 7.94×10⁵ M⁻¹×sec⁻¹, about 1.06×10⁶ M⁻¹×sec⁻¹, about 1.24×10⁶ M⁻¹×sec⁻¹, about 1.29×10⁶ M⁻¹×sec⁻¹, about 1.31×10⁶ M⁻¹×sec⁻¹, about 1.64×10⁶ M⁻¹×sec⁻¹, about 1.65×10⁶ M⁻¹×sec⁻¹, about 1.12×10⁷ M⁻¹×sec⁻¹, or about 5.35×10⁷ M⁻¹×sec⁻¹.

In some embodiments the antibody has a k_{d} of about 10⁻⁵ sec⁻¹ or less. In some embodiments the antibody has a k_{d} of about 10⁻⁴ sec⁻¹ or less. In some embodiments the antibody has a k_{d} of about 10⁻³ sec⁻¹ or less. In some embodiments the antibody has a k_{d} of between about 10⁻² sec⁻¹ and about 10⁻⁶ sec⁻¹. In some embodiments the antibody has a k_{d} of between about 10⁻² sec⁻¹ and about 10⁻⁵ sec⁻¹. In some embodiments the antibody has a k_{d} of between about 10⁻² sec⁻¹ and about 10⁻⁴ sec⁻¹. In some embodiments the antibody has a k_{d} of between about 10⁻³ sec⁻¹ and about 10⁻⁵ sec⁻¹.

In some embodiments the antibody has a k_{d} when dissociating from human LAG3, as determined by surface plasmon resonance at 25° C., of between about 2.79×10⁻² sec⁻¹ and about 6.78×10⁻⁵ sec⁻¹. In some embodiments the antibody has a k_{d} when dissociating from human LAG3 of about 1.22×10⁻¹ sec⁻¹, about 7.10×10⁻² sec⁻¹, about 2.79×10⁻² sec⁻¹, about 2.75×10⁻² sec⁻¹, about 2.34×10⁻² sec⁻¹, about 1.96×10⁻² sec⁻¹, about 1.70×10⁻² sec⁻¹, about 1.52×10⁻² sec⁻¹, about 1.10×10⁻² sec⁻¹, about 9.90×10⁻³ sec⁻¹, about 6.20×10⁻³ sec⁻¹, about 4.22×10⁻³ sec⁻¹, about 2.30×10⁻³ sec⁻¹, about 8.07×10⁻⁴ sec⁻¹, about 6.27×10⁻⁴ sec⁻¹, about 5.36×10⁻⁴ sec⁻¹, about 5.15×10⁻⁴ sec⁻¹, about 3.02×10⁻⁴ sec⁻¹, or about 6.78×10⁻⁵ sec⁻¹.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and k_{d} are determined according to the methods described in the Examples provided herein.

### 5. Epitope Bins

In some embodiments, the antibody binds the same epitope as the scFvFc antibody provided in SEQ ID NO:145. In some embodiments, the antibody binds to a different epitope from the scFvFc antibody provided in SEQ ID NO:145. In some embodiments, the antibody binds to part of the epitope bound by the scFvFc antibody provided in SEQ ID NO:145. In some embodiments, the antibody competes for epitope binding with the scFvFc antibody provided in SEQ ID NO:145. In some embodiments, the antibody does not compete for epitope binding with the scFvFc antibody provided in SEQ ID NO:145.

### 6. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 7. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

An alteration in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγR1, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. USA., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 8. Preparation of Antibodies

8.1. Antigen Preparation

The LAG3 antigen to be used for isolation of the antibodies may be intact LAG3 or a fragment of LAG3. The intact LAG3, or fragment of LAG3, may be in the form of an isolated protein or protein expressed by a cell. Other forms of LAG3 useful for generating antibodies will be apparent to those skilled in the art.

8.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, Calif., incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

8.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. USA., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

8.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 9. Vectors, Host Cells, and Recombinant Methods

The invention also provides isolated nucleic acids encoding anti-LAG3 antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-LAG3 antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-LAG3 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 10. Pharmaceutical Compositions and Methods of Administration

Any of the antibodies provided herein can be provided in any appropriate pharmaceutical composition and be administered by any suitable route of administration. Suitable routes of administration include, but are not limited to, the inhalation, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a cosolvent. Illustrative examples of cosolvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

10.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

10.2. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody provided herein, based on weight of the antibody, administered to prevent, treat, manage, or ameliorate a disorder, or one or more symptoms thereof in a subject is about 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is about 0.1 mg to 200 mg, about 0.1 mg to 100 mg, about 0.1 mg to 50 mg, about 0.1 mg to 25 mg, about 0.1 mg to 20 mg, about 0.1 mg to 15 mg, about 0.1 mg to 10 mg, about 0.1 mg to 7.5 mg, about 0.1 mg to 5 mg, about 0.1 to 2.5 mg, about 0.25 mg to 20 mg, about 0.25 to 15 mg, about 0.25 to 12 mg, about 0.25 to 10 mg, about 0.25 mg to 7.5 mg, about 0.25 mg to 5 mg, about 0.25 mg to 2.5 mg, about 0.5 mg to 20 mg, about 0.5 to 15 mg, about 0.5 to 12 mg, about 0.5 to 10 mg, about 0.5 mg to 7.5 mg, about 0.5 mg to 5 mg, about 0.5 mg to 2.5 mg, about 1 mg to 20 mg, about 1 mg to 15 mg, about 1 mg to 12 mg, about 1 mg to 10 mg, about 1 mg to 7.5 mg, about 1 mg to 5 mg, or about 1 mg to 2.5 mg.

The dose can be administered according to a suitable schedule, for example, once, two times, three times, or for times weekly. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least about 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least about 1 day, 2 days, 3 days, 5 days, 10 days, 15 days, 30 days, 45 days, 2 months, 75 days, 3 months, or 6 months.

### 11. Therapeutic Applications

For therapeutic applications, the antibodies of the invention are administered to a mammal, generally a human, in a pharmaceutically acceptable dosage form such as those known in the art and those discussed above. For example, the antibodies of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

The antibodies provided herein may be useful for the treatment of any disease or condition involving LAG3. In some embodiments, the disease or condition is a disease or condition that can be diagnosed by overexpression of LAG3. In some embodiments, the disease or condition is a disease or condition that can benefit from treatment with an anti-LAG3 antibody. In some embodiments, the disease or condition is a cancer.

Any suitable cancer may be treated with the antibodies provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In particular embodiments, the cancer is a cancer of epithelial origin. In some aspects, the cancer is a carcinoma. In some aspects, the cancer is selected from an adenocarcinoma, a squamous cell carcinoma, an adenosquamos carcinoma, an anaplastic carcinoma, a large cell carcinoma, small cell carcinoma, and carcinoma of unknown primary origin.

### 12. Diagnostic Applications

In some embodiments, the antibodies provided herein are used in diagnostic applications. For example, an ant-LAG3 antibody may be useful in assays for LAG3 protein. In some aspects the antibody can be used to detect the expression of LAG3 in various cells and tissues. These assays may be useful, for example, in making a diagnosis and/or prognosis for a disease, such as a cancer.

In some diagnostic and prognostic applications, the antibody may be labeled with a detectable moiety. Suitable detectable moieties include, but are not limited to radioisotopes, fluorescent labels, and enzyme-substrate labels. In another embodiment, the anti-LAG3 antibody need not be labeled, and the presence of the antibody can be detected using a labeled antibody which specifically binds to the anti-LAG3 antibody.

### 13. Affinity Purification Reagents

The antibodies of the invention may be used as affinity purification agents. In this process, the antibodies may be immobilized on a solid phase such a resin or filter paper, using methods well known in the art. The immobilized antibody is contacted with a sample containing the LAG3 protein (or fragment thereof) to be purified, and thereafter the support is washed with a suitable solvent that will remove substantially all the material in the sample except the LAG3 protein, which is bound to the immobilized antibody. Finally, the support is washed with another suitable solvent, such as glycine buffer, pH 5.0, that will release the LAG3 protein from the antibody.

### 14. Kits

In some embodiments, an anti-LAG3 antibody provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-LAG3 antibody. In some embodiments, the anti-LAG3 antibody is provided in the form of a pharmaceutical composition.

### EXAMPLES

### Example 1: Generation of Mouse and Humanized Antibodies

Balb/C mice were immunized with the extracellular domain of human LAG3 fused with human Fc (R&D Systems) using standard immunization methods. The spleens and/or lymph nodes of the mice were harvested and fused with P3X cells to generate hybridomas (Aragen Biosciences, Morgan Hill, Calif.), similar to what has been previously described (Chronopoulou et al., 2014, Methods Mol Biol. 1131:47-70; Kim, et al., 2014, Methods Mol Biol. 1131:33-45; each incorporated by reference in its entirety).

Total RNA was extracted from hybridoma cells using QIAGEN RNeasy Mini Kit (Cat No. 74104) and converted to cDNA using a Clontech SMARTer RACE cDNA Amplification Kit (Cat. No. 634923; Lake Pharma, Belmont, Calif.). Positive clones were identified by gel electrophoresis, cloned using an Invitrogen TOPO kit, and sequenced using standard Sanger methods. Mouse single-chain antibodies were constructed by using total gene synthesis using optimized E. Coli codons and cloned into a standard cell-free expression vector (Yin et al., 2012, mAbs 4:217-225, incorporated by reference in its entirety). Murine IgG 421.61.4.5G11 (5G11) was selected.

The CDRs for 5G11 were grafted onto human antibody frameworks VH1-69 and Vk2-30 by standard methodology (Kuramochi et al., 2014, Methods Mol. Biol. 1060:123-137, incorporated by reference in its entirety) to yield humanized antibody h5G11-2.

### Example 2: Generation and Primary Screening of Anti-LAG3 Antibodies

Antibody Fab and scFv libraries were constructed using a standard overlap extension PCR protocol with mutagenic primers targeting complementary determining regions (CDRs). See Heckman and Pease, Nat. Protoc., 2007, 2:924-932, incorporated by reference in its entirety. Selections for novel antibodies were performed using standard ribosome display protocols. See Dreier and Plückthun, Methods Mol. Biol., 2003, 687:283-306, Clifton, N.J., incorporated by reference in its entirety. Specifically, scFv and Fab ribosome display selections were performed according to published protocols. See Hanes and Plückthun, Proc. Natl. Acad. Sci. U.S.A., 1997, 94:4937-4942; Stafford et al., 2014, Protein Eng. Des. Sel. 27:97-109; each incorporated by reference in its entirety. After multiple rounds of selection, the DNA from RT-PCR output was cloned into an optimized vector for cell-free expression using standard molecular biology techniques. See Yin et al., mAbs, 2012, 4:217-225, incorporated by reference in its entirety. All constructs were HIS- and FLAG-tagged to streamline purification and testing during screening.

Libraries of antibody variants generated by selection workflow were transformed into E. coli and grown on agar plates with antibiotic (kanamycin). Individual colonies were grown in liquid broth (TB+kanamycin), and used as a template for DNA amplification via rolling circle amplification (RCA). The variants were then expressed in cell-free protein synthesis reactions as described in Zawada et al., 2011, Biotechnol. Bioeng. 108:1570-1578, incorporated by reference in its entirety.

Briefly, cell-free extracts were treated with 50 μM iodoacetamide for 30 min at room temperature (20° C.) and added to a premix containing cell-free components (see Groff et al., mAbs, 2014, 6:671-678, incorporated by reference in its entirety) and 10% (v/v) RCA DNA template (approximately 10 μg/mL DNA) for variants of interest. For Fab selection, 2.5 μg/mL trastuzumab LC DNA was also added to the reactions. Sixty microliters of cell-free reactions were incubated at 30° C. for 12 hr on a shaker at 650 rpm in 96-well plates. Four hundred to one-thousand-five-hundred colonies were screened, depending on the predicted diversity of different selection campaigns.

Following synthesis, each reaction was diluted 1:50 into PBST (PBS at pH 7.4 with 0.2% Tween-20+0.2% BSA) and expressed variants were tested for functional activity via ELISA-based binding to recombinant human LAG3 extracellular domain (ECD) (Acro Biosystems; R&D Systems). Standard ELISA-based methods were employed. Specifically, 384-well plates were coated with 2 μg/mL recombinant LAG3 diluted in bicarbonate buffer, and then blocked with BSA. Antibody variants of interest were allowed to bind to the LAG3-coated plates, and detected with secondary antibodies (e.g., HRP-conjugated anti-human Fc or anti-FLAG) and then detected with chemiluminescent substrate (Pierce ELISA SuperSignal™ Substrate). Chemiluminescence was quantified on a Molecular Devices SpectraMax® M5 plate reader. Top hits were selected based on ELISA signal or signal/noise ratio and their associated DNA constructs were sequenced. Based on functional activity and sequence analysis, a subset of variants was selected for further scale-up and characterization.

### Example 3: Secondary Screening of Antibodies

The top leads from the initial round of screening were cultured and plasmid minipreps were performed using a QIAprep® 96 Turbo miniprep kit (Qiagen) according to the manufacturer's instructions. 10 μg/mL miniprepped DNA was added to 4 mL cell-free reactions and incubated overnight for 12 hr at 30° C., at 650 rpm. For Fab selection, 2.5 ug/mL trastuzumab LC DNA was also added.

Expressed variants from clarified cell-free reactions were purified via immobilized metal ion affinity chromatography (IMAC) purification using a semi-automated high throughput batch purification method. Briefly, purifications were performed in a 96-well plate format where 50 μL/well of IMAC resin (Ni Sepharose High Performance, GE Healthcare) was equilibrated in IMAC binding buffer (50 mM Tris pH 8.0, 300 mM NaCl, 10 mM imidazole), incubated with 1 mL cell-free reaction for 15 minutes followed by two washes in IMAC binding buffer. His-tagged antibody variants were then eluted using 200 μL IMAC elution buffer (50 mM Tris pH 8.0, 300 mM NaCl, 500 mM imidazole) and buffer exchanged into PBS using a 96-well Zeba plate (7 kD MWCO, Thermo Fisher). Purified antibodies were quantified via high throughput capillary electrophoresis using the LabChip GXII (Perkin Elmer) against a Herceptin standard curve, according to the manufacturer's instructions.

### Example 4: Antibody Selection and Maturation

Primary and secondary screening with humanized antibody h5G11-2 yielded antibodies designated SRP1627 in the Examples below. Ribosome display was used to affinity mature antibody 26H10 (SEQ ID NOS:199 & 200) yielding antibodies designated SRP1449 or 1449 in the Examples below. Antibody SRP1448-D09 was identified by screening a naive scFv antibody library against LAG3. Affinity maturation of SRP1448-D09 using ribosome display yielded antibodies designated SRP1558 in the Examples below. Anti-LAG-3 Fabs were identified by selecting from a Fab TRIM library against recombinant LAG3 protein using ribosome display (Stafford et al., Protein Eng Des Sel 2014, 27:97-109, incorporated by reference in its entirety). Primary and secondary screening yielded LAG-3 Fab antibodies designated SRP1496 in the Examples below. Affinity maturation of antibody SRP1496-A04 using ribosome display yielded antibodies designated SRP1648 in the Examples below. All ribosome display selections were screened by cloning the output into a cell free expression vector for small-scale expression followed by characterization by ELISA, biacore, cell binding, and ligand competition.

The mouse antibody 421.61.4.5G11 was constructed from the VH and VL variable domains in the table below and mouse constant domains. The human and humanized antibodies were constructed from the VH and VL variable domains in the table below and human constant domains. Additional human antibodies are constructed in either scFvFc or IgG format. The scFvFc format contains a VH domain, followed by a linker domain (for instance, a GGGGSGGGGSGGGGS SEQ ID NO:188 linker or a APGPSAPSHRSLPSRAFG SEQ ID NO:189 linker from Tang et al., 1996, J. Biol. Chem. 271:15682-15686, incorporated by reference in its entirety), then the VL domain, and then the human scFvFc constant domains. The mouse and human antibody sequences start with an N-terminal methionine to enable expression in cell-free. Additional variable domains can also be expressed in a mammalian system by fusing an N-terminal signal peptide instead of an N-terminal methionine. Additional antibodies can also be expressed with or without a C-terminal affinity tags (e.g. His or FlagHis, SEQ ID NO:190).

TABLE 5
Antibody Sequences

| | VH | | VL | |
|---|---|---|---|---|
| Antibody Name | Name | SEQ ID NO | Name | SEQ ID NO |
| 421.61.4.5G11 | 5G11-VH | 146 | 5G11-VL | 165 |
| SRP1627-A02 | SRP1627-A02-VH | 147 | SRP1627-A02-VL | 166 |
| SRP1627-A11 | SRP1627-A11-VH | 148 | SRP1627-A11-VL | 167 |
| SRP1627-B01 | SRP1627-B01-VH | 149 | SRP1627-B01-VL | 168 |
| h5G11-2 | h5G11-2-VH | 150 | h5G11-2-VL | 169 |
| SRP1449-B03 | SRP1449-B03-VH | 151 | SRP1449-B03-VL | 170 |
| SRP1449-B07 | SRP1449-B07-VH | 152 | SRP1449-B07-VL | 171 |
| SRP1449-D05 | SRP1449-D05-VH | 153 | SRP1449-D05-VL | 172 |
| SRP1449-F01 | SRP1449-F01-VH | 154 | SRP1449-F01-VL | 173 |
| SRP1449-G09.2 | SRP1449-G09.2-VH | 155 | SRP1449-G09.2-VL | 174 |
| SRP1558-A06 | SRP1558-A06-VH | 156 | SRP1558-A06-VL | 175 |
| SRP1558-E11 | SRP1558-E11-VH | 157 | SRP1558-E11-VL | 176 |
| SRP1558-F01 | SRP1558-F01-VH | 158 | SRP1558-F01-VL | 177 |
| SRP1448-D09 | SRP1448-D09-VH | 159 | SRP1448-D09-VL | 178 |
| SRP1496-A03 | SRP1496-A03-VH | 160 | trastuzumab-VL | 179 |
| SRP1496-A04 | SRP1496-A04-VH | 161 | trastuzumab-VL | 179 |
| SRP1496-B08 | SRP1496-B08-VH | 162 | trastuzumab-VL | 179 |
| SRP1648-B07 | SRP1648-B07-VH | 163 | trastuzumab-VL | 179 |
| SRP1648-E02 | SRP1648-E02-VH | 164 | trastuzumab-VL | 179 |

### Example 5: Affinity and Kinetic Binding Analyses

Anti-Flag M2 IgG (Sigma-Aldrich # F9291) was immobilized onto a CMS chip (GE Life Sciences) using amine coupling chemistry (from Amine Coupling Kit, GE Life Sciences). The immobilization steps were carried out at a flow rate of 25 μL/min in 1×HBS-EP+ buffer (GE Life Sciences; 10× Stock diluted before use). The sensor surfaces were activated for 7 min with a mixture of NHS (0.05 M) and EDC (0.2 M). The Anti-Flag M2 IgG was injected over all 4 flow cells at a concentration of 25 μg/mL in 10 mM sodium acetate, pH 4.5, for 7 min. Ethanolamine (1 M, pH 8.5) was injected for 7 min to block any remaining activated groups. An average of 12,000 response units (RU) of capture antibody was immobilized on each flow cell.

Off-rate and Kinetic binding experiments were performed at 25° C. using 1×HBS-EP+ buffer. Test and control antibodies were injected over the Anti-Flag surface at concentrations of 5-10 μg/mL for 12 seconds at a flow rate of 10 μL/min on flow cells 2, 3 and 4, followed by a buffer wash for 30 seconds at the same flow rate. Kinetic characterization of antibody samples was carried out with a single concentration of antigen (for off-rate ranking) or a dilution series of antigen (for kinetic characterization) and 1 injection of 0 nM antigen. After capturing ligand (antibody) on the anti-Flag surface, the analyte (human LAG3-Fc, R&D Systems #2319-L3; or cynomolgus LAG3-Fc, accession #NC_022282.1) was bound at 50, 25, 12.5, 6.25, and 0 nM for 180 seconds, followed by a 600 second dissociation phase at a flow rate of 50 μl/min. Between each ligand capture and analyte binding cycle, regeneration was carried out using 2 injections of 10 mM glycine pH 2.0 for 30 seconds at 30 μL/min, followed by a 30 second buffer wash step.

The data were fit with the Biacore T200 Evaluation software, using a 1:1 Langmuir binding model. K_{D} (affinity, nM) was determined as a ratio of the kinetic rate constants calculated from the fits of the association and dissociation phases.

### Example 6: ELISA Binding

Standard ELISA methods were used to compare binding to human and cynomolgus recombinant LAG-3. Specifically, 384-well plates were coated with 2 μg/mL recombinant LAG3 (human LAG3-Fc or cynomolgus LAG3-Fc) diluted in bicarbonate buffer, and then blocked with BSA. A dilution series of antibody variants were allowed to bind to the LAG3-coated plates, and detected with secondary antibodies (e.g., HRP-conjugated anti-human Fab or anti-FLAG) and then detected with chemiluminescent substrate (Pierce ELISA SuperSignal Substrate). Chemiluminescence was quantified on a Molecular Devices SpectraMax® M5 plate reader. ELISA EC50s were calculated.

### Example 7: Cell Binding

Antibody variants were tested in a fluorescence-activated cell sorting (FACS) cell-binding assay. Chinese Hamster Ovary (CHO) cells or HEK293T cells stably expressing the human target molecule LAG3 on the cell surface (CHO-LAG3, 293T-LAG3) were used to screen for cell binders by flow cytometry. Parental CHO or 293T cells were used as a negative control to determine background-binding levels. Cells were cultured in RPMI with 10% FCS Penicillin/Streptomycin (or Pen/Strep) and glutamine (or Gln) and split every 3-4 days at 10⁵ cells/ml.

A mix of parental CHO cells and CHO-LAG3 cells (or 293T and 293T-LAG3 cells) was prepared as follows: Parental CHO cells were washed 2× in PBS then incubated in PBS containing 1 nM CellTrace™ Oregon Green488® (Life Technologies) at 37° C. for 30 minutes. Cells were then washed 2× with RPMI w/10% fetal calf serum (or FCS), washed 2× with FACS buffer (PBS w/2% FCS), suspended thoroughly in ice-cold FACS buffer at a final concentration of 2×106 cells/ml and kept on ice. CHO-LAG3 cells were similarly washed with FACS buffer and kept on ice at 2×106 cells/ml. Parental CHO cells and CHO-LAG3 cells were then mixed to obtain a 1:1 cell suspension and seeded at 100 μl per well on 96 well polypropylene plates. Plates were spun at 1500 rpm for 5 minutes and cell pellets were suspended in 50 μl FACS buffer containing 6-12 point dilutions of anti-LAG3 variants starting from concentrations of ˜100-200 nM antibody, dispensed using BioMekFX (Beckman Coulter). Cells were then incubated on ice for 1 hr, washed with FACS buffer and incubated for 1 hr on ice with 50 μl FACS buffer containing 2.5 μg/ml R-Phycoerythrin-conjugated Goat Anti-Human IgG (Jackson ImmunoResearch) or AF647-conjugated Goat Anti-mouse IgG (Life Technologies) dispensed using BioMekFX (Beckman Coulter). Cells were then washed 2× with FACS buffer and fixed for 10 minutes in 200 μl PBS with 2% PFA prior to fluorescence detection. Samples were acquired using a Beckton Dickinson LSRII FACS. Mean Fluorescence Intensity of LAG3 antibody binding was analyzed using Tree Star, Inc. FlowJo® software.

### Example 8: Cell-based MHCII Competition

Top variants that showed cell-binding activity were tested in a fluorescence-activated cell sorting (FACS) cell-based competition assay. DAUDI cells express high levels of Major Histocompatibility Class II (MHCII) molecules, a natural ligand for LAG3, on the cell surface. DAUDI cells were used to screen for antibodies that inhibit binding of HIS-tagged (ACRO) or biotinylated recombinant human LAG3 protein (rhLAG3) to MHCII expressed on the cell surface.

DAUDI cells were cultured in RPMI w/10% FCS Pen/Strep and Gln and split every 3-4 days at 105 cells/ml. Cells were washed 2× with FACS buffer (PBS w/2% FCS), thoroughly in ice-cold FACS buffer at a final concentration of 1×10⁶ cells/ml and seeded at 100 μl per well on 96 well polypropylene plates. Plates were spun at 1500 rpm for 5 minutes and cell pellets were suspended in 50 μl FACS buffer containing 8 point 1:3 dilutions (2× concentrated) of anti-LAG3 antibody variants, starting from high concentration of ˜600 nM. 50 μl FACS buffer containing 10-20 μg/ml of the HIS-tagged rhLAG3 protein or 40 μg/ml of the biotinylated rhLAG3 protein were then added to the cells. Cell were then incubated in ice for 1 hr, washed with FACS buffer and incubated for 1 hr in ice with 50 μl FACS buffer containing 2 μg/ml R-Phycoerythrin-conjugated Streptavidin (eBiosciences) or 1 μg/ml R-Phycoerythrin-conjugated anti-HIS IgG (Abcam). Cell were washed 2× with FACS buffer and fixed for 10 minutes in 200 μl PBS w/2% PFA prior to acquisition.

### Example 9: Effect of Anti-PD-1 in Combination with Anti-LAG3 Antibodies on IFN-γ Production in a CMV Recall Assay and Dendritic Cell (DC)/CD-4+ T Cell Mixed Lymphocyte Reaction (MLR)

CMV Recall Assay

CD14⁺ monocytes and CD3⁺ T cells were obtained from peripheral blood mononuclear (PBMC) isolated from CMV⁺ human donors (AllCells, Alameda, Calif.) using MACS Cell Separation kits (Miltenyi Biotec). CD14⁺ monocytes were differentiated into immature dendritic cells (DC) by culturing cells at 1e6 cells/ml for 7 days in presence of GM-CSF and IL-4 (Peprotech) in X-Vivo 15 media (Lonza) containing 2% human AB serum (Sigma-Aldrich), penicillin-streptomycin (Corning Mediatech) and GlutaMAX (Life Technologies). Following differentiation, DCs were matured by culturing in X-Vivo 15+2% human AB serum media at 1e6 cells/ml for 2 days in the presence of GM-CSF, IL-4, TNF-a, IL-1b, IL-6 (Peprotech) and prostaglandin E2 (Sigma-Aldrich). To set-up the CMV recall assay, mature DCs were collected, washed and 10,000 DCs and 100,000 pan CD3⁺ T cells were plated per well in a 96-well U-bottom plate in a total volume of 100 ul media containing peptide pools for the CMV IE-1 and CMV pp65 protein (Miltenyi Biotec). Anti-PD-1 and/or anti-LAG-3 IgG antibodies (50 ul) were added starting at a final concentration of 133-400 nM with 5-fold serial dilutions. Cells were co-cultured with peptides and antibodies for 5-6 days. Conditioned media was collected and tested for human IFN-g levels by ELISA (BD Biosciences).

DC/CD4⁺ T cell mixed lymphocyte reaction (MLR)

Allogeneic CD14⁺ monocytes and CD4⁺ T cells were obtained from PBMC isolated from human donors using MACS Cell Separation kits. CD14⁺ monocytes were differentiated into immature DC by culturing cells at 1e6 cells/ml cell density for 7 days in presence of GM-CSF and IL-4 in RPMI media containing 10% fetal bovine serum, penicillin-streptomycin and GlutaMAX. Following differentiation, DCs were matured by culturing in RPMI+10% FBS media at 1e6 cells/ml cell density for 2 days in the presence of GM-CSF, IL-4, TNF-a, IL-1b, IL-6 and prostaglandin E2. To set-up the DC/CD4⁺ T cell MLR, mature DCs were collected, washed and 10,000 DCs and 100,000 CD4⁺ T cells were plated per well in a 96-well U-bottom plate in a total volume of 100 ul media. Anti-PD-1 and/or anti-LAG-3 IgG antibodies (50 ul, final volume of 150 ul per well) were added starting at a final concentration of 133-400 nM with 5-fold serial dilutions. Cells were co-cultured with peptides and antibodies for 5-6 days. Conditioned media was collected and tested for human IFN-g levels by ELISA.

### Example 10: Characteristics of Illustrative Anti-LAG3 Antibodies

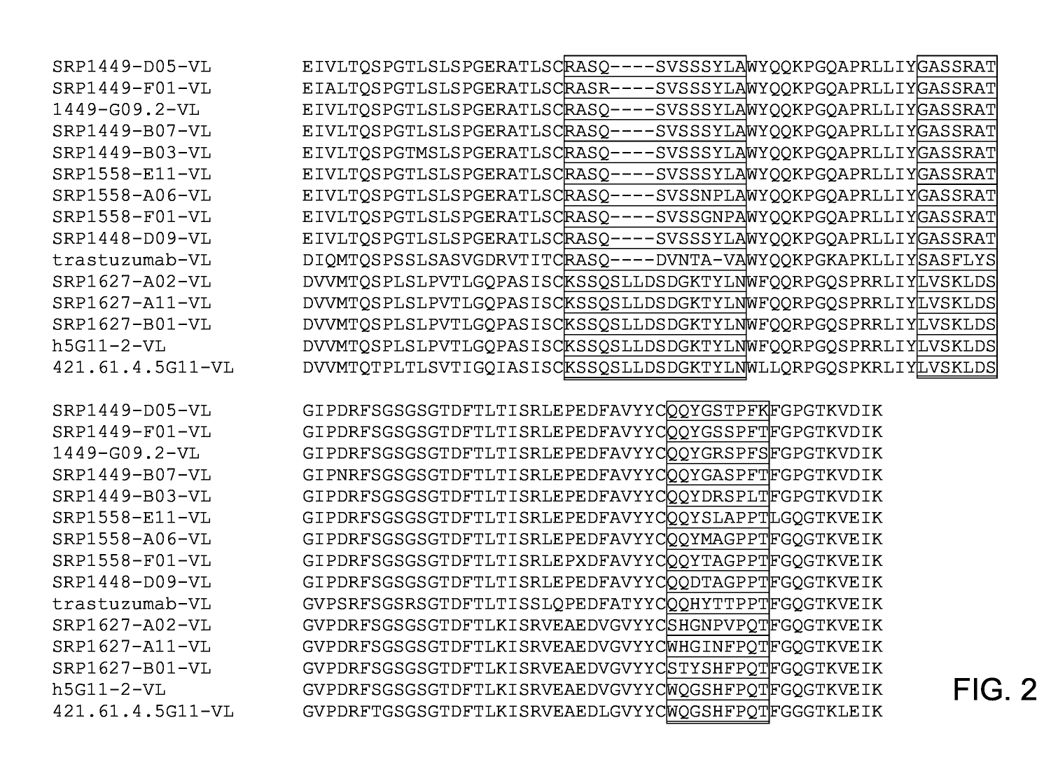

FIG. 1 provides an alignment of the V_{H} sequences provided herein. FIG. 2 provides an alignment of the V_{L} sequences provided herein. Chothia CDR sequences are highlighted, and Kabat CDR sequences are underlined.

Tables 6 and 7 provide results obtained using the illustrative antibodies described herein. Table 6 presents the results of binding assays for antibodies provided herein. Table 7 provides the results of functional assays provided herein.

TABLE 6
Binding Assays

| | | | | | | | | Human | Human | Cyno |
|---|---|---|---|---|---|---|---|---|---|---|
| | | | Human LAG3 | | | Cyno LAG3 | | LAG3 | LAG3 | LAG3 |
| Antibody | | | (Biacore) | | | (Biacore) | | (CHO) | (293T) | (293T) |
| | | SEQ ID | kₐ | k_{d} | K_{D} | k_{d} | K_{D} | K_{D} | K_{D} | K_{D} |
| Name | Scaffold | NO(s) | (1/Ms) | (1/s) | (M) | (1/s) | (M) | (nM) | (nM) | (nM) |
| 421.61.4.5G11 | Murine | 146 | 5.02E+04 | 5.15E−04 | 1.03E−08 | 6.41E−04 | 4.51E−09 | 18.0 | | 4.5 |
| | IgG | 165 | | | | | | | | |
| SRP1627-A02 | ScFvFc | | 1.64E+06 | 7.10E−02 | 4.33E−08 | | | | 0.97 | +++ |
| SRP1627-A11 | ScFvFc | | 3.12E+05 | 4.22E−03 | 1.35E−08 | | | | 0.74 | +++ |
| SRP1627-B01 | ScFvFc | | 1.04E+06 | 1.52E−02 | 1.47E−08 | | | | 0.19 | nd |
| h5G11-2 | ScFvFc | | 2.67E+03 | 2.30E−03 | 8.63E−07 | | | | 78 | nd |
| SRP1449-B03 | ScFvFc | | 4.72E+05 | 3.02E−04 | 6.4E−10 | | | 1.92 | | |
| SRP1449-B07 | ScFvFc | | 7.94E+05 | 5.36E−04 | 6.76E−10 | | | 1.06 | | |
| SRP1449-D05 | ScFvFc | | 4.60E+05 | 6.27E−04 | 1.36E−09 | | | 1.54 | | |
| SRP1449-F01 | ScFvFc | | 2.61E+05 | 8.07E−04 | 3.1E−09 | | | 3.37 | | |
| 1449-G09.2 | ScFvFc | | 1.65E+06 | 6.78E−05 | 4.12E−11 | | | 0.32 | | |
| SRP1558-A06 | ScFvFc | | 7.9E+05 | 9.9E−03 | 1.3E−08 | | | | 0.3 | 0.3 |
| SRP1558-E11 | ScFvFc | | 5.6E+05 | 1.7E−02 | 3.1E−08 | | | | 0.5 | 1.0 |
| SRP1558-F01 | ScFvFc | | 4.35E+05 | 1.1E−02 | 2.4E−08 | | | | 0.5 | 0.7 |
| SRP1448-D09 | ScFvFc | | 1.61E+05 | 6.2E−03 | 3.9E−08 | | | | 0.4 | 1.6 |
| SRP1496-A03 | IgG | 160 | 1.24E+06 | 2.34E−02 | 1.89E−08 | | | | 40.6 | |
| | | 179 | | | | | | | | |
| SRP1496-A04 | IgG | 161 | 1.29E+06 | 1.96E−02 | 1.52E−08 | | | | 35.0 | |
| | | 179 | | | | | | | | |
| SRP1496-B08 | IgG | 162 | 1.31E+06 | 2.79E−02 | 2.13E−08 | | | | 39.4 | |
| | | 179 | | | | | | | | |
| SRP1648-B07 | IgG | 163 | 1.12E+07 | 2.75E−02 | 2.46E−09 | | | | positive | |
| | | 179 | | | | | | | | |
| SRP1648-E02 | IgG | 164 | 5.35E+07 | 1.22E−01 | 2.27E−09 | | | | positive | |
| | | 179 | | | | | | | | |

nd = not detected
+++ = binding observed,
K_{D} not calculated

TABLE 7
Functional Assays

| | MHCII | | Cyno ELISA |
|---|---|---|---|
| | Blockade | Functional | Reactivity |
| Antibody | IC₅₀ (nM) | Activity | EC₅₀ (nM) |
| 421.61.4.5G11 | 64.3 | positive | |
| SRP1627-A02 | 1.7 | | |
| SRP1627-A11 | 1.6 | | |
| SRP1627-B01 | 0.4 | | |
| h5G11-2 | nd | | |
| SRP1449-B03 | 1.0 | Not tested | |
| SRP1449-B07 | 1.2 | Not tested | |
| SRP1449-D05 | 2.3 | Not tested | |
| SRP1449-F01 | 1.7 | Not tested | |
| 1449-G09.2 | 1.1 | positive | |
| SRP1558-A06 | Not tested | positive | |
| SRP1558-E11 | Not tested | Not tested | |
| SRP1558-F01 | Not tested | Not tested | |
| SRP1448-D09 | 1.9 | Not tested | |
| SRP1496-A03 | 41.3 | | 4.2 |
| SRP1496-A04 | 55.8 | | 1.6 |
| SRP1496-B08 | 28.6 | | 2.6 |
| SRP1648-B07 | 4.5 | | |
| SRP1648-E02 | 4.1 | | |

### Example 11: Sequences

Table 8 provides sequences referred to herein. In Table 8, the numbering scheme is indicated as Chothia or Kabat for the sequences where the scheme is significant, e.g., for CDR-H1 and CDR-H2 regions. Otherwise, the scheme is not indicated, and those of skill will recognize that either numbering scheme, or another, can apply.

TABLE 8
Sequences.

| SEQ ID | | | | | |
|---|---|---|---|---|---|
| NO: | Molecule | Region | Scheme | Sequence | Length |
| 1 | Human LAG3 | | | MWEAQFLGLLFLQPLWVAPVKPLQPG | 525 |
| | | | | AEVPVVWAQEGAPAQLPCSPTIPLQD | |
| | | | | LSLLRRAGVTWQHQPDSGPPAAAPGH | |
| | | | | PLAPGPHPAAPSSWGPRPRRYTVLSV | |
| | | | | GPGGLRSGRLPLQPRVQLDERGRQRG | |
| | | | | DFSLWLRPARRADAGEYRAAVHLRDR | |
| | | | | ALSCRLRLRLGQASMTASPPGSLRAS | |
| | | | | DWVILNCSFSRPDRPASVHWFRNRGQ | |
| | | | | GRVPVRESPHHHLAESFLFLPQVSPM | |
| | | | | DSGPWGCILTYRDGFNVSIMYNLTVL | |
| | | | | GLEPPTPLTVYAGAGSRVGLPCRLPA | |
| | | | | GVGTRSFLTAKWTPPGGGPDLLVTGD | |
| | | | | NGDFTLRLEDVSQAQAGTYTCHIHLQ | |
| | | | | EQQLNATVTLAIITVTPKSFGSPGSL | |
| | | | | GKLLCEVTPVSGQERFVWSSLDTPSQ | |
| | | | | RSFSGPWLEAQEAQLLSQPWQCQLYQ | |
| | | | | GERLLGAAVYFTELSSPGAQRSGRAP | |
| | | | | GALPAGHLLLFLILGVLSLLLLVTGA | |
| | | | | FGFHLWRRQWRPRRFSALEQGIHPPQ | |
| | | | | AQSKIEELEQEPEPEPEPEPEPEPEP | |
| | | | | EPEQL | |
| 2 | Macaca LAG3 | | | MWEAQFLGLLFLQPLWVAPVKPPQPG | 533 |
| | | | | AEISVVWAQEGAPAQLPCSPTIPLQD | |
| | | | | LSLLRRAGVTWQHQPDSGPPAXAPGH | |
| | | | | PPVPGHRPAAPYSWGPRPRRYTVLSV | |
| | | | | GPGGLRSGRLPLQPRVQLDERGRQRG | |
| | | | | DFSLWLRPARRADAGEYRATVHLRDR | |
| | | | | ALSCRLRLRVGQASMTASPPGSLRTS | |
| | | | | DWVILNCSFSRPDRPASVHWFRSRGQ | |
| | | | | GRVPVQGSPHHHLAESFLFLPHVGPM | |
| | | | | DSGLWGCILTYRDGFNVSIMYNLTVL | |
| | | | | GLEPATPLTVYAGAGSRVELPCRLPP | |
| | | | | AVGTQSFLTAKWAPPGGGPDLLVAGD | |
| | | | | NGDFTLRLEDVSQAQAGTYICHIRLQ | |
| | | | | GQQLNATVTLAIITVTPKSFGSPGSL | |
| | | | | GKLLCEVTPASGQEHFVWSPLNTPSQ | |
| | | | | RSFSGPWLEAQEAQLLSQPWQCQLHQ | |
| | | | | GERLLGAAVYFTELSSPGAQRSGRAP | |
| | | | | GALRAGHLPLFLILGVLFLLLLVTGA | |
| | | | | FGFHLWRRQWRPRRFSALEQGIHPPQ | |
| | | | | AQSKIEELEQEPELEPEPELERELGP | |
| | | | | EPEPGPEPEPEQL | |
| 3 | Mouse LAG3 | | | MREDLLLGFLLLGLLWEAPVVSSGPG | 521 |
| | | | | KELPVVWAQEGAPVHLPCSLKSPNLD | |
| | | | | PNFLRRGGVIWQHQPDSGQPTPIPAL | |
| | | | | DLHQGMPSPRQPAPGRYTVLSVAPGG | |
| | | | | LRSGRQPLHPHVQLEERGLQRGDFSL | |
| | | | | WLRPALRTDAGEYHATVRLPNRALSC | |
| | | | | SLRLRVGQASMIASPSGVLKLSDWVL | |
| | | | | LNCSFSRPDRPVSVHWFQGQNRVPVY | |
| | | | | NSPRHFLAETFLLLPQVSPLDSGTWG | |
| | | | | CVLTYRDGFNVSITYNLKVLGLEPVA | |
| | | | | PLTVYAAEGSRVELPCHLPPGVGTPS | |
| | | | | LLIAKWTPPGGGPELPVAGKSGNFTL | |
| | | | | HLEAVGLAQAGTYTCSIHLQGQQLNA | |
| | | | | TVTLAVITVTPKSFGLPGSRGKLLCE | |
| | | | | VTPASGKERFVWRPLNNLSRSCPGPV | |
| | | | | LEIQEARLLAERWQCQLYEGQRLLGA | |
| | | | | TVYAAESSSGAHSARRISGDLKGGHL | |
| | | | | VLVLILGALSLFLLVAGAFGFHWWRK | |
| | | | | QLLLRRFSALEHGIQPFPAQRKIEEL | |
| | | | | ERELETEMGQEPEPEPEPQLEPEPRQ | |
| | | | | L | |
| 4 | SRP1496- | CDR-H1 | Chothia | GFNINDT | 7 |
| | A03-VH | | | | |
| 5 | SRP1496- | CDR-H1 | Chothia | GFNINDT | 7 |
| | A04-VH | | | | |
| 6 | SRP1496- | CDR-H1 | Chothia | GFNINDT | 7 |
| | B08-VH | | | | |
| 7 | SRP1648- | CDR-H1 | Chothia | GFNIADT | 7 |
| | B07-VH | | | | |
| 8 | SRP1648-E02- | CDR-H1 | Chothia | GFNINDN | 7 |
| | VH | | | | |
| 9 | SRP1449- | CDR-H1 | Chothia | GFTFSSY | 7 |
| | B03-VH | | | | |
| 10 | SRP1449-F01- | CDR-H1 | Chothia | GFTFSSY | 7 |
| | VH | | | | |
| 11 | SRP1449- | CDR-H1 | Chothia | GFTFSSY | 7 |
| | B07-VH | | | | |
| 12 | 1449-G09.2- | CDR-H1 | Chothia | GFTFSSY | 7 |
| | VH | | | | |
| 13 | SRP1449- | CDR-H1 | Chothia | GFTFRSF | 7 |
| | D05-VH | | | | |
| 14 | SRP1558-F01- | CDR-H1 | Chothia | GFTFPDS | 7 |
| | VH | | | | |
| 15 | SRP1448- | CDR-H1 | Chothia | GFTFTDS | 7 |
| | D09-VH | | | | |
| 16 | SRP1558- | CDR-H1 | Chothia | GFTFSES | 7 |
| | A06-VH | | | | |
| 17 | SRP1558-E11- | CDR-H1 | Chothia | GFTFTSS | 7 |
| | VH | | | | |
| 18 | SRP1627- | CDR-H1 | Chothia | GFNINDY | 7 |
| | A02-VH | | | | |
| 19 | SRP1627- | CDR-H1 | Chothia | GFNINDY | 7 |
| | A11-VH | | | | |
| 20 | h5G11-2-VH | CDR-H1 | Chothia | GFNIKDY | 7 |
| 21 | SRP1627- | CDR-H1 | Chothia | GFNITDL | 7 |
| | B01-VH | | | | |
| 22 | 421.61.4. | CDR-H1 | Chothia | GFNIKDY | 7 |
| | 5G11-VH | | | | |
| 23 | SRP1496- | CDR-H1 | Kabat | DTYIH | 5 |
| | A03-VH | | | | |
| 24 | SRP1496- | CDR-H1 | Kabat | DTYIH | 5 |
| | A04-VH | | | | |
| 25 | SRP1496- | CDR-H1 | Kabat | DTYIH | 5 |
| | B08-VH | | | | |
| 26 | SRP1648- | CDR-H1 | Kabat | DTFIH | 5 |
| | B07-VH | | | | |
| 27 | SRP1648-E02- | CDR-H1 | Kabat | DNYIH | 5 |
| | VH | | | | |
| 28 | SRP1449- | CDR-H1 | Kabat | SYGMH | 5 |
| | B03-VH | | | | |
| 29 | SRP1449-F01- | CDR-H1 | Kabat | SYGMH | 5 |
| | VH | | | | |
| 30 | SRP1449- | CDR-H1 | Kabat | SYGMH | 5 |
| | B07-VH | | | | |
| 31 | 1449-G09.2- | CDR-H1 | Kabat | SYGMH | 5 |
| | VH | | | | |
| 32 | SRP1449- | CDR-H1 | Kabat | SFGMH | 5 |
| | D05-VH | | | | |
| 33 | SRP1558-F01- | CDR-H1 | Kabat | DSSMS | 5 |
| | VH | | | | |
| 34 | SRP1448- | CDR-H1 | Kabat | DSSMS | 5 |
| | D09-VH | | | | |
| 35 | SRP1558- | CDR-H1 | Kabat | ESTMS | 5 |
| | A06-VH | | | | |
| 36 | SRP1558-E11- | CDR-H1 | Kabat | SSSMS | 5 |
| | VH | | | | |
| 37 | SRP1627- | CDR-H1 | Kabat | DYFMH | 5 |
| | A02-VH | | | | |
| 38 | SRP1627- | CDR-H1 | Kabat | DYFMH | 5 |
| | A11-VH | | | | |
| 39 | h5G11-2-VH | CDR-H1 | Kabat | DYYMH | 5 |
| 40 | SRP1627- | CDR-H1 | Kabat | DLYMH | 5 |
| | B01-VH | | | | |
| 41 | 421.61.4. | CDR-H1 | Kabat | DYYMH | 5 |
| | 5G11-VH | | | | |
| 42 | SRP1496- | CDR-H2 | Chothia | DPYDGA | 6 |
| | A03-VH | | | | |
| 43 | SRP1496- | CDR-H2 | Chothia | DPYDGA | 6 |
| | A04-VH | | | | |
| 44 | SRP1496- | CDR-H2 | Chothia | DPYDGA | 6 |
| | B08-VH | | | | |
| 45 | SRP1648- | CDR-H2 | Chothia | DPYDGD | 6 |
| | B07-VH | | | | |
| 46 | SRP1648-E02- | CDR-H2 | Chothia | DPYDGF | 6 |
| | VH | | | | |
| 47 | SRP1449- | CDR-H2 | Chothia | WYDASY | 6 |
| | B03-VH | | | | |
| 48 | SRP1449-F01- | CDR-H2 | Chothia | WYDGSY | 6 |
| | VH | | | | |
| 49 | SRP1449- | CDR-H2 | Chothia | WYDGSN | 6 |
| | B07-VH | | | | |
| 50 | 1449-G09.2- | CDR-H2 | Chothia | WYDGSY | 6 |
| | VH | | | | |
| 51 | SRP1449- | CDR-H2 | Chothia | WYDGSV | 6 |
| | D05-VH | | | | |
| 52 | SRP1558-F01- | CDR-H2 | Chothia | TDNSGN | 6 |
| | VH | | | | |
| 53 | SRP1448- | CDR-H2 | Chothia | TGNSGT | 6 |
| | D09-VH | | | | |
| 54 | SRP1558- | CDR-H2 | Chothia | TSDSGT | 6 |
| | A06-VH | | | | |
| 55 | SRP1558-E11- | CDR-H2 | Chothia | SDDTGS | 6 |
| | VH | | | | |
| 56 | SRP1627- | CDR-H2 | Chothia | DPWNGD | 6 |
| | A02-VH | | | | |
| 57 | SRP1627- | CDR-H2 | Chothia | DPWNGD | 6 |
| | A11-VH | | | | |
| 58 | h5G11-2-VH | CDR-H2 | Chothia | DPENGD | 6 |
| 59 | SRP1627- | CDR-H2 | Chothia | DPWNGD | 6 |
| | B01-VH | | | | |
| 60 | 421.61.4. | CDR-H2 | Chothia | DPENGD | 6 |
| | 5G11-VH | | | | |
| 61 | SRP1496- | CDR-H2 | Kabat | IIDPYDGATDYADSVKG | 17 |
| | A03-VH | | | | |
| 62 | SRP1496- | CDR-H2 | Kabat | IIDPYDGATDYADSVKG | 17 |
| | A04-VH | | | | |
| 63 | SRP1496- | CDR-H2 | Kabat | IIDPYDGATDYADSVKG | 17 |
| | B08-VH | | | | |
| 64 | SRP1648- | CDR-H2 | Kabat | IIDPYDGDTDYADSVKG | 17 |
| | B07-VH | | | | |
| 65 | SRP1648-E02- | CDR-H2 | Kabat | IIDPYDGFTAYADSVKG | 17 |
| | VH | | | | |
| 66 | SRP1449- | CDR-H2 | Kabat | AIWYDASYKYYADSVKG | 17 |
| | B03-VH | | | | |
| 67 | SRP1449-F01- | CDR-H2 | Kabat | VIWYDGSYKYYADSVKG | 17 |
| | VH | | | | |
| 68 | SRP1449- | CDR-H2 | Kabat | VIWYDGSNKYYADSVKG | 17 |
| | B07-VH | | | | |
| 69 | 1449-G09.2- | CDR-H2 | Kabat | VIWYDGSYKYYADSVKG | 17 |
| | VH | | | | |
| 70 | SRP1449- | CDR-H2 | Kabat | VIWYDGSVKYYADSVKG | 17 |
| | D05-VH | | | | |
| 71 | SRP1558-F01- | CDR-H2 | Kabat | VITDNSGNTDYADSVKG | 17 |
| | VH | | | | |
| 72 | SRP1448- | CDR-H2 | Kabat | VITGNSGTTDYADSVKG | 17 |
| | D09-VH | | | | |
| 73 | SRP1558- | CDR-H2 | Kabat | FITSDSGTTDYADSVKG | 17 |
| | A06-VH | | | | |
| 74 | SRP1558-E11- | CDR-H2 | Kabat | VISDDTGSTDYADSVKG | 17 |
| | VH | | | | |
| 75 | SRP1627- | CDR-H2 | Kabat | RIDPWNGDTEYAPKFQG | 17 |
| | A02-VH | | | | |
| 76 | SRP1627- | CDR-H2 | Kabat | RIDPWNGDTEYAPKFQG | 17 |
| | A11-VH | | | | |
| 77 | h5G11-2-VH | CDR-H2 | Kabat | WIDPENGDTEYAPKFQG | 17 |
| 78 | SRP1627- | CDR-H2 | Kabat | RIDPWNGDTEYAPKFQG | 17 |
| | B01-VH | | | | |
| 79 | 421.61.4. | CDR-H2 | Kabat | WIDPENGDTEYAPKFQG | 17 |
| | 5G11-VH | | | | |
| 80 | SRP1496- | CDR-H3 | | EIFG-FYWNPFDY | 12 |
| | A03-VH | | | | |
| 81 | SRP1496- | CDR-H3 | | EIFG-FYWNPFDY | 12 |
| | A04-VH | | | | |
| 82 | SRP1496- | CDR-H3 | | EIFG-FYWNPFDY | 12 |
| | B08-VH | | | | |
| 83 | SRP1648- | CDR-H3 | | EILG-FYWNPFDY | 12 |
| | B07-VH | | | | |
| 84 | SRP1648-E02- | CDR-H3 | | ESIG-FYLNPFDY | 12 |
| | VH | | | | |
| 85 | SRP1449- | CDR-H3 | | EWAVASWDYALDV | 13 |
| | B03-VH | | | | |
| 86 | SRP1449-F01- | CDR-H3 | | ESEVASWDYGLDV | 13 |
| | VH | | | | |
| 87 | SRP1449- | CDR-H3 | | EWAVSSWDYGMDV | 13 |
| | B07-VH | | | | |
| 88 | 1449-G09.2- | CDR-H3 | | EEAPENWDYALDV | 13 |
| | VH | | | | |
| 89 | SRP1449- | CDR-H3 | | EWADVSWDAGLDV | 13 |
| | D05-VH | | | | |
| 90 | SRP1558-F01- | CDR-H3 | | VFEGGVRPYS-DY | 12 |
| | VH | | | | |
| 91 | SRP1448- | CDR-H3 | | VYEGGVRPYS-DY | 12 |
| | D09-VH | | | | |
| 92 | SRP1558- | CDR-H3 | | VFEGGVRPFS-DY | 12 |
| | A06-VH | | | | |
| 93 | SRP1558-E11- | CDR-H3 | | VDNGGVRPYS-DY | 12 |
| | VH | | | | |
| 94 | SRP1627- | CDR-H3 | | SDALDY | 6 |
| | A02-VH | | | | |
| 95 | SRP1627- | CDR-H3 | | SDALDY | 6 |
| | A11-VH | | | | |
| 96 | h5G11-2-VH | CDR-H3 | | PDALDY | 6 |
| 97 | SRP1627- | CDR-H3 | | SEMVDY | 6 |
| | B01-VH | | | | |
| 98 | 421.61.4. | CDR-H3 | | PDALDY | 6 |
| | 5G11-VH | | | | |
| 99 | Linker | | | AAGSDQ | 6 |
| 100 | SRP1449- | CDR-L1 | | RASQ----SVSSSYLA | 12 |
| | D05-VL | | | | |
| 101 | SRP1449-F01- | CDR-L1 | | RASR----SVSSSYLA | 12 |
| | VL | | | | |
| 102 | 1449-G09.2- | CDR-L1 | | RASQ----SVSSSYLA | 12 |
| | VL | | | | |
| 103 | SRP1449- | CDR-L1 | | RASQ----SVSSSYLA | 12 |
| | B07-VL | | | | |
| 104 | SRP1449- | CDR-L1 | | RASQ----SVSSSYLA | 12 |
| | B03-VL | | | | |
| 105 | SRP1558-E11- | CDR-L1 | | RASQ----SVSSSYLA | 12 |
| | VL | | | | |
| 106 | SRP1558- | CDR-L1 | | RASQ----SVSSNPLA | 12 |
| | A06-VL | | | | |
| 107 | SRP1558-F01- | CDR-L1 | | RASQ----SVSSGNPA | 12 |
| | VL | | | | |
| 108 | SRP1448- | CDR-L1 | | RASQ----SVSSSYLA | 12 |
| | D09-VL | | | | |
| 109 | trastuzumab- | CDR-L1 | | RASQ----DVNTA-VA | 11 |
| | VL | | | | |
| 110 | SRP1627- | CDR-L1 | | KSSQSLLDSDGKTYLN | 16 |
| | A02-VL | | | | |
| 111 | SRP1627- | CDR-L1 | | KSSQSLLDSDGKTYLN | 16 |
| | A11-VL | | | | |
| 112 | SRP1627- | CDR-L1 | | KSSQSLLDSDGKTYLN | 16 |
| | B01-VL | | | | |
| 113 | h5G11-2-VL | CDR-L1 | | KSSQSLLDSDGKTYLN | 16 |
| 114 | 421.61.4. | CDR-L1 | | KSSQSLLDSDGKTYLN | 16 |
| | 5G11-VL | | | | |
| 115 | SRP1449- | CDR-L2 | | GASSRAT | 7 |
| | D05-VL | | | | |
| 116 | SRP1449-F01- | CDR-L2 | | GASSRAT | 7 |
| | VL | | | | |
| 117 | 1449-G09.2- | CDR-L2 | | GASSRAT | 7 |
| | VL | | | | |
| 118 | SRP1449- | CDR-L2 | | GASSRAT | 7 |
| | B07-VL | | | | |
| 119 | SRP1449- | CDR-L2 | | GASSRAT | 7 |
| | B03-VL | | | | |
| 120 | SRP1558-E11- | CDR-L2 | | GASSRAT | 7 |
| | VL | | | | |
| 121 | SRP1558- | CDR-L2 | | GASSRAT | 7 |
| | A06-VL | | | | |
| 122 | SRP1558-F01- | CDR-L2 | | GASSRAT | 7 |
| | VL | | | | |
| 123 | SRP1448- | CDR-L2 | | GASSRAT | 7 |
| | D09-VL | | | | |
| 124 | trastuzumab- | CDR-L2 | | SASFLYS | 7 |
| | VL | | | | |
| 125 | SRP1627- | CDR-L2 | | LVSKLDS | 7 |
| | A02-VL | | | | |
| 126 | SRP1627- | CDR-L2 | | LVSKLDS | 7 |
| | A11-VL | | | | |
| 127 | SRP1627- | CDR-L2 | | LVSKLDS | 7 |
| | B01-VL | | | | |
| 128 | h5G11-2-VL | CDR-L2 | | LVSKLDS | 7 |
| 129 | 421.61.4. | CDR-L2 | | LVSKLDS | 7 |
| | 5G11-VL | | | | |
| 130 | SRP1449- | CDR-L3 | | QQYGSTPFK | 9 |
| | D05-VL | | | | |
| 131 | SRP1449-F01- | CDR-L3 | | QQYGSSPFT | 9 |
| | VL | | | | |
| 132 | 1449-G09.2- | CDR-L3 | | QQYGRSPFS | 9 |
| | VL | | | | |
| 133 | SRP1449- | CDR-L3 | | QQYGASPFT | 9 |
| | B07-VL | | | | |
| 134 | SRP1449- | CDR-L3 | | QQYDRSPLT | 9 |
| | B03-VL | | | | |
| 135 | SRP1558-E11- | CDR-L3 | | QQYSLAPPT | 9 |
| | VL | | | | |
| 136 | SRP1558- | CDR-L3 | | QQYMAGPPT | 9 |
| | A06-VL | | | | |
| 137 | SRP1558-F01- | CDR-L3 | | QQYTAGPPT | 9 |
| | VL | | | | |
| 138 | SRP1448- | CDR-L3 | | QQDTAGPPT | 9 |
| | D09-VL | | | | |
| 139 | trastuzumab- | CDR-L3 | | QQHYTTPPT | 9 |
| | VL | | | | |
| 140 | SRP1627- | CDR-L3 | | SHGNPVPQT | 9 |
| | A02-VL | | | | |
| 141 | SRP1627- | CDR-L3 | | WHGINFPQT | 9 |
| | A11-VL | | | | |
| 142 | SRP1627- | CDR-L3 | | STYSHFPQT | 9 |
| | B01-VL | | | | |
| 143 | h5G11-2-VL | CDR-L3 | | WQGSHFPQT | 9 |
| 144 | 421.61.4. | CDR-L3 | | WQGSHFPQT | 9 |
| | 5G11-VL | | | | |
| 145 | scFvFc | scFv | | QVQLVESGGGVVQPGRSLRLSCAASG | 238 |
| | | | | FTFSSYGMHWVRQAPGKGLEWVAVIW | |
| | | | | YDGSYKYYADSVKGRFTISRDNSKNT | |
| | | | | LYLQMNSLRAEDTAVYYCAREEAPEN | |
| | | | | WDYALDVWGQGTTVTVSSGGGGSGGG | |
| | | | | GSGGGGSEIVLTQSPGTLSLSPGERA | |
| | | | | TLSCRASQSVSSSYLAWYQQKPGQKV | |
| | | | | DIK | |
| 146 | 421.61.4. | VH | | EVQLQQSGAELVRSGASVKLSCTASG | 115 |
| | 5G11-VH | | | FNIKDYYMHWVKQRPEQGLEWIAWID | |
| | | | | PENGDTEYAPKFQGRATLTADTSSNT | |
| | | | | AYLHLSSLTSEDTAVYYCNAPDALDY | |
| | | | | WGQGTSVTVSS | |
| 147 | SRP1627- | VH | | QVQLVQSGAEVKKPGSSVKVSCKASG | 115 |
| | A02-VH | | | FNINDYFMHWVRQAPGQGLEWIARID | |
| | | | | PWNGDTEYAPKFQGRVTITADESTST | |
| | | | | AYMELSSLRSEDTAVYYCGMSDALDY | |
| | | | | WGQGTLVTVSS | |
| 148 | SRP1627- | VH | | QVQLVQSGAEVKKPGSSVKVSCKASG | 115 |
| | A11-VH | | | FNINDYFMHWVRQAPGQGLEWIARID | |
| | | | | PWNGDTEYAPKFQGRVTITADESTST | |
| | | | | AYMELSSLRSEDTAVYYCGMSDALDY | |
| | | | | WGQGTLVTVSS | |
| 149 | SRP1627- | VH | | QVQLVQSGAEVKKPGSSVKVSCKASG | 115 |
| | B01-VH | | | FNITDLYMHWVRQAPGQGLEWIARID | |
| | | | | PWNGDTEYAPKFQGRATITADESTST | |
| | | | | AYMELSSLRSEDTAVYYCIASEMVDY | |
| | | | | WGQGTLVTVSS | |
| 150 | h5G11-2-VH | VH | | QVQLVQSGAEVKKPGSSVKVSCKASG | 115 |
| | | | | FNIKDYYMHWVRQAPGQGLEWIAWID | |
| | | | | PENGDTEYAPKFQGRVTITADESTST | |
| | | | | AYMELSSLRSEDTAVYYCNAPDALDY | |
| | | | | WGQGTLVTVSS | |
| 151 | SRP1449- | VH | | QVQLVESGGGVVQPGRSLRLSCAASG | 122 |
| | B03-VH | | | FTFSSYGMHWVRQAPGKGLEWVAAIW | |
| | | | | YDASYKYYADSVKGRFTISRDNSKNT | |
| | | | | LYLQMNSLRAEDTAVYYCAREWAVAS | |
| | | | | WDYALDVWGQGTTVTVSS | |
| 152 | SRP1449- | VH | | QVQLVESGGGVVQPGRSLRLSCAASG | 122 |
| | B07-VH | | | FTFSSYGMHWVRQAPGKGLEWVAVIW | |
| | | | | YDGSNKYYADSVKGRFTISRDNSKNT | |
| | | | | LYLQMNSLRAEDTAVYYCAREWAVSS | |
| | | | | WDYGMDVWGQGTTVTVSS | |
| 153 | SRP1449- | VH | | QVQLVESGGGVVQPGRSLRLSCAASG | 122 |
| | D05-VH | | | FTFRSFGMHWVRQAPGKGLEWVAVIW | |
| | | | | YDGSVKYYADSVKGRFTISRDNSKNT | |
| | | | | LYLQMNSLRAEDTAVYYCAREWADVS | |
| | | | | WDAGLDVWGQGTTVTVSS | |
| 154 | SRP1449-F01- | VH | | QVQLVESGGGVVQPGRSLRLSCAASG | 122 |
| | VH | | | FTFSSYGMHWVRQAPGKGLEWVAVIW | |
| | | | | YDGSYKYYADSVKGRFAISRDNSKNT | |
| | | | | LYLQMNSLRAEDTAVYYCARESEVAS | |
| | | | | WDYGLDVWGQGTTVTVSS | |
| 155 | 1449-G09.2- | VH | | QVQLVESGGGVVQPGRSLRLSCAASG | 122 |
| | VH | | | FTFSSYGMHWVRQAPGKGLEWVAVIW | |
| | | | | YDGSYKYYADSVKGRFTISRDNSKNT | |
| | | | | LYLQMNSLRAEDTAVYYCAREEAPEN | |
| | | | | WDYALDVWGQGTTVTVSS | |
| 156 | SRP1558- | VH | | EVQLLESGGGLVQPGGSLRLSCAASG | 121 |
| | A06-VH | | | FTFSESTMSWVRQAPGKGLEWVGFIT | |
| | | | | SDSGTTDYADSVKGRFTISRDNSKNT | |
| | | | | LYLQMNSLRAEDTAVYYCAKVFEGGV | |
| | | | | RPFSDYWGQGTLVTVSS | |
| 157 | SRP1558-E11- | VH | | EVQLLESGGGLVQPGGSLRLSCAASG | 121 |
| | VH | | | FTFTSSSMSWVRQAPGKGLEWVGVIS | |
| | | | | DDTGSTDYADSVKGRFTISRDNSKNT | |
| | | | | LYLQMNSLRAEDTAVYYCAKVDNGGV | |
| | | | | RPYSDYWGQGTLVTVSS | |
| 158 | SRP1558-F01- | VH | | EVQLLESGGGLVQPGGSLRLSCAASG | 121 |
| | VH | | | FTFPDSSMSWVRQAPGKGLEWVGVIT | |
| | | | | DNSGNTDYADSVKGRFTISRDNSKNT | |
| | | | | LYLQMNSLRAEDTAVYYCAKVFEGGV | |
| | | | | RPYSDYWGQGTLVTVSS | |
| 159 | SRP1448- | VH | | EVQLLESGGGLVQPGGSLRLSCAASG | 121 |
| | D09-VH | | | FTFTDSSMSWVRQAPGKGLEWVGVIT | |
| | | | | GNSGTTDYADSVKGRFTISRDNSKNT | |
| | | | | LYLQMNSLRAEDTAVYYCAKVYEGGV | |
| | | | | RPYSDYWGQGTLVTVSS | |
| 160 | SRP1496- | VH | | EVQLVESGGGLVQPGGSLRLSCAASG | 121 |
| | A03-VH | | | FNINDTYIHWVRQAPGKGLEWVGIID | |
| | | | | PYDGATDYADSVKGRFTISADTSKNT | |
| | | | | AYLQMNSLRAEDTAVYYCAREIFGFY | |
| | | | | WNPFDYWGQGTLVTVSS | |
| 161 | SRP1496- | VH | | EVQLVESGGGLVQPGGSLRLSCAASG | 121 |
| | A04-VH | | | FNINDTYIHWVRQAPGKGLEWVGIID | |
| | | | | PYDGATDYADSVKGRFTISADTSKNT | |
| | | | | AYLQMNSLRAEDTAVYYCAREIFGFY | |
| | | | | WNPFDYWGQGTLVTVSS | |
| 162 | SRP1496- | VH | | EVQLVESGGGLVQPGGSLRLSCAASG | 121 |
| | B08-VH | | | FNINDTYIHWVRQAPGKGLEWVGIID | |
| | | | | PYDGATDYADSVKGRFTISADTSKNT | |
| | | | | AYLQMNSLRAEDTAVYYCAREIFGFY | |
| | | | | WNPFDYWGQGTLVTVSS | |
| 163 | SRP1648- | VH | | EVQLVESGGGLVQPGGSLRLSCAASG | 121 |
| | B07-VH | | | FNIADTFIHWVRQAPGKGLEWVGIID | |
| | | | | PYDGDTDYADSVKGRFTISADTSKNT | |
| | | | | AYLQMNSLRAEDTAVYYCAREILGFY | |
| | | | | WNPFDYWGQGTLVTVSS | |
| 164 | SRP1648-E02- | VH | | EVQLVESGGGLVQPGGSLRLSCAASG | 121 |
| | VH | | | FNINDNYIHWVRQAPGKGLEWVGIID | |
| | | | | PYDGFTAYADSVKGRFTISADTSKNT | |
| | | | | AYLQMNSLRAEDTAVYYCARESIGFY | |
| | | | | LNPFDYWGQGTLVTVSS | |
| 165 | 421.61.4. | VL | | DVVMTQTPLTLSVTIGQIASISCKSS | 112 |
| | 5G11-VL | | | QSLLDSDGKTYLNWLLQRPGQSPKRL | |
| | | | | IYLVSKLDSGVPDRFTGSGSGTDFTL | |
| | | | | KISRVEAEDLGVYYCWQGSHFPQTFG | |
| | | | | GGTKLEIK | |
| 166 | SRP1627- | VL | | DVVMTQSPLSLPVTLGQPASISCKSS | 112 |
| | A02-VL | | | QSLLDSDGKTYLNWFQQRPGQSPRRL | |
| | | | | IYLVSKLDSGVPDRFSGSGSGTDFTL | |
| | | | | KISRVEAEDVGVYYCSHGNPVPQTFG | |
| | | | | QGTKVEIK | |
| 167 | SRP1627- | VL | | DVVMTQSPLSLPVTLGQPASISCKSS | 112 |
| | A11-VL | | | QSLLDSDGKTYLNWFQQRPGQSPRRL | |
| | | | | IYLVSKLDSGVPDRFSGSGSGTDFTL | |
| | | | | KISRVEAEDVGVYYCWHGINFPQTFG | |
| | | | | QGTKVEIK | |
| 168 | SRP1627- | VL | | DVVMTQSPLSLPVTLGQPASISCKSS | 112 |
| | B01-VL | | | QSLLDSDGKTYLNWFQQRPGQSPRRL | |
| | | | | IYLVSKLDSGVPDRFSGSGSGTDFTL | |
| | | | | KISRVEAEDVGVYYCSTYSHFPQTFG | |
| | | | | QGTKVEIK | |
| 169 | h5G11-2-VL | VL | | DVVMTQSPLSLPVTLGQPASISCKSS | 112 |
| | | | | QSLLDSDGKTYLNWFQQRPGQSPRRL | |
| | | | | IYLVSKLDSGVPDRFSGSGSGTDFTL | |
| | | | | KISRVEAEDVGVYYCWQGSHFPQTFG | |
| | | | | QGTKVEIK | |
| 170 | SRP1449- | VL | | EIVLTQSPGTMSLSPGERATLSCRAS | 108 |
| | B03-VL | | | QSVSSSYLAWYQQKPGQAPRLLIYGA | |
| | | | | SSRATGIPDRFSGSGSGTDFTLTISR | |
| | | | | LEPEDFAVYYCQQYDRSPLTFGPGTK | |
| | | | | VDIK | |
| 171 | SRP1449- | VL | | EIVLTQSPGTLSLSPGERATLSCRAS | 108 |
| | B07-VL | | | QSVSSSYLAWYQQKPGQAPRLLIYGA | |
| | | | | SSRATGIPNRFSGSGSGTDFTLTISR | |
| | | | | LEPEDFAVYYCQQYGASPFTFGPGTK | |
| | | | | VDIK | |
| 172 | SRP1449- | VL | | EIVLTQSPGTLSLSPGERATLSCRAS | 108 |
| | D05-VL | | | QSVSSSYLAWYQQKPGQAPRLLIYGA | |
| | | | | SSRATGIPDRFSGSGSGTDFTLTISR | |
| | | | | LEPEDFAVYYCQQYGSTPFKFGPGTK | |
| | | | | VDIK | |
| 173 | SRP1449-F01- | VL | | EIALTQSPGTLSLSPGERATLSCRAS | 108 |
| | VL | | | RSVSSSYLAWYQQKPGQAPRLLIYGA | |
| | | | | SSRATGIPDRFSGSGSGTDFTLTISR | |
| | | | | LEPEDFAVYYCQQYGSSPFTFGPGTK | |
| | | | | VDIK | |
| 174 | 1449-G09.2- | VL | | EIVLTQSPGTLSLSPGERATLSCRAS | 108 |
| | VL | | | QSVSSSYLAWYQQKPGQAPRLLIYGA | |
| | | | | SSRATGIPDRFSGSGSGTDFTLTISR | |
| | | | | LEPEDFAVYYCQQYGRSPFSFGPGTK | |
| | | | | VDIK | |
| 175 | SRP1558- | VL | | EIVLTQSPGTLSLSPGERATLSCRAS | 108 |
| | A06-VL | | | QSVSSNPLAWYQQKPGQAPRLLIYGA | |
| | | | | SSRATGIPDRFSGSGSGTDFTLTISR | |
| | | | | LEPEDFAVYYCQQYMAGPPTFGQGTK | |
| | | | | VEIK | |
| 176 | SRP1558-E11- | VL | | EIVLTQSPGTLSLSPGERATLSCRAS | 108 |
| | VL | | | QSVSSSYLAWYQQKPGQAPRLLIYGA | |
| | | | | SSRATGIPDRFSGSGSGTDFTLTISR | |
| | | | | LEPEDFAVYYCQQYSLAPPTLGQGTK | |
| | | | | VEIK | |
| 177 | SRP1558-F01- | VL | | EIVLTQSPGTLSLSPGERATLSCRAS | 108 |
| | VL | | | QSVSSGNPAWYQQKPGQAPRLLIYGA | |
| | | | | SSRATGIPDRFSGSGSGTDFTLTISR | |
| | | | | LEPXDFAVYYCQQYTAGPPTFGQGTK | |
| | | | | VEIK | |
| 178 | SRP1448- | VL | | EIVLTQSPGTLSLSPGERATLSCRAS | 108 |
| | D09-VL | | | QSVSSSYLAWYQQKPGQAPRLLIYGA | |
| | | | | SSRATGIPDRFSGSGSGTDFTLTISR | |
| | | | | LEPEDFAVYYCQQDTAGPPTFGQGTK | |
| | | | | VEIK | |
| 179 | trastuzumab- | VL | | DIQMTQSPSSLSASVGDRVTITCRAS | 107 |
| | VL | | | QDVNTAVAWYQQKPGKAPKLLIYSAS | |
| | | | | FLYSGVPSRFSGSRSGTDFTLTISSL | |
| | | | | QPEDFATYYCQQHYTTPPTFGQGTKV | |
| | | | | EIK | |
| 180 | IgG1 | | | ASTKGPSVFPLAPSSKSTSGGTAALG | 330 |
| | Constant | | | CLVKDYFPEPVTVSWNSGALTSGVHT | |
| | Region | | | FPAVLQSSGLYSLSSVVTVPSSSLGT | |
| | | | | QTYICNVNHKPSNTKVDKKVEPKSCD | |
| | | | | KTHTCPPCPAPELLGGPSVFLFPPKP | |
| | | | | KDTLMISRTPEVTCVVVDVSHEDPEV | |
| | | | | KFNWYVDGVEVHNAKTKPREEQYNST | |
| | | | | YRVVSVLTVLHQDWLNGKEYKCKVSN | |
| | | | | KALPAPIEKTISKAKGQPREPQVYTL | |
| | | | | PPSREEMTKNQVSLTCLVKGFYPSDI | |
| | | | | AVEWESNGQPENNYKTTPPVLDSDGS | |
| | | | | FFLYSKLTVDKSRWQQGNVFSCSVMH | |
| | | | | EALHNHYTQKSLSLSPGK | |
| 181 | Human IgG | LC | | RTVAAPSVFIFPPSDEQLKSGTASVV | 107 |
| | LC Ckappa | | | CLLNNFYPREAKVQWKVDNALQSGNS | |
| | | | | QESVTEQDSKDSTYSLSSTLTLSKAD | |
| | | | | YEKHKVYACEVTHQGLSSPVTKSFNR | |
| | | | | GEC | |
| 182 | Mouse IgG LC | LC | | RADAAPTVSIFPPSSEQLTSGGASVV | 107 |
| | Ckappa | | | CFLNNFYPKDINVKWKIDGSERQNGV | |
| | | | | LNSWTDQDSKDSTYSMSSTLTLTKDE | |
| | | | | YERHNSYTCEATHKTSTSPIVKSFNR | |
| | | | | NEC | |
| 183 | Human IgG1 | HC | | ASTKGPSVFPLAPSSKSTSGGTAALG | 330 |
| | HC | | | CLVKDYFPEPVTVSWNSGALTSGVHT | |
| | | | | FPAVLQSSGLYSLSSVVTVPSSSLGT | |
| | | | | QTYICNVNHKPSNTKVDKKVEPKSCD | |
| | | | | KTHTCPPCPAPELLGGPSVFLFPPKP | |
| | | | | KDTLMISRTPEVTCVVVDVSHEDPEV | |
| | | | | KFNWYVDGVEVHNAKTKPREEQYNST | |
| | | | | YRVVSVLTVLHQDWLNGKEYKCKVSN | |
| | | | | KALPAPIEKTISKAKGQPREPQVYTL | |
| | | | | PPSREEMTKNQVSLTCLVKGFYPSDI | |
| | | | | AVEWESNGQPENNYKTTPPVLDSDGS | |
| | | | | FFLYSKLTVDKSRWQQGNVFSCSVMH | |
| | | | | EALHNHYTQKSLSLSPGK | |
| 184 | Mouse IgG1 | HC | | AKTTPPSVYPLAPGSAAQTNSMVTLG | 323 |
| | HC | | | CLVKGYFPEPVTVTWNSGSLSSGVHT | |
| | | | | FPAVLQSDLYTLSSSVTVPSSTWPSE | |
| | | | | TVTCNVAHPASSTKVDKKIVPRDCGC | |
| | | | | KPCICTVPEVSSVFIFPPKPKDVLTI | |
| | | | | TLTPKVTCVVVDISKDDPEVQFSWFV | |
| | | | | DDVEVHTAQTQPREEQFNSTFRSVSE | |
| | | | | LPIMHQDWLNGKEFKCRVNSAAFPAP | |
| | | | | IEKTISKTKGRPKAPQVYTIPPPKEQ | |
| | | | | MAKDKVSLTCMITDFFPEDITVEWQW | |
| | | | | NGQPAENYKNTQPIMDTDGSYFVYSK | |
| | | | | LNVQKSNWEAGNTFTCSVLHEGLHNH | |
| | | | | HTEKSLSHSPG | |
| 185 | IgG1 Fc from | | | AAGSDQEPKSSDKTHTCPPCSAPELL | 252 |
| | scFv-Fc | | | GGSSVFLFPPKPKDTLMISRTPEVTC | |
| | | | | VVVDVSHEDPEVKFNWYVDGVEVHNA | |
| | | | | KTKPREEQYNSTYRVVSVLTVLHQDW | |
| | | | | LNGKEYKCKVSNKALPAPIEKTISKA | |
| | | | | KGQPREPQVYTLPPSRDELTKNQVSL | |
| | | | | TCLVKGFYPSDIAVEWESNGQPENNY | |
| | | | | KTTPPVLDSDGSFFLYSKLTVDKSRW | |
| | | | | QQGNVFSCSVMHEALHNHYTQKSLSL | |
| | | | | SPGK | |
| 186 | Lambda | | | GQPKAAPSVTLFPPSSEELQANKATL | 106 |
| | Constant | | | VCLISDFYPGAVTVAWKADSSPVKAG | |
| | Region | | | VETTTPSKQSNNKYAASSYLSLTPEQ | |
| | | | | WKSHRSYSCQVTHEGSTVEKTVAPTE | |
| | | | | CS | |
| 187 | Kappa | | | RTVAAPSVFIFPPSDEQLKSGTASVV | 107 |
| | Constant | | | CLLNNFYPREAKVQWKVDNALQSGNS | |
| | Region | | | QESVTEQDSKDSTYSLSSTLTLSKAD | |
| | | | | YEKHKVYACEVTHQGLSSPVTKSFNR | |
| | | | | GEC | |
| 188 | Linker | | | GGGGSGGGGSGGGGS | 15 |
| 189 | Linker | | | APGPSAPSHRSLPSRAFG | 18 |
| 190 | FLAG His Tag | | | GSGDYKDDDDKGSGHHHHHH | 20 |
| | with Linker | | | | |
| 191 | 26H10 | CDR-H1 | Chothia | GFTSSY | 6 |
| 192 | 26H10 | CDR-H1 | Kabat | SYGMH | 5 |
| 193 | 26H10 | CDR-H2 | Chothia | WYDGSN | 6 |
| 194 | 26H10 | CDR-H2 | Kabat | VIWYDGSNKYYADSVKG | 17 |
| 195 | 26H10 | CDR-H3 | | EWAVASWDYGMDV | 13 |
| 196 | 26H10 | CDR-L1 | | RASQ----SVSSSYLA | 12 |
| 197 | 26H10 | CDR-L2 | | GASSRAT | 7 |
| 198 | 26H10 | CDR-L3 | | QQYGSSPFT | 9 |
| 199 | 26H10 | VH | | QVQLVESGGGVVQPGRSLRLSCAASG | 122 |
| | | | | FTFSSYGMHWVRQAPGKGLEWVAVIW | |
| | | | | YDG | |
| | | | | SNKYYADSVKGRFTISRDNSKNTLYL | |
| | | | | QMNSLRAEDTAVYYCAREWAVASWDY | |
| | | | | GMDVWGQGTTVTVSS | |
| 200 | 26H10 | VL | | EIVLTQSPGTLSLSPGERATLSCRAS | 108 |
| | | | | QSVSSSYLAWYQQKPGQAPRLLIYGA | |
| | | | | SSRATGIPDRFSGSGSGTDFTLTISR | |
| | | | | LEPEDFAVYYCQQYGSSPFTFGPGTK | |
| | | | | VDIK | |
| 201 | 26H10 | scFV | | QVQLVESGGGVVQPGRSLRLSCAASG | 238 |
| | | | | FTFSSYGMHWVRQAPGKGLEWVAVIW | |
| | | | | YDGSNKYYADSVKGRFTISRDNSKNT | |
| | | | | LYLQMNSLRAEDTAVYYCAREWAVAS | |
| | | | | WDYGMDVWGQGTTVTVSSGGGGSGGG | |
| | | | | GSGGGGSEIVLTQSPGTLSLSPGERA | |
| | | | | TLSCRASQSVSSSYLAWYQQKPGQAP | |
| | | | | RLLIYGASSRATGIPDRFSGSGSGTD | |
| | | | | FTLTISRLEPEDFAVYYCQQYGSSPF | |
| | | | | TFGPGTKVDIK | |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

1. An isolated antibody that specifically binds to human LAG3, wherein the antibody comprises a CDR-H3 sequence selected from the group consisting of:
a. a sequence defined by the consensus sequence E-α₂-α₃-α₄-α₅-α₆-W-D-α₉-α₁₀-α₁₁-D-V where α₂ is S, W, or E; α₃ is A or E; α4 is V, P, or D; α₅ is A, S, E, or V; α6 is S or N, α₉ is Y or A; α₁₀ is A or G; and α₁₁ is L or M wherein when α₂ is W, α₄ is V, α₅ is A, α₆ is S, and α₁₀ is G, then α₁₁ is L, wherein when α₂ is W, α₄ is V, α₅ is A, α₆ is S, and α₁₀ is G, then α₁₁ is L; and
b. a sequence selected from SEQ ID Nos:80-98.

2. (canceled)

3. The antibody of claim 1, further comprising a CDR-L3 sequence selected from the group consisting of:
a. a sequence defined by the consensus sequence Q-Q-ι₃-ι₄-ι₅-ι₆-P-ι₈-ι₉, where ι₃ is Y or D; ι₄ is G, D, S, M, or T; ι₅ is R, S, A, or L; ι₆ is S, T, A, or G; ι₈ is F, L or P; and ι₉ is S, T, or K;
b. a sequence defined by the consensus sequence ι₁-ι₂-ι₃-ι₄-ι₅-ι₆-P-Q-T where ι₁ is S or W; ι₂ is H, T, or Q; ι₃ is G or Y; ι₄ is N, I, or S; and ι₅ is V or F; and
c. a sequence selected from SEQ ID NOs:130-144, or a variant thereof having three, two, or one amino acid substitution(s).

4. (canceled)

5. The antibody of claim 1, further comprising a Chothia CDR-H2 sequence selected from the group consisting of:
a. a sequence defined by the consensus sequence ε₁-ε₂-ε₃-ε₄-ε₅-ε₆, where ε₁ is D, W, or T; ε₂ is P, Y, D, G, or S; ε₃ is Y, D, N, W, or, E; ε₄ is D, A, G, S, T, or N; ε₅ is G or S; and ε₆ is A, D, F, Y, V, N, T, or S; and
b. a sequence selected from SEQ ID NOs:42-60, or a variant thereof having two or one amino acid substitutions(s).

6. (canceled)

7. The antibody of claim 1, further comprising a Chothia CDR-H1 sequence selected from the group consisting of:
a. a sequence defined by the consensus sequence G-F-γ₃-γ₄-γ₅-γ₆-γ₇, where γ₃ is N or T; γ₄ is I or F; γ₅ is K, N, A, S, R, P, or T; γ₆ is D, S, or E; and γ₇ is T, N, Y, F, S, or L;
b. a sequence defined by the consensus sequence G-F-T-F-δ₅-δ₆-δ₇, where δ₅ is S, R, P, T, or N; δ₆ is S, D, or E; and δ₇ is F, S, or Y; and
c. a sequence selected from SEQ ID NOs:4-22, or a variant thereof having two or one amino acid substitutions(s).

8. (canceled)

9. The antibody of claim 1, further comprising a Kabat CDR-H2 sequence selected from the group consisting of:
a. a sequence defined by the consensus sequence θ₁-I-θ₃-θ₄-θ₅-θ₆-θ₇-θ₈-θ₉-θ₁₀-Y-A-θ₁₃-θ₁₄-θ₁₅-θ₁₆-G, where θ₁ is I, A, V, R, or W; θ₃ is D, W, T, or S; θ₄ is P, Y, D, G, or S; θ₅ is Y, D, N, W, or E; θ₆ is D, A, G, S, T, or N; θ₇ is G or S; θ₈ is A, D, F, Y, N, V, T, or S; θ₉ is T or K; bio is D, A, Y or E; θ₁₃ is D, or P; θ₁₄ is S or K; θ₁₅ is V or F; and θ₁₆ is K; and
b. a sequence selected from SEQ ID NOs:61-79, or a variant thereof having three, two, or one amino acid substitutions(s).

10. (canceled)

11. The antibody of claim 1, further comprising a Kabat CDR-H1 sequence selected from the group consisting of:
a. a sequence defined by the consensus sequence ζ₁-ζ₂-ζ₃-ζ₄-ζ₅, where ζ₁ is D, S, or E; ζ₂ is T, N, Y, F, S, or L; ζ₃ is Y, F, G, S, or T; ζ₄ is I or M; and ζ₅ is H or S;
b. a sequence defined by the consensus sequence S-η₂-G-M-H, where η₂ is Y or F; and
c. a sequence selected from SEQ ID NOs:23-41, or a variant thereof having two or one amino acid substitutions.

12. (canceled)

13. The antibody of claim 1, further comprising a CDR-L2 sequence selected from: GASSRAT (SEQ ID NO:115), SASFLYS (SEQ ID NO:124), and LVSKLDS (SEQ ID NO:125), or a variant thereof having two or one amino acid substitution(s).

14. The antibody of claim 1, further comprising a CDR-L1 sequence selected from the group consisting of:
a. a sequence defined by the consensus R-A-S-Q-μ₅-μ₆-μ₇-μ₈-S-V-S-S-μ₁₃-μ₁₄-μ15-A, where μ₁₅ is absent; μ₆ is absent; μ₇ is absent; μ₈ is absent; μ₁₃ is S, N, or G; μ₁₄ is Y, P or N; and μ₁₅ is L or P; and
b. a sequence selected from SEQ ID NOs:100-114, or a variant thereof having three, two, or one amino acid substitution(s).

15. (canceled)

16. (canceled)

17. (canceled)

18. (canceled)

19. (canceled)

20. (canceled)

21. The antibody of claim 1, wherein the antibody comprises:
a. a V_{H} comprising: a CDR-H1 comprising one or more of SEQ ID Nos:12 and 31; a CDR-H2 comprising one or more of SEQ ID NOs:50 and 69; and a CDR-H3 comprising SEQ ID NO:88;
b. a V_{H} comprising: a CDR-H1 comprising one or more of SEQ ID NOs:4 and 23; a CDR-H2 comprising one or more of SEQ ID NOs:42 and 61; and a CDR-H3 comprising SEQ ID NO:80;
c. a V_{H} comprising: a CDR-H1 comprising one or more of SEQ ID NOs:5 and 24; a CDR-H2 comprising one or more of SEQ ID NOs:43 and 62; and a CDR-H3 comprising SEQ ID NO:81;
d. a V_{H} comprising: a CDR-H1 comprising one or more of SEQ ID NOs:6 and 25; a CDR-H2 comprising one or more of SEQ ID NOs:44 and 63; and a CDR-H3 comprising SEQ ID NO:82;
e. a V_{H} comprising: a CDR-H1 comprising one or more of SEQ ID NOs:7 and 26; a CDR-H2 comprising one or more of SEQ ID NOs:45 and 64; and a CDR-H3 comprising SEQ ID NO:83;
f. a V_{H} comprising: a CDR-H1 comprising one or more of SEQ ID NOs:8 and 27; a CDR-H2 comprising one or more of SEQ ID NOs:46 and 65; and a CDR-H3 comprising SEQ ID NO:84;
g. a V_{H} comprising: a CDR-H1 comprising one or more of SEQ ID NOs:9 and 28; a CDR-H2 comprising one or more of SEQ ID NOs:47 and 66; and a CDR-H3 comprising SEQ ID NO:85;
h. a V_{H} comprising: a CDR-H1 comprising one or more of SEQ ID NOs:10 and 29; a CDR-H2 comprising one or more of SEQ ID NOs:48 and 67; and a CDR-H3 comprising SEQ ID NO:86;
i. a V_{H} comprising: a CDR-H1 comprising one or more of SEQ ID NOs:11 and 30; a CDR-H2 comprising one or more of SEQ ID NOs:49 and 68; and a CDR-H3 comprising SEQ ID NO:87;
j. a V_{H} comprising: a CDR-H1 comprising one or more of SEQ ID NOs:13 and 32; a CDR-H2 comprising one or more of SEQ ID NOs:51 and 70; and a CDR-H3 comprising SEQ ID NO:89;
k. a V_{H} comprising: a CDR-H1 comprising one or more of SEQ ID NOs:14 and 33; a CDR-H2 comprising one or more of SEQ ID NOs:52 and 71; and a CDR-H3 comprising SEQ ID NO:90;
l. a V_{H} comprising: a CDR-H1 comprising one or more of SEQ ID NOs:15 and 34; a CDR-H2 comprising one or more of SEQ ID NOs:53 and 72; and a CDR-H3 comprising SEQ ID NO:91;
m. a V_{H} comprising: a CDR-H1 comprising one or more of SEQ ID NOs:16 and 35; a CDR-H2 comprising one or more of SEQ ID NOs:54 and 73; and a CDR-H3 comprising SEQ ID NO:92;
n. a V_{H} comprising: a CDR-H1 comprising one or more of SEQ ID NOs:17 and 36; a CDR-H2 comprising one or more of SEQ ID NOs:55 and 74; and a CDR-H3 comprising SEQ ID NO:93;
o. a V_{H} comprising: a CDR-H1 comprising one or more of SEQ ID NOs:18 and 37; a CDR-H2 comprising one or more of SEQ ID NOs:56 and 75; and a CDR-H3 comprising SEQ ID NO:94;
p. a V_{H} comprising: a CDR-H1 comprising one or more of SEQ ID NOs:19 and 38; a CDR-H2 comprising one or more of SEQ ID NOs:57 and 76; and a CDR-H3 comprising SEQ ID NO:95;
q. a V_{H} comprising: a CDR-H1 comprising one or more of SEQ ID NOs:20 and 39; a CDR-H2 comprising one or more of SEQ ID NOs:58 and 77; and a CDR-H3 comprising SEQ ID NO:96;
r. a V_{H} comprising: a CDR-H1 comprising one or more of SEQ ID NOs:21 and 40; a CDR-H2 comprising one or more of SEQ ID NOs:59 and 78; and a CDR-H3 comprising SEQ ID NO:97; and
s. a V_{H} comprising: a CDR-H1 comprising one or more of SEQ ID NOs:22 and 41; a CDR-H2 comprising one or more of SEQ ID NOs:60 and 79; and a CDR-H3 comprising SEQ ID NO:98.

22. The antibody of claim 21, wherein the V_{H} is selected from SEQ ID NOs:146-164.

23. The antibody of claim 21, wherein the antibody further comprises:
a. a V_{L} comprising: a CDR-L1 comprising SEQ ID NO:102; a CDR-L2 comprising SEQ ID NO:117; and a CDR-L3 comprising SEQ ID NO:132;
b. a V_{L} comprising: a CDR-L1 comprising SEQ ID NO:100; a CDR-L2 comprising SEQ ID NO:115; and a CDR-L3 comprising SEQ ID NO:130;
c. a V_{L} comprising: a CDR-L1 comprising SEQ ID NO:101; a CDR-L2 comprising SEQ ID NO:116; and a CDR-L3 comprising SEQ ID NO:131;
d. a V_{L} comprising: a CDR-L1 comprising SEQ ID NO:103; a CDR-L2 comprising SEQ ID NO:118; and a CDR-L3 comprising SEQ ID NO:133;
e. a V_{L} comprising: a CDR-L1 comprising SEQ ID NO:104; a CDR-L2 comprising SEQ ID NO:119; and a CDR-L3 comprising SEQ ID NO:134;
f. a V_{L} comprising: a CDR-L1 comprising SEQ ID NO:105; a CDR-L2 comprising SEQ ID NO:120; and a CDR-L3 comprising SEQ ID NO:135;
g. a V_{L} comprising: a CDR-L1 comprising SEQ ID NO:106; a CDR-L2 comprising SEQ ID NO:121; and a CDR-L3 comprising SEQ ID NO:136;
h. a V_{L} comprising: a CDR-L1 comprising SEQ ID NO:107; a CDR-L2 comprising SEQ ID NO:122; and a CDR-L3 comprising SEQ ID NO:137;
i. a V_{L} comprising: a CDR-L1 comprising SEQ ID NO:108; a CDR-L2 comprising SEQ ID NO:123; and a CDR-L3 comprising SEQ ID NO:138;
j. a V_{L} comprising: a CDR-L1 comprising SEQ ID NO:109; a CDR-L2 comprising SEQ ID NO:124; and a CDR-L3 comprising SEQ ID NO:139;
k. V_{L} comprising: a CDR-L1 comprising SEQ ID NO:110; a CDR-L2 comprising SEQ ID NO:125; and a CDR-L3 comprising SEQ ID NO:140;
l. a V_{L} comprising: a CDR-L1 comprising SEQ ID NO:111; a CDR-L2 comprising SEQ ID NO:126; and a CDR-L3 comprising SEQ ID NO:141;
m. a V_{L} comprising: a CDR-L1 comprising SEQ ID NO:112; a CDR-L2 comprising SEQ ID NO:127; and a CDR-L3 comprising SEQ ID NO:142;
n. V_{L} comprising: a CDR-L1 comprising SEQ ID NO:113; a CDR-L2 comprising SEQ ID NO:128; and a CDR-L3 comprising SEQ ID NO:143; and
o. a V_{L} comprising: a CDR-L1 comprising SEQ ID NO:114; a CDR-L2 comprising SEQ ID NO:129; and a CDR-L3 comprising SEQ ID NO:144.

24. The antibody of claim 23 wherein the V_{L} sequence is selected from SEQ ID NOs: 165-179.

25. (canceled)

26. (canceled)

27. (canceled)

28. The antibody of claim 24, wherein:
a. the V_{H} region is SEQ ID NO:155, or the variant thereof, and the V_{L} region is SEQ ID NO:174, or the variant thereof;
b. the V_{H} region is SEQ ID NO:146, or the variant thereof, and the V_{L} region is SEQ ID NO:165, or the variant thereof;
c. the V_{H} region is SEQ ID NO:147, or the variant thereof, and the V_{L} region is SEQ ID NO:166, or the variant thereof;
d. the V_{H} region is SEQ ID NO:148, or the variant thereof, and the V_{L} region is SEQ ID NO:167, or the variant thereof;
e. the V_{H} region is SEQ ID NO:149, or the variant thereof, and the V_{L} region is SEQ ID NO:168, or the variant thereof;
f. the V_{H} region is SEQ ID NO:150, or the variant thereof, and the V_{L} region is SEQ ID NO:169, or the variant thereof;
g. the V_{H} region is SEQ ID NO:151, or the variant thereof, and the V_{L} region is SEQ ID NO:170, or the variant thereof;
h. the V_{H} region is SEQ ID NO:152, or the variant thereof, and the V_{L} region is SEQ ID NO:171, or the variant thereof;
i. the V_{H} region is SEQ ID NO:153, or the variant thereof, and the V_{L} region is SEQ ID NO:172, or the variant thereof;
j. the V_{H} region is SEQ ID NO:154, or the variant thereof, and the V_{L} region is SEQ ID NO:173, or the variant thereof;
k. the V_{H} region is SEQ ID NO:156, or the variant thereof, and the V_{L} region is SEQ ID NO:175, or the variant thereof;
l. the V_{H} region is SEQ ID NO:157, or the variant thereof, and the V_{L} region is SEQ ID NO:176, or the variant thereof;
m. the V_{H} region is SEQ ID NO:158, or the variant thereof, and the V_{L} region is SEQ ID NO:177, or the variant thereof;
n. the V_{H} region is SEQ ID NO:159, or the variant thereof, and the V_{L} region is SEQ ID NO:178, or the variant thereof;
o. the V_{H} region is SEQ ID NO:160, or the variant thereof, and the V_{L} region is SEQ ID NO:179, or the variant thereof;
p. the V_{H} region is SEQ ID NO:161, or the variant thereof, and the V_{L} region is SEQ ID NO: 179, or the variant thereof;
q. the V_{H} region is SEQ ID NO:162, or the variant thereof, and the V_{L} region is SEQ ID NO: 179, or the variant thereof;
r. the V_{H} region is SEQ ID NO:164, or the variant thereof, and the V_{L} region is SEQ ID NO: 179, or the variant thereof; and
s. the V_{H} region is SEQ ID NO:163, or the variant thereof, and the V_{L} region is SEQ ID NO: 179, or the variant thereof.

29. (canceled)

30. (canceled)

31. The antibody of claim 1, wherein the isolated antibody inhibits binding of the second antibody to the LAG3 by at least 50%, or wherein the second antibody inhibits binding of the isolated antibody to the LAG3 by at least 50%.

32. The antibody of claim 1, wherein the antibody comprises at least one constant region domain.

33. The antibody of claim 32, wherein the constant region comprises a sequence selected from SEQ ID NOs:180-185.

34. The antibody of claim 1, wherein the antibody is a monoclonal antibody.

35. The antibody of claim 1, wherein the antibody is an IgA, an IgD, an IgE, an IgG, or an IgM.

36. The antibody of claim 1, wherein the antibody is humanized or human.

37. The antibody of claim 1, wherein the antibody is a glycosylated.

38. The antibody of claim 1, wherein the antibody is an antibody fragment.

39. The antibody of claim 38, wherein the antibody fragment is selected from an Fv fragment, a Fab fragment, a F(ab′)₂ fragment, a Fab′ fragment, an scFv (sFv) fragment, and an scFv-Fc fragment.

40. The antibody of claim 39, wherein the antibody is an scFv fragment.

41. The antibody of claim 40, wherein the scFv fragment comprises SEQ ID NO: 145, with or without the N-terminal M residue.

42. The antibody of claim 39, wherein the antibody is an scFv-Fc fragment.

43. The antibody of claim 42, wherein the scFv-Fc fragment comprises SEQ ID NO: 145, with or without the N-terminal M residue, and SEQ ID NO:185.

44. The antibody of claim 1, wherein the antibody has a kₐ of about 5.02×10⁴ M⁻¹×sec⁻¹ to about 5.31×10⁷M⁻¹×sec⁻¹ when associating with human LAG3 at a temperature of 25° C.

45. The antibody of claim 1, wherein the antibody has a k_{d} of about 2.79×10⁻² sec⁻¹ to about 6.78×10⁻⁵ sec⁻¹ when dissociating from human LAG3 at a temperature of 25° C.

46. The antibody of claim 1, wherein the antibody has a K_{D} of about 1.3×10⁻⁸ M to about 1.93×10⁻¹⁰ M when bound to human LAG3 at a temperature of 25° C.

47. The antibody of claim 1, wherein the antibody specifically binds cynomolgus LAG3.

48. The antibody of claim 47, wherein the antibody has a K_{D} of 1.6×10⁻⁹ M to about 0.3×10⁻⁹ M when bound to cynomolgus LAG3 at a temperature of 25° C.

49. The antibody of claim 48, wherein the ratio of K_{D} for human LAG3 to K_{D} for cynomolgus LAG3 is about 0.25 to about 4.0.

50. A kit comprising the antibody of claim 21, and instructions for use of the antibody.

51. (canceled)

52. (canceled)

53. A polynucleotide encoding the antibody of claim 1.

54. A vector comprising the polynucleotide of claim 53.

55. A recombinant host cell comprising the vector of claim 54.

56. (canceled)

57. (canceled)

58. A cell-free expression reaction comprising the vector of claim 54.

59. A pharmaceutical composition comprising the antibody of claim 1 and a pharmaceutically acceptable carrier.

60. A method of treating or preventing a disease or condition in a subject in need thereof, comprising administering to the subject an effective amount of an antibody of claim 1.

61. (canceled)

62. The method of claim 60, wherein the disease or condition is a cancer.

63. An isolated antibody that specifically binds to human LAG3, wherein the antibody comprises:
a. a CDR-H3 sequence selected from the group consisting of:
i. a sequence defined by the consensus sequence E-α₂-α₃-α₄-α₅-α₆-W-D-α₉-α₁₀-α₁₁-D-V where α₂ is S, W, or E; α₃ is A or E; α₄ is V, P, or D; α₅ is A, S, E, or V; α₆ is S or N; α₉ is Y or A; α₁₀ is A or G; and α₁₁ is L or M wherein when α₂ is W, α₄ is V, α₅ is A, α₆ is S, and α₁₀ is G, then α₁₁ is L, wherein when α₂ is W, α₄ is V, α₅ is A, α₆ is S, and α₁₀ is G, then α₁₁ is L; and
ii. a sequence selected from SEQ ID Nos:80-98;
b. a CDR-H2 sequence selected from the group consisting of:
i. a sequence defined by the consensus sequence ε₁-ε₂-ε₃-ε₄-ε₅-ε₆, where ε₁ is D, W, or T; ε₂ is P, Y, D, G, or S; ε₃ is Y, D, N, W, or, E; ε₄ is D, A, G, S, T, or N; ε₅ is G or S; and ε₆ is A, D, F, Y, V, N, T, or S;
ii. a sequence defined by the consensus sequence θ₁-I-θ₃-θ₄-θ₅-θ₆-θ₇-θ₈-θ₉-θ₁₀-Y-A-θ₁₃-θ₁₄-θ₁₅-θ₁₆-G, where θ₁ is I, A, V, R, or W; θ₃ is D, W, T, or S; θ₄ is P, Y, D, G, or S; θ₅ is Y, D, N, W, or E; θ₆ is D, A, G, S, T, or N; θ₇ is G or S; θ₈ is A, D, F, Y, N, V, T, or S; θ₉ is T or K; θ₁₀ is D, A, Y or E; θ₁₃ is D, or P; θ₁₄ is S or K; θ₁₅ is V or F; and θ₁₆ is K;
iii. a sequence selected from SEQ ID NOs:42-60, or a variant thereof having two or one amino acid substitutions(s); and
iv. a sequence selected from SEQ ID NOs:61-79, or a variant thereof having three, two, or one amino acid substitutions(s);
c. a CDR-H1 sequence selected from the group consisting of:
i. a sequence defined by the consensus sequence G-F-T-F-δ₅-δ₆-δ₇, where δ₅ is S, R, P, T, or N; δ₆ is S, D, or E; and δ₇ is F, S, or Y;
ii. a sequence defined by the consensus sequence S-η₂-G-M-H, where η₂ is Y or F;
iii. a sequence selected from SEQ ID NOs:4-22, or a variant thereof having two or one amino acid substitutions(s); and
iv. a sequence selected from SEQ ID NOs:23-41, or a variant thereof having two or one amino acid substitutions;
d. a CDR-L3 sequence selected from the group consisting of:
i. a sequence defined by the consensus sequence Q-Q-ι₃-ι₄-ι₅-ι₆-P-ι₈-ι₉, where ι₃ is Y or D; ι₄ is G, D, S, M, or T; ι₅ is R, S, A, or L; ι₆ is S, T, A, or G; ι₈ is F, L or P; and ι₉ is S, T, or K; and
ii. a sequence selected from SEQ ID NOs:130-144, or a variant thereof having two or one amino acid substitution(s);
e. a CDR-L2 sequence selected from the group consisting of: GASSRAT (SEQ ID NO:115), SASFLYS (SEQ ID NO:124), and LVSKLDS (SEQ ID NO:125), or a variant thereof having two or one amino acid substitution(s); and
f. a CDR-L1 sequence selected from the group consisting of:
i. a sequence defined by the consensus R-A-S-Q-μ₅-μ₆-μ₇-μ₈-S-V-S-S-μ₁₃-μ₁₄-μ₁₅-A, where μ5 is absent; μ₆ is absent; μ₇ is absent; μ₈ is absent; μ₁₃ is S, N, or G; μ₁₄ is Y, P or N; and μ₁₅ is L or P; and
ii. a sequence selected from SEQ ID NOs:100-114, or a variant thereof having three, two, or one amino acid substitution(s).


SEQUENCE LISTING (201 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 533 aa, Macaca sp.
  misc_feature (74)..(74): Xaa can be any naturally occurring amino acid
MWEAQFLGLLFLQPLWVAPVKPPQPGAEISVVWAQEGAPAQLPCSPTIPLQDLSLLRRAGVTWQHQPDSGPPAXAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHLPLFLILGVLFLLLLVTGAFGFHLWRRQWRPRRFSALEQGIHPPQAQSKIEELEQEPELEPEPELERELGPEPEPGPEPEPEQL

SEQ ID NO: 3 - protein, 521 aa, Mus musculus
MREDLLLGFLLLGLLWEAPVVSSGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRISGDLKGGHLVLVLILGALSLFLLVAGAFGFHWWRKQLLLRRFSALEHGIQPFPAQRKIEELERELETEMGQEPEPEPEPQLEPEPRQL

SEQ ID NO: 4 - protein, 7 aa, Artificial Sequence
  Synthetic SRP1496-A03-VH
GFNINDT

SEQ ID NO: 5 - protein, 7 aa, Artificial Sequence
  Synthetic SRP1496-A04-VH
GFNINDT

SEQ ID NO: 6 - protein, 7 aa, Artificial Sequence
  Synthetic SRP1496-B08-VH
GFNINDT

SEQ ID NO: 7 - protein, 7 aa, Artificial Sequence
  Synthetic SRP1648-B07-VH
GFNIADT

SEQ ID NO: 8 - protein, 7 aa, Artificial Sequence
  Synthetic SRP1648-E02-VH
GFNINDN

SEQ ID NO: 9 - protein, 7 aa, Artificial Sequence
  Synthetic SRP1449-B03-VH
GFTFSSY

SEQ ID NO: 10 - protein, 7 aa, Artificial Sequence
  Synthetic SRP1449-F01-VH
GFTFSSY

SEQ ID NO: 11 - protein, 7 aa, Artificial Sequence
  Synthetic SRP1449-B07-VH
GFTFSSY

SEQ ID NO: 12 - protein, 7 aa, Artificial Sequence
  Synthetic 1449-G09.2-VH
GFTFSSY

SEQ ID NO: 13 - protein, 7 aa, Artificial Sequence
  Synthetic SRP1449-D05-VH
GFTFRSF

SEQ ID NO: 14 - protein, 7 aa, Artificial Sequence
  Synthetic SRP1558-F01-VH
GFTFPDS

SEQ ID NO: 15 - protein, 7 aa, Artificial Sequence
  Synthetic SRP1448-D09-VH
GFTFTDS

SEQ ID NO: 16 - protein, 7 aa, Artificial Sequence
  Synthetic SRP1558-A06-VH
GFTFSES

SEQ ID NO: 17 - protein, 7 aa, Artificial Sequence
  Synthetic SRP1558-E11-VH
GFTFTSS

SEQ ID NO: 18 - protein, 7 aa, Artificial Sequence
  Synthetic SRP1627-A02-VH
GFNINDY

SEQ ID NO: 19 - protein, 7 aa, Artificial Sequence
  Synthetic SRP1627-A11-VH
GFNINDY

SEQ ID NO: 20 - protein, 7 aa, Artificial Sequence
  Synthetic h5G11-2-VH
GFNIKDY

SEQ ID NO: 21 - protein, 7 aa, Artificial Sequence
  Synthetic SRP1627-B01-VH
GFNITDL

SEQ ID NO: 22 - protein, 7 aa, Artificial Sequence
  Synthetic 421.61.4.5G11-VH
GFNIKDY

SEQ ID NO: 23 - protein, 5 aa, Artificial Sequence
  Synthetic SRP1496-A03-VH
DTYIH

SEQ ID NO: 24 - protein, 5 aa, Artificial Sequence
  Synthetic SRP1496-A04-VH
DTYIH

SEQ ID NO: 25 - protein, 5 aa, Artificial Sequence
  Synthetic SRP1496-B08-VH
DTYIH

SEQ ID NO: 26 - protein, 5 aa, Artificial Sequence
  Synthetic SRP1648-B07-VH
DTFIH

SEQ ID NO: 27 - protein, 5 aa, Artificial Sequence
  Synthetic SRP1648-E02-VH
DNYIH

SEQ ID NO: 28 - protein, 5 aa, Artificial Sequence
  Synthetic SRP1449-B03-VH
SYGMH

SEQ ID NO: 29 - protein, 5 aa, Artificial Sequence
  Synthetic SRP1449-F01-VH
SYGMH

SEQ ID NO: 30 - protein, 5 aa, Artificial Sequence
  Synthetic SRP1449-B07-VH
SYGMH

SEQ ID NO: 31 - protein, 5 aa, Artificial Sequence
  Synthetic 1449-G09.2-VH
SYGMH

SEQ ID NO: 32 - protein, 5 aa, Artificial Sequence
  Synthetic SRP1449-D05-VH
SFGMH

SEQ ID NO: 33 - protein, 5 aa, Artificial Sequence
  Synthetic SRP1558-F01-VH
DSSMS

SEQ ID NO: 34 - protein, 5 aa, Artificial Sequence
  Synthetic SRP1448-D09-VH
DSSMS

SEQ ID NO: 35 - protein, 5 aa, Artificial Sequence
  Synthetic SRP1558-A06-VH
ESTMS

SEQ ID NO: 36 - protein, 5 aa, Artificial Sequence
  Synthetic SRP1558-E11-VH
SSSMS

SEQ ID NO: 37 - protein, 5 aa, Artificial Sequence
  Synthetic SRP1627-A02-VH
DYFMH

SEQ ID NO: 38 - protein, 5 aa, Artificial Sequence
  Synthetic SRP1627-A11-VH
DYFMH

SEQ ID NO: 39 - protein, 5 aa, Artificial Sequence
  Synthetic h5G11-2-VH
DYYMH

SEQ ID NO: 40 - protein, 5 aa, Artificial Sequence
  Synthetic SRP1627-B01-VH
DLYMH

SEQ ID NO: 41 - protein, 5 aa, Artificial Sequence
  Synthetic 421.61.4.5G11-VH
DYYMH

SEQ ID NO: 42 - protein, 6 aa, Artificial Sequence
  Synthetic SRP1496-A03-VH
DPYDGA

SEQ ID NO: 43 - protein, 6 aa, Artificial Sequence
  Synthetic SRP1496-A04-VH
DPYDGA

SEQ ID NO: 44 - protein, 6 aa, Artificial Sequence
  Synthetic SRP1496-B08-VH
DPYDGA

SEQ ID NO: 45 - protein, 6 aa, Artificial Sequence
  Synthetic SRP1648-B07-VH
DPYDGD

SEQ ID NO: 46 - protein, 6 aa, Artificial Sequence
  Synthetic SRP1648-E02-VH
DPYDGF

SEQ ID NO: 47 - protein, 6 aa, Artificial Sequence
  Synthetic SRP1449-B03-VH
WYDASY

SEQ ID NO: 48 - protein, 6 aa, Artificial Sequence
  Synthetic SRP1449-F01-VH
WYDGSY

SEQ ID NO: 49 - protein, 6 aa, Artificial Sequence
  Synthetic SRP1449-B07-VH
WYDGSN

SEQ ID NO: 50 - protein, 6 aa, Artificial Sequence
  Synthetic 1449-G09.2-VH
WYDGSY

SEQ ID NO: 51 - protein, 6 aa, Artificial Sequence
  Synthetic SRP1449-D05-VH
WYDGSV

SEQ ID NO: 52 - protein, 6 aa, Artificial Sequence
  Synthetic SRP1558-F01-VH
TDNSGN

SEQ ID NO: 53 - protein, 6 aa, Artificial Sequence
  Synthetic SRP1448-D09-VH
TGNSGT

SEQ ID NO: 54 - protein, 6 aa, Artificial Sequence
  Synthetic SRP1558-A06-VH
TSDSGT

SEQ ID NO: 55 - protein, 6 aa, Artificial Sequence
  Synthetic SRP1558-E11-VH
SDDTGS

SEQ ID NO: 56 - protein, 6 aa, Artificial Sequence
  Synthetic SRP1627-A02-VH
DPWNGD

SEQ ID NO: 57 - protein, 6 aa, Artificial Sequence
  Synthetic SRP1627-A11-VH
DPWNGD

SEQ ID NO: 58 - protein, 6 aa, Artificial Sequence
  Synthetic h5G11-2-VH
DPENGD

SEQ ID NO: 59 - protein, 6 aa, Artificial Sequence
  Synthetic SRP1627-B01-VH
DPWNGD

SEQ ID NO: 60 - protein, 6 aa, Artificial Sequence
  Synthetic 421.61.4.5G11-VH
DPENGD

SEQ ID NO: 61 - protein, 17 aa, Artificial Sequence
  Synthetic SRP1496-A03-VH
IIDPYDGATDYADSVKG

SEQ ID NO: 62 - protein, 17 aa, Artificial Sequence
  Synthetic SRP1496-A04-VH
IIDPYDGATDYADSVKG

SEQ ID NO: 63 - protein, 17 aa, Artificial Sequence
  Synthetic SRP1496-B08-VH
IIDPYDGATDYADSVKG

SEQ ID NO: 64 - protein, 17 aa, Artificial Sequence
  Synthetic SRP1648-B07-VH
IIDPYDGDTDYADSVKG

SEQ ID NO: 65 - protein, 17 aa, Artificial Sequence
  Synthetic SRP1648-E02-VH
IIDPYDGFTAYADSVKG

SEQ ID NO: 66 - protein, 17 aa, Artificial Sequence
  Synthetic SRP1449-B03-VH
AIWYDASYKYYADSVKG

SEQ ID NO: 67 - protein, 17 aa, Artificial Sequence
  Synthetic SRP1449-F01-VH
VIWYDGSYKYYADSVKG

SEQ ID NO: 68 - protein, 17 aa, Artificial Sequence
  Synthetic SRP1449-B07-VH
VIWYDGSNKYYADSVKG

SEQ ID NO: 69 - protein, 17 aa, Artificial Sequence
  Synthetic 1449-G09.2-VH
VIWYDGSYKYYADSVKG

SEQ ID NO: 70 - protein, 17 aa, Artificial Sequence
  Synthetic SRP1449-D05-VH
VIWYDGSVKYYADSVKG

SEQ ID NO: 71 - protein, 17 aa, Artificial Sequence
  Synthetic SRP1558-F01-VH
VITDNSGNTDYADSVKG

SEQ ID NO: 72 - protein, 17 aa, Artificial Sequence
  Synthetic SRP1448-D09-VH
VITGNSGTTDYADSVKG

SEQ ID NO: 73 - protein, 17 aa, Artificial Sequence
  Synthetic SRP1558-A06-VH
FITSDSGTTDYADSVKG

SEQ ID NO: 74 - protein, 17 aa, Artificial Sequence
  Synthetic SRP1558-E11-VH
VISDDTGSTDYADSVKG

SEQ ID NO: 75 - protein, 17 aa, Artificial Sequence
  Synthetic SRP1627-A02-VH
RIDPWNGDTEYAPKFQG

SEQ ID NO: 76 - protein, 17 aa, Artificial Sequence
  Synthetic SRP1627-A11-VH
RIDPWNGDTEYAPKFQG

SEQ ID NO: 77 - protein, 17 aa, Artificial Sequence
  Synthetic h5G11-2-VH
WIDPENGDTEYAPKFQG

SEQ ID NO: 78 - protein, 17 aa, Artificial Sequence
  Synthetic SRP1627-B01-VH
RIDPWNGDTEYAPKFQG

SEQ ID NO: 79 - protein, 17 aa, Artificial Sequence
  Synthetic 421.61.4.5G11-VH
WIDPENGDTEYAPKFQG

SEQ ID NO: 80 - protein, 12 aa, Artificial Sequence
  Synthetic SRP1496-A03-VH
EIFGFYWNPFDY

SEQ ID NO: 81 - protein, 12 aa, Artificial Sequence
  Synthetic SRP1496-A04-VH
EIFGFYWNPFDY

SEQ ID NO: 82 - protein, 12 aa, Artificial Sequence
  Synthetic SRP1496-B08-VH
EIFGFYWNPFDY

SEQ ID NO: 83 - protein, 12 aa, Artificial Sequence
  Synthetic SRP1648-B07-VH
EILGFYWNPFDY

SEQ ID NO: 84 - protein, 12 aa, Artificial Sequence
  Synthetic SRP1648-E02-VH
ESIGFYLNPFDY

SEQ ID NO: 85 - protein, 13 aa, Artificial Sequence
  Synthetic SRP1449-B03-VH
EWAVASWDYALDV

SEQ ID NO: 86 - protein, 13 aa, Artificial Sequence
  Synthetic SRP1449-F01-VH
ESEVASWDYGLDV

SEQ ID NO: 87 - protein, 13 aa, Artificial Sequence
  Synthetic SRP1449-B07-VH
EWAVSSWDYGMDV

SEQ ID NO: 88 - protein, 13 aa, Artificial Sequence
  Synthetic 1449-G09.2-VH
EEAPENWDYALDV

SEQ ID NO: 89 - protein, 13 aa, Artificial Sequence
  Synthetic SRP1449-D05-VH
EWADVSWDAGLDV

SEQ ID NO: 90 - protein, 12 aa, Artificial Sequence
  Synthetic SRP1558-F01-VH
VFEGGVRPYSDY

SEQ ID NO: 91 - protein, 12 aa, Artificial Sequence
  Synthetic SRP1448-D09-VH
VYEGGVRPYSDY

SEQ ID NO: 92 - protein, 12 aa, Artificial Sequence
  Synthetic SRP1558-A06-VH
VFEGGVRPFSDY

SEQ ID NO: 93 - protein, 12 aa, Artificial Sequence
  Synthetic SRP1558-E11-VH
VDNGGVRPYSDY

SEQ ID NO: 94 - protein, 6 aa, Artificial Sequence
  Synthetic SRP1627-A02-VH
SDALDY

SEQ ID NO: 95 - protein, 6 aa, Artificial Sequence
  Synthetic SRP1627-A11-VH
SDALDY

SEQ ID NO: 96 - protein, 6 aa, Artificial Sequence
  Synthetic h5G11-2-VH
PDALDY

SEQ ID NO: 97 - protein, 6 aa, Artificial Sequence
  Synthetic SRP1627-B01-VH
SEMVDY

SEQ ID NO: 98 - protein, 6 aa, Artificial Sequence
  Synthetic 421.61.4.5G11-VH
PDALDY

SEQ ID NO: 99 - protein, 6 aa, Artificial Sequence
  Synthetic Linker
AAGSDQ

SEQ ID NO: 100 - protein, 12 aa, Artificial Sequence
  Synthetic SRP1449-D05-VL
RASQSVSSSYLA

SEQ ID NO: 101 - protein, 12 aa, Artificial Sequence
  Synthetic SRP1449-F01-VL
RASRSVSSSYLA

SEQ ID NO: 102 - protein, 12 aa, Artificial Sequence
  Synthetic 1449-G09.2-VL
RASQSVSSSYLA

SEQ ID NO: 103 - protein, 12 aa, Artificial Sequence
  Synthetic SRP1449-B07-VL
RASQSVSSSYLA

SEQ ID NO: 104 - protein, 12 aa, Artificial Sequence
  Synthetic SRP1449-B03-VL
RASQSVSSSYLA

SEQ ID NO: 105 - protein, 12 aa, Artificial Sequence
  Synthetic SRP1558-E11-VL
RASQSVSSSYLA

SEQ ID NO: 106 - protein, 12 aa, Artificial Sequence
  Synthetic SRP1558-A06-VL
RASQSVSSNPLA

SEQ ID NO: 107 - protein, 12 aa, Artificial Sequence
  Synthetic SRP1558-F01-VL
RASQSVSSGNPA

SEQ ID NO: 108 - protein, 12 aa, Artificial Sequence
  Synthetic SRP1448-D09-VL
RASQSVSSSYLA

SEQ ID NO: 109 - protein, 11 aa, Artificial Sequence
  Synthetic trastuzumab-VL
RASQDVNTAVA

SEQ ID NO: 110 - protein, 16 aa, Artificial Sequence
  Synthetic SRP1627-A02-VL
KSSQSLLDSDGKTYLN

SEQ ID NO: 111 - protein, 16 aa, Artificial Sequence
  Synthetic SRP1627-A11-VL
KSSQSLLDSDGKTYLN

SEQ ID NO: 112 - protein, 16 aa, Artificial Sequence
  Synthetic SRP1627-B01-VL
KSSQSLLDSDGKTYLN

SEQ ID NO: 113 - protein, 16 aa, Artificial Sequence
  Synthetic h5G11-2-VL
KSSQSLLDSDGKTYLN

SEQ ID NO: 114 - protein, 16 aa, Artificial Sequence
  Synthetic 421.61.4.5G11-VL
KSSQSLLDSDGKTYLN

SEQ ID NO: 115 - protein, 7 aa, Artificial Sequence
  Synthetic SRP1449-D05-VL
GASSRAT

SEQ ID NO: 116 - protein, 7 aa, Artificial Sequence
  Synthetic SRP1449-F01-VL
GASSRAT

SEQ ID NO: 117 - protein, 7 aa, Artificial Sequence
  Synthetic 1449-G09.2-VL
GASSRAT

SEQ ID NO: 118 - protein, 7 aa, Artificial Sequence
  Synthetic SRP1449-B07-VL
GASSRAT

SEQ ID NO: 119 - protein, 7 aa, Artificial Sequence
  Synthetic SRP1449-B03-VL
GASSRAT

SEQ ID NO: 120 - protein, 7 aa, Artificial Sequence
  Synthetic SRP1558-E11-VL
GASSRAT

SEQ ID NO: 121 - protein, 7 aa, Artificial Sequence
  Synthetic SRP1558-A06-VL
GASSRAT

SEQ ID NO: 122 - protein, 7 aa, Artificial Sequence
  Synthetic SRP1558-F01-VL
GASSRAT

SEQ ID NO: 123 - protein, 7 aa, Artificial Sequence
  Synthetic SRP1448-D09-VL
GASSRAT

SEQ ID NO: 124 - protein, 7 aa, Artificial Sequence
  Synthetic trastuzumab-VL
SASFLYS

SEQ ID NO: 125 - protein, 7 aa, Artificial Sequence
  Synthetic SRP1627-A02-VL
LVSKLDS

SEQ ID NO: 126 - protein, 7 aa, Artificial Sequence
  Synthetic SRP1627-A11-VL
LVSKLDS

SEQ ID NO: 127 - protein, 7 aa, Artificial Sequence
  Synthetic SRP1627-B01-VL
LVSKLDS

SEQ ID NO: 128 - protein, 7 aa, Artificial Sequence
  Synthetic h5G11-2-VL
LVSKLDS

SEQ ID NO: 129 - protein, 7 aa, Artificial Sequence
  Synthetic 421.61.4.5G11-VL
LVSKLDS

SEQ ID NO: 130 - protein, 9 aa, Artificial Sequence
  Synthetic SRP1449-D05-VL
QQYGSTPFK

SEQ ID NO: 131 - protein, 9 aa, Artificial Sequence
  Synthetic SRP1449-F01-VL
QQYGSSPFT

SEQ ID NO: 132 - protein, 9 aa, Artificial Sequence
  Synthetic 1449-G09.2-VL
QQYGRSPFS

SEQ ID NO: 133 - protein, 9 aa, Artificial Sequence
  Synthetic SRP1449-B07-VL
QQYGASPFT

SEQ ID NO: 134 - protein, 9 aa, Artificial Sequence
  Synthetic SRP1449-B03-VL
QQYDRSPLT

SEQ ID NO: 135 - protein, 9 aa, Artificial Sequence
  Synthetic SRP1558-E11-VL
QQYSLAPPT

SEQ ID NO: 136 - protein, 9 aa, Artificial Sequence
  Synthetic SRP1558-A06-VL
QQYMAGPPT

SEQ ID NO: 137 - protein, 9 aa, Artificial Sequence
  Synthetic SRP1558-F01-VL
QQYTAGPPT

SEQ ID NO: 138 - protein, 9 aa, Artificial Sequence
  Synthetic SRP1448-D09-VL
QQDTAGPPT

SEQ ID NO: 139 - protein, 9 aa, Artificial Sequence
  Synthetic trastuzumab-VL
QQHYTTPPT

SEQ ID NO: 140 - protein, 9 aa, Artificial Sequence
  Synthetic SRP1627-A02-VL
SHGNPVPQT

SEQ ID NO: 141 - protein, 9 aa, Artificial Sequence
  Synthetic SRP1627-A11-VL
WHGINFPQT

SEQ ID NO: 142 - protein, 9 aa, Artificial Sequence
  Synthetic SRP1627-B01-VL
STYSHFPQT

SEQ ID NO: 143 - protein, 9 aa, Artificial Sequence
  Synthetic h5G11-2-VL
WQGSHFPQT

SEQ ID NO: 144 - protein, 9 aa, Artificial Sequence
  Synthetic 421.61.4.5G11-VL
WQGSHFPQT

SEQ ID NO: 145 - protein, 185 aa, Artificial Sequence
  Synthetic scFvFc
QVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSGGGGSGGGGSGGGGSEIVLTQSPGTLSLSPGERATLSCRASQSVSSSYLAWYQQKPGQKVDIK

SEQ ID NO: 146 - protein, 115 aa, Artificial Sequence
  Synthetic 421.61.4.5G11-VH
EVQLQQSGAELVRSGASVKLSCTASGFNIKDYYMHWVKQRPEQGLEWIAWIDPENGDTEYAPKFQGRATLTADTSSNTAYLHLSSLTSEDTAVYYCNAPDALDYWGQGTSVTVSS

SEQ ID NO: 147 - protein, 115 aa, Artificial Sequence
  Synthetic SRP1627-A02-VH
QVQLVQSGAEVKKPGSSVKVSCKASGFNINDYFMHWVRQAPGQGLEWIARIDPWNGDTEYAPKFQGRVTITADESTSTAYMELSSLRSEDTAVYYCGMSDALDYWGQGTLVTVSS

SEQ ID NO: 148 - protein, 115 aa, Artificial Sequence; identical to SEQ ID NO: 147
  Synthetic SRP1627-A11-VH

SEQ ID NO: 149 - protein, 115 aa, Artificial Sequence
  Synthetic SRP1627-B01-VH
QVQLVQSGAEVKKPGSSVKVSCKASGFNITDLYMHWVRQAPGQGLEWIARIDPWNGDTEYAPKFQGRATITADESTSTAYMELSSLRSEDTAVYYCIASEMVDYWGQGTLVTVSS

SEQ ID NO: 150 - protein, 115 aa, Artificial Sequence
  Synthetic h5G11-2-VH
QVQLVQSGAEVKKPGSSVKVSCKASGFNIKDYYMHWVRQAPGQGLEWIAWIDPENGDTEYAPKFQGRVTITADESTSTAYMELSSLRSEDTAVYYCNAPDALDYWGQGTLVTVSS

SEQ ID NO: 151 - protein, 122 aa, Artificial Sequence
  Synthetic SRP1449-B03-VH
QVQLVESGGGVVQPGRSLRLSCAASGFTFSSYGMHWVRQAPGKGLEWVAAIWYDASYKYYADSVKGRFTISRDNSKNTLYLQMNSLRAEDTAVYYCAREWAVASWDYALDVWGQGTTVTVSS

SEQ ID NO: 152 - protein, 122 aa, Artificial Sequence
  Synthetic SRP1449-B07-VH
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVSSWDYGMDVWGQGTTVTVSS

SEQ ID NO: 153 - protein, 122 aa, Artificial Sequence
  Synthetic SRP1449-D05-VH
QVQLVESGGGVVQPGRSLRLSCAASGFTFRSFGMHWVRQAPGKGLEWVAVIWYDGSVKYYADSVKGRFTISRDNSKNTLYLQMNSLRAEDTAVYYCAREWADVSWDAGLDVWGQGTTVTVSS

SEQ ID NO: 154 - protein, 122 aa, Artificial Sequence
  Synthetic SRP1449-F01-VH
QVQLVESGGGVVQPGRSLRLSCAASGFTFSSYGMHWVRQAPGKGLEWVAVIWYDGSYKYYADSVKGRFAISRDNSKNTLYLQMNSLRAEDTAVYYCARESEVASWDYGLDVWGQGTTVTVSS

SEQ ID NO: 155 - protein, 122 aa, Artificial Sequence
  Synthetic 1449-G09.2-VH
QVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSS

SEQ ID NO: 156 - protein, 121 aa, Artificial Sequence
  Synthetic SRP1558-A06-VH
EVQLLESGGGLVQPGGSLRLSCAASGFTFSESTMSWVRQAPGKGLEWVGFITSDSGTTDYADSVKGRFTISRDNSKNTLYLQMNSLRAEDTAVYYCAKVFEGGVRPFSDYWGQGTLVTVSS

SEQ ID NO: 157 - protein, 121 aa, Artificial Sequence
  Synthetic SRP1558-E11-VH
EVQLLESGGGLVQPGGSLRLSCAASGFTFTSSSMSWVRQAPGKGLEWVGVISDDTGSTDYADSVKGRFTISRDNSKNTLYLQMNSLRAEDTAVYYCAKVDNGGVRPYSDYWGQGTLVTVSS

SEQ ID NO: 158 - protein, 121 aa, Artificial Sequence
  Synthetic SRP1558-F01-VH
EVQLLESGGGLVQPGGSLRLSCAASGFTFPDSSMSWVRQAPGKGLEWVGVITDNSGNTDYADSVKGRFTISRDNSKNTLYLQMNSLRAEDTAVYYCAKVFEGGVRPYSDYWGQGTLVTVSS

SEQ ID NO: 159 - protein, 121 aa, Artificial Sequence
  Synthetic SRP1448-D09-VH
EVQLLESGGGLVQPGGSLRLSCAASGFTFTDSSMSWVRQAPGKGLEWVGVITGNSGTTDYADSVKGRFTISRDNSKNTLYLQMNSLRAEDTAVYYCAKVYEGGVRPYSDYWGQGTLVTVSS

SEQ ID NO: 160 - protein, 121 aa, Artificial Sequence
  Synthetic SRP1496-A03-VH
EVQLVESGGGLVQPGGSLRLSCAASGFNINDTYIHWVRQAPGKGLEWVGIIDPYDGATDYADSVKGRFTISADTSKNTAYLQMNSLRAEDTAVYYCAREIFGFYWNPFDYWGQGTLVTVSS

SEQ ID NO: 161 - protein, 121 aa, Artificial Sequence; identical to SEQ ID NO: 160
  Synthetic SRP1496-A04-VH

SEQ ID NO: 162 - protein, 121 aa, Artificial Sequence; identical to SEQ ID NO: 160
  Synthetic SRP1496-B08-VH

SEQ ID NO: 163 - protein, 121 aa, Artificial Sequence
  Synthetic SRP1648-B07-VH
EVQLVESGGGLVQPGGSLRLSCAASGFNIADTFIHWVRQAPGKGLEWVGIIDPYDGDTDYADSVKGRFTISADTSKNTAYLQMNSLRAEDTAVYYCAREILGFYWNPFDYWGQGTLVTVSS

SEQ ID NO: 164 - protein, 121 aa, Artificial Sequence
  Synthetic SRP1648-E02-VH
EVQLVESGGGLVQPGGSLRLSCAASGFNINDNYIHWVRQAPGKGLEWVGIIDPYDGFTAYADSVKGRFTISADTSKNTAYLQMNSLRAEDTAVYYCARESIGFYLNPFDYWGQGTLVTVSS

SEQ ID NO: 165 - protein, 112 aa, Artificial Sequence
  Synthetic 421.61.4.5G11-VL
DVVMTQTPLTLSVTIGQIASISCKSSQSLLDSDGKTYLNWLLQRPGQSPKRLIYLVSKLDSGVPDRFTGSGSGTDFTLKISRVEAEDLGVYYCWQGSHFPQTFGGGTKLEIK

SEQ ID NO: 166 - protein, 112 aa, Artificial Sequence
  Synthetic SRP1627-A02-VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCSHGNPVPQTFGQGTKVEIK

SEQ ID NO: 167 - protein, 112 aa, Artificial Sequence
  Synthetic SRP1627-A11-VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWHGINFPQTFGQGTKVEIK

SEQ ID NO: 168 - protein, 112 aa, Artificial Sequence
  Synthetic SRP1627-B01-VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCSTYSHFPQTFGQGTKVEIK

SEQ ID NO: 169 - protein, 112 aa, Artificial Sequence
  Synthetic h5G11-2-VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGSHFPQTFGQGTKVEIK

SEQ ID NO: 170 - protein, 108 aa, Artificial Sequence
  Synthetic SRP1449-B03-VL
EIVLTQSPGTMSLSPGERATLSCRASQSVSSSYLAWYQQKPGQAPRLLIYGASSRATGIPDRFSGSGSGTDFTLTISRLEPEDFAVYYCQQYDRSPLTFGPGTKVDIK

SEQ ID NO: 171 - protein, 108 aa, Artificial Sequence
  Synthetic SRP1449-B07-VL
EIVLTQSPGTLSLSPGERATLSCRASQSVSSSYLAWYQQKPGQAPRLLIYGASSRATGIPNRFSGSGSGTDFTLTISRLEPEDFAVYYCQQYGASPFTFGPGTKVDIK

SEQ ID NO: 172 - protein, 108 aa, Artificial Sequence
  Synthetic SRP1449-D05-VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSTPFKFGPGTKVDIK

SEQ ID NO: 173 - protein, 108 aa, Artificial Sequence
  Synthetic SRP1449-F01-VL
EIALTQSPGTLSLSPGERATLSCRASRSVSSSYLAWYQQKPGQAPRLLIYGASSRATGIPDRFSGSGSGTDFTLTISRLEPEDFAVYYCQQYGSSPFTFGPGTKVDIK

SEQ ID NO: 174 - protein, 108 aa, Artificial Sequence
  Synthetic 1449-G09.2-VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIK

SEQ ID NO: 175 - protein, 108 aa, Artificial Sequence
  Synthetic SRP1558-A06-VL
EIVLTQSPGTLSLSPGERATLSCRASQSVSSNPLAWYQQKPGQAPRLLIYGASSRATGIPDRFSGSGSGTDFTLTISRLEPEDFAVYYCQQYMAGPPTFGQGTKVEIK

SEQ ID NO: 176 - protein, 108 aa, Artificial Sequence
  Synthetic SRP1558-E11-VL
EIVLTQSPGTLSLSPGERATLSCRASQSVSSSYLAWYQQKPGQAPRLLIYGASSRATGIPDRFSGSGSGTDFTLTISRLEPEDFAVYYCQQYSLAPPTLGQGTKVEIK

SEQ ID NO: 177 - protein, 108 aa, Artificial Sequence
  Synthetic SRP1558-F01-VL
  misc_feature (82)..(82): Xaa can be any naturally occurring amino acid
EIVLTQSPGTLSLSPGERATLSCRASQSVSSGNPAWYQQKPGQAPRLLIYGASSRATGIPDRFSGSGSGTDFTLTISRLEPXDFAVYYCQQYTAGPPTFGQGTKVEIK

SEQ ID NO: 178 - protein, 108 aa, Artificial Sequence
  Synthetic SRP1448-D09-VL
EIVLTQSPGTLSLSPGERATLSCRASQSVSSSYLAWYQQKPGQAPRLLIYGASSRATGIPDRFSGSGSGTDFTLTISRLEPEDFAVYYCQQDTAGPPTFGQGTKVEIK

SEQ ID NO: 179 - protein, 107 aa, Artificial Sequence [sequence omitted]

SEQ ID NO: 180 - protein, 330 aa, Artificial Sequence
  Synthetic IgG1 Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 107 aa, Homo sapiens [sequence omitted]

SEQ ID NO: 182 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 183 - protein, 330 aa, Homo sapiens; identical to SEQ ID NO: 180

SEQ ID NO: 184 - protein, 323 aa, Mus musculus
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 185 - protein, 238 aa, Artificial Sequence
  Synthetic IgG1 Fc from scFv-Fc
AAGSDQEPKSSDKTHTCPPCSAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 106 aa, Artificial Sequence
  Synthetic Lambda Constant Region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 187 - protein, 107 aa, Artificial Sequence [sequence omitted]

SEQ ID NO: 188 - protein, 15 aa, Artificial Sequence
  Synthetic Linker
GGGGSGGGGSGGGGS

SEQ ID NO: 189 - protein, 18 aa, Artificial Sequence
  Synthetic Linker
APGPSAPSHRSLPSRAFG

SEQ ID NO: 190 - protein, 20 aa, Artificial Sequence
  Synthetic FLAG His Tag with Linker
GSGDYKDDDDKGSGHHHHHH

SEQ ID NO: 191 - protein, 6 aa, Artificial Sequence
  Synthetic 26H10
GFTSSY

SEQ ID NO: 192 - protein, 5 aa, Artificial Sequence
  Synthetic 26H10
SYGMH

SEQ ID NO: 193 - protein, 6 aa, Artificial Sequence
  Synthetic 26H10
WYDGSN

SEQ ID NO: 194 - protein, 17 aa, Artificial Sequence
  Synthetic 26H10
VIWYDGSNKYYADSVKG

SEQ ID NO: 195 - protein, 13 aa, Artificial Sequence
  Synthetic 26H10
EWAVASWDYGMDV

SEQ ID NO: 196 - protein, 12 aa, Artificial Sequence
  Synthetic 26H10
RASQSVSSSYLA

SEQ ID NO: 197 - protein, 7 aa, Artificial Sequence
  Synthetic 26H10
GASSRAT

SEQ ID NO: 198 - protein, 9 aa, Artificial Sequence
  Synthetic 26H10
QQYGSSPFT

SEQ ID NO: 199 - protein, 122 aa, Artificial Sequence
  Synthetic 26H10
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSS

SEQ ID NO: 200 - protein, 108 aa, Artificial Sequence
  Synthetic 26H10
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 201 - protein, 245 aa, Artificial Sequence
  Synthetic 26H10
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK